(19) United States
(12) Patent Application Publication
(10) Pub. No.: US 2018/0353435 A1
(43) Pub. Date: Dec. 13, 2018

(54) POLYMER-LIPID MATERIALS FOR DELIVERY OF NUCLEIC ACIDS

(71) Applicant: Massachusetts Institute of Technology, Cambridge, MA (US)

(72) Inventors: Michael Mitchell, Cambridge, MA (US); Amanda Chung, Malden, MA (US); Pedro P. G. Guimaraes, Revere, MA (US); Omar F. Khan, Cambridge, MA (US); Robert S. Langer, Newton, MA (US); Daniel Griffith Anderson, Framingham, MA (US)

(21) Appl. No.: 16/002,965

(22) Filed: Jun. 7, 2018

Related U.S. Application Data
(60) Provisional application No. 62/516,574, filed on Jun. 7, 2017.

(51) Int. Cl. A61K 9/51; C12N 15/113

(52) U.S. Cl. CPC A61K 9/5146; A61K 9/5123; C12N 2320/32; C12N 2310/14; C12N 15/1138

(57) ABSTRACT

The present invention provides compositions (e.g., nanoparticles) comprising a conjugated polyethyleneimine (PEI) polymer (a “conjugated lipomer”), or a pharmaceutically acceptable salt thereof, and a lipid-PEG conjugate, wherein the conjugated lipomer of Formula (I) contains one or more groups of the formula (iii). Compositions of the invention are useful for the delivery of active agents, for example, for the treatment of disease.

### RELATED APPLICATIONS

This application claims priority under 35 U.S.C. § 119(e) to U.S. Provisional Applications No. 62/516,574, filed Jun. 7, 2017, the content of which is incorporated herein by reference in its entirety.

### GOVERNMENT FUNDING

This invention was made with Government support under Contract No. W81XWH-14-1-0100 awarded by the U.S. Army Medical Research and Material Command, and under Grant No. R01 EB016101 awarded by the National Institutes of Health. The Government has certain rights in the invention.

### BACKGROUND OF THE INVENTION

Human bone marrow harbors about 10,000 bona fide hematopoietic stem cells as well as millions of downstream progenitors and releases billions of blood cells into the circulation every day (1). The organ produces a cellular ensemble that accomplishes vital tasks including oxygen transport, defense against pathogens and clotting (1, 2). The activities of its inhabitants, such as cell quiescence, proliferation, differentiation and migration, are adjusted to current systemic needs and regulated by non-hematopoietic bone marrow niche cells (2, 3). This cast of supporting cells includes endothelial cells, which instruct hematopoietic cell behavior via a mix of soluble and cell surface-bound signals (1, 4). Niche cells receive circulating and neuronal signals from outside the marrow and relay them to hematopoietic stem and progenitor cells (HSPC) (5).

Over the past decade, many niche cell steady-state functions have been discovered, leading to approved drugs for stem cell mobilization prior to transplantation (6). Drugs, such as Filgrastim, that disrupt the interactions between SDF-1 and its receptor CXCR4 on leukocytes and HSPC are now widely utilized as agents to mobilize stem cells into the bloodstream for bone marrow transplantation (7). Such agents have primarily been applied in the realm of hematology/oncology; however, recent evidence suggests that leukocyte and HSPC release from bone marrow plays an essential role in many other chronic inflammatory conditions, including cardiovascular disease (8). Broadly speaking, the number of circulating leukocytes and the production of blood components in the hematopoietic niche correlate closely with mortality, and if the bone marrow fails altogether, the organism succumbs within a week or two (9, 10). Therefore, technologies that modulate cell behavior within the hematopoietic niche could improve our fundamental understanding and treatment of a range of disease processes that are governed by bone marrow-derived leukocytes.

RNA interference (RNAi) therapeutics are a potentially attractive means to influence protein expression within the hematopoietic niche, as they can be used to silence nearly any gene within the body to achieve therapeutic effects (11). Because the gene sequences are known, siRNA drugs can be screened for in silico, produced and validated within very short time spans. However, while potent siRNAs can be rapidly identified, systemic delivery to the appropriate tissue can prove challenging.

Nucleic acids (e.g., siRNA) cannot easily be delivered effectively to bone marrow cells in vivo. Thus, in vivo therapeutics for the treatment of bone marrow diseases are limited to small molecules, antibodies, and proteins. Currently the only methods to deliver nucleic acids to bone marrow cells are ex vivo, where cells are harvested from patients and then subsequently treated with nucleic acids. These cells are then administered systemically to patients, where the engraftment of the treated cells back into bone marrow is extremely low (<1%). New technology useful in treating and targeting bone marrow cells with nucleic acids directly within patients, for treating a range of diseases including bone marrow cancers, immunological disorders, and hematological disorders, is therefore needed.

One such technology is the use of nanoparticles as drug delivery vehicles (13). For siRNA delivery, nanoparticle's key advantages are: (i) preventing nucleic acid degradation by serum endonucleases in blood, (ii) avoiding renal clearance from the bloodstream, (iii) delivering cargo to specific cells by tailoring nanoparticle surface chemistry and (iv) mediating target cell entry and endosomal escape to enable nucleic acid release into the cytoplasm (11, 14). Delivery materials differ in efficiency, toxicity and biodistribution, and certain nanoparticles have avidity to certain cell types, tissues and organs (15), particularly to hepatocytes, leukocytes and endothelial cells (16-22). It has previously been reported that nanoparticulate formulation, consisting of low molecular weight polyamines and lipids, that mediated potent gene silencing in endothelial cells residing in the lung (17). Nanoparticulate formulations that mediate potent gene silencing in the hematopoietic niche have not been previously reported.

### SUMMARY OF THE INVENTION

The present disclosure describes the development of an siRNA formulation capable of delivering siRNA to endothelial cells in the hematopoietic niche. A library of nanoparticles based on a new class of nanoparticle-forming materials generated by combinatorial chemical synthesis was first screened. The materials were synthesized by reacting low-molecular weight polyamines with epoxide-terminated lipids using an epoxide ring-opening reaction. By screening a library of these nanoparticles in vivo, a new polymer-lipid hybrid nanoparticle capable of efficient delivery to bone marrow endothelial cells was developed. In a series of proof-of-concept experiments, endothelial cell expression of two quintessential hematopoietic niche factors was silenced, thereby altering HSPC behavior and systemic leukocyte supply.

The present invention provides a composition (e.g., nanoparticle) comprising both: a conjugated polyethyleneimine (PEI) polymer (also referred to herein as a “conjugated lipomer” or “lipomer”) of Formula (I); and a lipid-polyethylene glycol (PEG) conjugate of Formula (II). In certain embodiments, the particle is a nanoparticle. Compositions (e.g., pharmaceutical compositions) of the particle are also provided. In certain embodiments, the particle, or composition, is useful, for example, as a delivery system for biologically active agents (e.g., nucleic acids to bone marrow in vivo). In certain embodiments, the particle, or composition, is useful for targeting bone marrow. The conjugated polyethyleneimine polymers are preferably prepared from low molecular weight linear polyethyleneimine (LPEI) and branched polyethyleneimine (BPEI) polymers, i.e., having a number average molar mass (Mn) of ≤2000 (i.e., approximately ≤2 kDa).

In one aspect of the invention, provided is a composition comprising a conjugated lipomer of Formula (I):

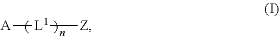

or a pharmaceutically acceptable salt thereof,
wherein:

each instance of L¹ is independently selected from the formulae:

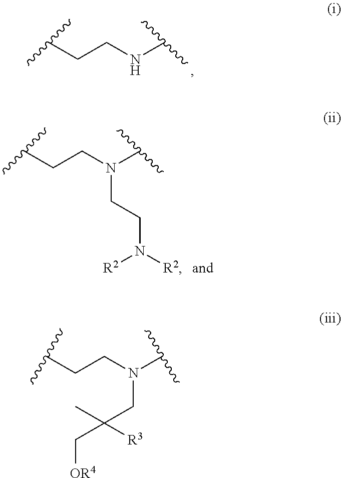

provided that at least one L¹ is selected from formula (iii);

n is an integer between 3 to 45, inclusive;

each instance of R² is independently hydrogen; acyl; silyl; sulfonyl; an amino protecting group; substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; substituted or unsubstituted carbocyclyl; substituted or unsubstituted heterocyclyl; substituted or unsubstituted aryl; substituted or unsubstituted heteroaryl; a substituted or unsubstituted polyethyleneimine; or a group of the formula (iii′):

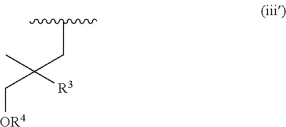

or the two R² groups are joined to form a substituted or unsubstituted heterocyclyl;

each instance of R³ is independently substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; substituted or unsubstituted carbocyclyl; substituted or unsubstituted heterocyclyl; substituted or unsubstituted aryl; substituted or unsubstituted heteroaryl; or a hydrophilic polymer;

each instance of R⁴ is independently hydrogen, acyl; silyl; a hydroxyl protecting group; substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; substituted or unsubstituted carbocyclyl; substituted or unsubstituted heterocyclyl; substituted or unsubstituted aryl; or substituted or unsubstituted heteroaryl;

A is —N(R⁵)₂, wherein each instance of R⁵ is independently hydrogen; acyl; silyl; sulfonyl; an amino protecting group; substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; substituted or unsubstituted carbocyclyl; substituted or unsubstituted heterocyclyl; substituted or unsubstituted aryl; substituted or unsubstituted heteroaryl; or a group of the formula (iii′):

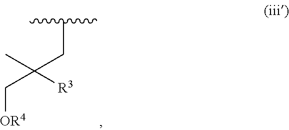

or two R⁵ groups are joined to form a substituted or unsubstituted heterocyclyl; and

Z is hydrogen; acyl; silyl; sulfonyl; an amino protecting group; substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; substituted or unsubstituted carbocyclyl; substituted or unsubstituted heterocyclyl; substituted or unsubstituted aryl; substituted or unsubstituted heteroaryl, or a group of the formula (iii′):

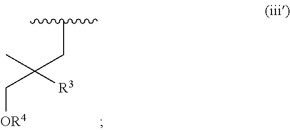

or Z and the nitrogen atom to which it is attached form a substituted or unsubstituted heterocyclyl group; and
a lipid-polyethylene glycol (PEG) conjugate of Formula (II):

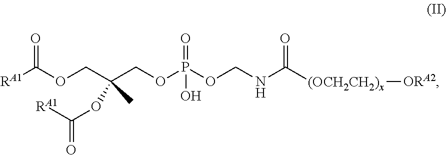

or a pharmaceutically acceptable salt thereof,
wherein:

each instance of R^{A1} is independently unsubstituted C₆-C₂₀ alkyl;

R^{A2} is substituted or unsubstituted alkyl; and

x is an integer between 15 to 135, inclusive.

In certain embodiments, the particle is a nanoparticle.

Provided are compositions comprising the conjugated lipomers in the form of a particle (e.g., a nanoparticle or microparticle). For example, in certain embodiments, provided is a composition including a particle, wherein the particle comprises a conjugated lipomer, e.g., a conjugated lipomer of Formula (I), and, optionally an excipient. In certain embodiments, the composition is a pharmaceutical composition or a cosmetic composition. In certain embodiments, the particle encapsulates an agent, e.g., an agent to be delivered. In certain embodiments, the biologically active agent is delivered to the subject in vivo. In certain embodiments, the agent is delivered to bone marrow in vivo. In certain embodiments, the biologically active agent is delivered to bone marrow cells of the subject. In certain embodiments, the composition further comprises an agent. In certain embodiments, the composition further comprises a biologically active agent (e.g., a therapeutic or diagnostic agent). In certain embodiments, the agent is a small molecule, organometallic compound, nucleic acid, protein, peptide, polynucleotide, metal, targeting agent, an isotopically labeled chemical compound, drug, vaccine, immunological agent. In certain embodiments, the agent is a polynucleotide (e.g., DNA or RNA). In certain embodiments, the RNA is RNAi, dsRNA, siRNA, shRNA, miRNA, or antisense RNA. In certain embodiments, the polynucleotide and the one or more conjugated lipomers are not covalently attached.

In certain embodiments, the conjugated lipomer is of the formula:

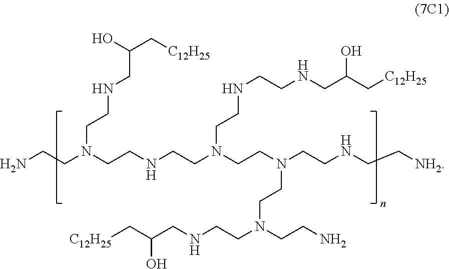

In certain embodiments, the lipid-PEG conjugate of Formula (II) is of the formula:

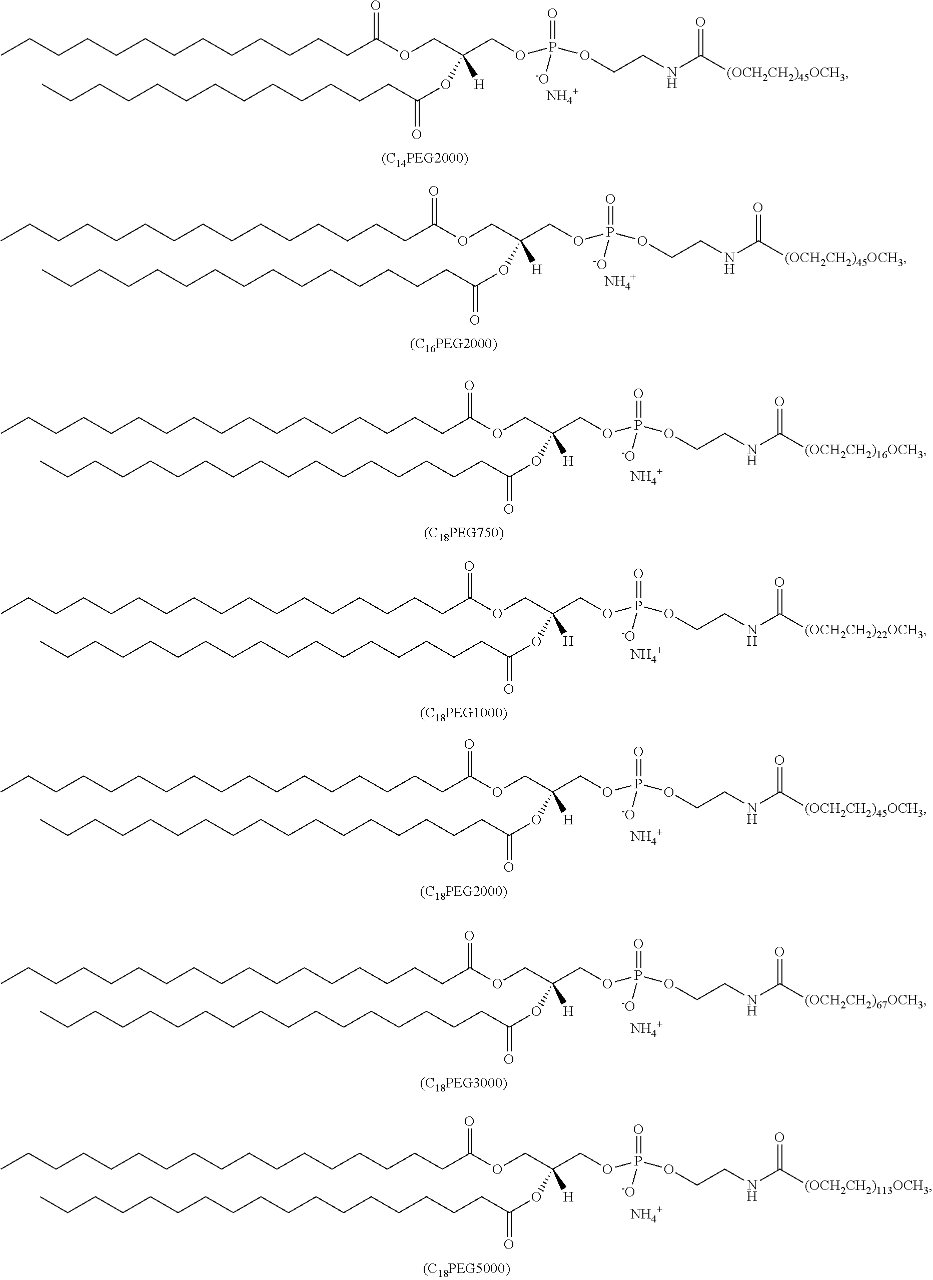

or a pharmaceutically acceptable salt thereof.
In certain embodiments, the composition includes a particle, wherein the particle comprises a conjugated lipomer of Formula (I); and a lipid-polyethylene glycol (PEG) conjugate of Formula (II). In certain embodiments, the particle is a nanoparticle. In certain embodiments, the composition comprises about 90 molar percent of conjugated lipomer (e.g., 7C1) and about 10 molar percent of C₁₄PEG2000, wherein the composition is used to synthesize a particle.

In certain embodiments, the composition comprises about 90 molar percent of conjugated lipomer (e.g., 7C1) and about 10 molar percent of C₁₆PEG2000, wherein the composition is used to synthesize a particle.

In certain embodiments, the composition comprises about 90 molar percent of conjugated lipomer (e.g., 7C1) and about 10 molar percent of C₁₈PEG750, wherein the composition is used to synthesize a particle.

In certain embodiments, the composition comprises about 90 molar percent of conjugated lipomer (e.g., 7C1) and about 10 molar percent of C₁₈PEG1000, wherein the composition is used to synthesize a particle.

In certain embodiments, the composition comprises about 90 molar percent of conjugated lipomer (e.g., 7C1) and about 10 molar percent of C₁₈PEG2000, wherein the composition is used to synthesize a particle.

In certain embodiments, the composition comprises about 90 molar percent of conjugated lipomer (e.g., 7C1) and about 10 molar percent of C₁₈PEG3000, wherein the composition is used to synthesize a particle.

In certain embodiments, the composition comprises about 90 molar percent of conjugated lipomer (e.g., 7C1) and about 10 molar percent of C₁₈PEG5000, wherein the composition is used to synthesize a particle.

In certain embodiments, the composition comprises 68 molar percent of conjugated lipomer 7C1 and 32 molar percent of C₁₈PEG5000, wherein the composition is used to synthesize a particle. In certain embodiments, the composition comprises 76 molar percent of conjugated lipomer 7C1 and 24 molar percent of C₁₈PEG5000, wherein the composition is used to synthesize a particle. In certain embodiments, the composition comprises 92 molar percent of conjugated lipomer 7C1 and 8 molar percent of C₁₈PEG5000, wherein the composition is used to synthesize a particle. In certain embodiments, the composition comprises 96 molar percent of conjugated lipomer 7C1 and 4 molar percent of C₁₈PEG5000, wherein the composition is used to synthesize a particle.

In yet another aspect, provided are methods of delivering a biologically active agent to a subject comprising:

administering the composition comprising the biologically active agent to the subject,

wherein the composition includes a particle, wherein the particle comprises:
a biologically active agent;
a conjugated lipomer of Formula (I):

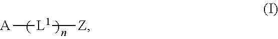

or a pharmaceutically acceptable salt thereof, described herein; and
a lipid-polyethylene glycol (PEG) conjugate of Formula (II):

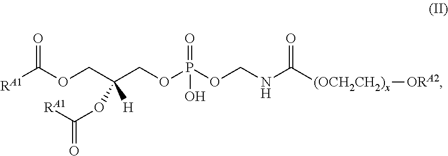

or a pharmaceutically acceptable salt thereof. In certain embodiments, the particle is a nanoparticle. In certain embodiments, the biologically active agent is delivered to the subject in vivo. In certain embodiments, the biologically active agent is delivered to bone marrow in vivo. In certain embodiments, the biologically active agent is delivered to bone marrow cells in vivo. In certain embodiments, the biologically active agent is targeted to bone marrow cells in vivo.

In certain embodiments, the conjugated lipomer of the composition is of Formula (I) described herein. In certain embodiments, the lipid-PEG conjugate of the composition is of Formula (II) described herein. In certain embodiments, the composition is a pharmaceutical composition or a cosmetic composition. In certain embodiments, the agent in the composition is a biologically active agent (e.g., a therapeutic or diagnostic agent). In certain embodiments, the biologically active agent is a small molecule, organometallic compound, nucleic acid, protein, peptide, polynucleotide, metal, targeting agent, an isotopically labeled chemical compound, drug, vaccine, or immunological agent. In certain embodiments, the biologically active agent is a polynucleotide (e.g., DNA or RNA). In certain embodiments, the RNA is RNAi, dsRNA, siRNA, shRNA, miRNA, or antisense RNA. In certain embodiments, the biologically active agent is formulated in the composition by encapsulating the biologically active agent via microfluidic mixing. In certain embodiments, the microfluidic mixing is conducted in a microfluidic device. In certain embodiments, for compositions with the nanoparticle comprising a conjugated lipomer of Formula (I) and a lipid-polyethylene glycol (PEG) conjugate of Formula (II), these compositions of both components were made via microfluidics based synthesis. In certain embodiments, in the microfluidics based synthesis, a conjugated lipomer of Formula (I) and a lipid-polyethylene glycol (PEG) conjugate of Formula (II) complex with nucleic acids via electrostatic interactions to form nanoparticles. In certain embodiments, for a particle comprising a conjugated lipomer of Formula (I) and a lipid-polyethylene glycol (PEG) conjugate of Formula (II), microfluidic devices using chaotic mixing to form particles via electrostatic interactions.

In certain embodiments, the step of administering the biologically active agent to the subject comprises administering the composition intravenously.

The present invention also provides particles comprising both: a conjugated polyethyleneimine (PEI) polymer (also referred to herein as a “conjugated lipomer” or “lipomer”) of Formula (I); and a lipid-polyethylene glycol (PEG) conjugate of Formula (II) described herein. In certain embodiments, the particle is a nanoparticle.

The present invention also provides methods of using the compositions to treat proliferative diseases (e.g., cancers (e.g., bone marrow cancer, leukemia, lymphoma, breast cancer metastasis, leukemia, lymphoma, multiple myeloma, prostate cancer metastasis)), inflammatory diseases, autoinflammatory diseases, autoimmune diseases, genetic diseases (e.g., bone marrow diseases), immunological disorders, and/or hematological disorders in a subject.

The present invention also provides methods of using the compositions for Hematopoietic stem cell transplantation (HSCT). Most patients who would benefit from HSCT are not able to undergo the procedure due to the morbidity associated with the process. Two major areas where HSCT can improve is (i) improving the quantity and purity of HSCs harvested from bone marrow, and (ii) improving gene delivery and correction in HSCs so to better constitute hematopoietic lineages in bone marrow. In certain embodiments, the composition is useful as a delivery system for biologically active agents (e.g., nucleic acids) to key cellular targets in bone marrow.

The present invention also provides methods of using the compositions for treating epithelial and blood cancers (e.g., epithelial and blood cancers that metastasize to and colonize within bone marrow). Patients which have metastasis in bone and bone marrow have poor prognosis. Cancers that colonizes in bone and bone marrow failed to be treated, in part, but not limited to, due to inefficient delivery of drugs to these tissues. In certain embodiments, the composition delivers nucleic acids to bone. In certain embodiments, the composition delivers nucleic acids to bone marrow. In certain embodiments, provided are methods of silencing targets within tumor cells within these tissues, enabling these tumors to now respond to chemo-, radio-, and immunotherapies. In certain embodiments, the nucleic acids silence targets within tumor cells within these tissues. In certain embodiments, the targets within the tumor cells are “undruggable.” In certain embodiments, the cancer is breast cancer, metastatic breast cancer, prostate cancer, multiple myeloma, leukemia, or lymphoma.

Another aspect of the present disclosure relates to kits comprising a container with a particle or composition thereof, as described herein. The kits described herein may include a single dose or multiple doses of the particle or composition. The kits may be useful in a method of the disclosure. In certain embodiments, the kit further includes instructions for using the particle or composition. A kit described herein may also include information (e.g. prescribing information) as required by a regulatory agency, such as the U.S. Food and Drug Administration (FDA).

The details of one or more embodiments of the invention are set forth herein. Other features, objects, and advantages of the invention will be apparent from the Detailed Description, Examples, Figures, and Claims.

### BRIEF DESCRIPTION OF THE DRAWINGS

FIGS. 1A to 1H show a data for composing a bone marrow endothelial cell (BMEC)-targeting nanoparticle. (FIG. 1A) Synthesis scheme of the epoxide-modified polymer-lipid hybrid material. (FIG. 1B) Nanoparticles consisting of lipid hybrid, siRNA and a PEG-lipid conjugate were synthesized via microfluidic mixing. (FIG. 1C) PEG-lipid conjugate parameters varied to form a library of unique nanoparticles for siRNA delivery to BMEC. The best silencing efficiency was obtained with NicheEC-15 (lipid tail length=C₁₈; PEG mol %=10%; PEG MW=5000). (FIG. 1D) Tie2 silencing in femoral bone marrow of C57BL/6 mice 48 h after injection of each nanoparticle containing siRNA against Tie2 (dosage: 1.0 mg/kg; n=5 each). (FIG. 1E) Cryo-TEM micrograph of NicheEC-15. (FIG. 1F) Hydrodynamic diameter of NicheEC-15. (FIG. 1G) Tie2 silencing in bEnd.3 and sorted BMEC 24 hrs after addition of siLuc or siTie2 to the culture medium. Tie2 expression after exposure to siLuc was adjusted to 100% (n=3 each). (FIG. 1H) Confocal microscopy of bEND.3 cells 2 h after adding NicheEC15 to the culture medium. Nuclei were stained with SYTO 13 and cell membranes with Wheat Germ Agglutinin (WGA). In the upper panels the nanoparticle was visualized by siRNA labeled with Alexa Fluor 647 (AF647); in the lower row an unlabeled control siRNA was used. Data are shown as mean±s.e.m.

FIGS. 2A to 2H show in vivo uptake of NicheEC-15 in BMEC. (FIG. 2A) Decline in fluorescence intensity was measured over time following a single injection of NicheEC-15 AF647-siRNA. The in vivo circulation half-life of NicheEC-15 was calculated to be 13.8 min (n=5). (FIG. 2B) Dose response curve of Tie2 silencing in whole bone marrow after in vivo injection of NicheEC-15 or 7C1 nanoparticles encapsulating siTie2 by bDNA assay (dose range 0.01-1.0 mg/kg, n=4-5). (FIG. 2C) Time course of Tie2 expression in whole bone marrow following a single 1.0 mg/kg injection of NicheEC-15 or 7C1 encapsulating siTie2 by bDNA assay. Femurs were harvested 1-18 days post-injection (n=4-5). (FIG. 2D) Intravital microscopy of the skull bone marrow 2 h after injection of NicheEC-15 with AF647-siRNA cargo. The vasculature was stained by injecting PE labeled CD31 and Sca1 antibodies and Osteosense was used to visualize the bone surface. (FIG. 2E) Mice were harvested 2 h after injection of 2 mg/kg NicheEC-15 AF647-siRNA. BMEC were gated as shown in the representative FACS plots. Histograms of endothelial cells (EC) are overlaid with all CD45+ cells and EC of uninjected control mice. (FIG. 2F) Mean fluorescence intensity of uninjected mice, CD45+ leukocytes, BMEC and lung EC 2 hrs after injection of 2 mg/kg AF647-siRNA encapsulated into the 7C1 or NicheEC-15 nanoparticles (n=4-8). (FIG. 2G) The mean fluorescence intensity (MFI) of BMEC was divided by MFI of lung EC for 7C1 and the NicheEC-15. The value is 1.19 for 7C1 and 3.17 for NicheEC-15. (FIG. 2H) Comparison of Tie2 silencing in whole bone marrow and lung after in vivo injection of NicheEC-15 or 7C1 encapsulating siTie2 by a bDNA assay (dose: 1.0 mg/kg, n=4-5). Data are shown as mean±s.e.m. *P<0.05, **P<0.01, ****P<0.0001.

FIGS. 3A to 3M show the effects of siSdf1 silencing on the bone marrow. Mice were injected with 2 mg/kg siSdf1 or control siRNA targeting luciferase (siLuc) on day 0 and day 3 and harvested on day 5. (FIG. 3A) Sdf1 expression in whole bone marrow by qPCR. (FIG. 3B) Sdf1 by ELISA in bone marrow plasma. (FIG. 3C) qPCR for Sdf1 in sorted BMEC. (FIG. 3D) Number of blood LSK by flow cytometry. (FIG. 3E) Colony forming units (CFUs) per ml of whole blood. (FIG. 3F) Representative dot plots for LSK, CMP, MEP and GMP. (FIG. 3G to 3J) Number of LSK, CMP, MEP and GMP in the femoral bone marrow. (FIG. 3K) Representative dot plots of bone marrow neutrophils and monocytes. (FIG. 3L to 3M) Number of monocytes and neutrophils per femur. Data are shown as mean±s.e.m. *P<0.05, **P<0.01, ***P<0.001, ****P<0.0001.

FIGS. 4A to 4F show the release of bone marrow monocytes and neutrophils after siSdf1 treatment. (FIG. 4A) Representative dot plots 2 h after injection. (FIG. 4B) Percentage of neutrophils and monocytes in the blood by flow cytometry. (FIG. 4C) Representative intravital microscopy imaging of Cx3cr1+ cells in the skull bone marrow. Baseline images were taken before injection and cells were tracked by imaging every 10 to 30 min for a total of 2.5 h. The location of cells compared to the baseline and the last imaging after 2.5 h is highlighted with white circles. Circles 3-6 and 8 in the bottom righthand panel mark the area of cells that have left the niche. (FIG. 4D) Percentage of Cx3cr1+ cells in the same area after 2.5 hrs compared to baseline which was adjusted to 100%. (FIG. 4E) Same setup as in (c); neutrophils were labeled with an Ly6g antibody. (FIG. 4F) Similar to (d), the percentage of neutrophils after 2.5 hrs compared to baseline. Data are shown as mean±s.e.m. *P<0.05, **P<0.01, ***P<0.001, ****P<0.0001.

FIGS. 5A to 5I show the effects of siMcp1 treatment during LPS-induced inflammation. (FIG. 5A) Experimental setup. (FIG. 5B) Mcp1 expression in whole bone marrow by qPCR. (FIG. 5C) Mcp1 levels by ELISA in bone marrow plasma. (FIG. 5D) Representative dot plots of bone marrow neutrophils and monocytes. (FIG. 5E) Number of monocytes per femur. (FIG. 5F) Number of Ly6c^{high} monocytes per femur. (FIG. 5G) Representative dot plots of blood neutrophils and monocytes. (FIG. 5H) Number of monocytes per ml of blood. (FIG. 5I) Number of Ly6c^{high} monocytes per ml of blood. Data are shown as mean±s.e.m. *P<0.05, **P<0.01, ****P<0.0001.

FIGS. 6A to 6K show the effects of siMcp1 treatment on inflammatory cells 24 hours after MI. (FIG. 6A) Mcp1 expression in whole bone marrow by qPCR. (FIG. 6B) Mcp1 levels by ELISA in bone marrow plasma. (FIG. 6C) Representative dot plots of bone marrow neutrophils and monocytes. (FIG. 6D) Number of monocytes per femur. (FIG. 6E) Number of Ly6chigh monocytes per femur. (FIG. 6F) Representative dot plots of blood neutrophils and monocytes. (FIG. 6G) Number of monocytes per ml of blood. (FIG. 6H) Number of Ly6chigh monocytes per ml of blood. (FIG. 6I) Representative dot plots of neutrophils and monocytes in the infarct. (FIG. 6J) Number of monocytes per mg infarct tissue. (FIG. 6K) Number of Ly6chigh monocytes per mg infarct tissue. Data are shown as mean±s.e.m. *P<0.05, **P<0.01.

FIGS. 7A to 7C show the siRNAs screen for Sdf1 gene silencing. (FIG. 7A) Sdf1 gene expression in Hepa1-6 cells after treatment with 500 pM of each of 24 different siRNAs for 24 hrs (n=4 each). Results with target-specific siRNAs were normalized to mean activity of two unspecific control siRNAs. The best four siRNAs are highlighted with arrows. (FIG. 7B) Sdf1 knockdown dose response curves in Hepa1-6 after treatment with XD-5171, XD-5173, XD-5180 and XD-5181 siRNAs for 24 hrs (n=4). The best siRNA (XD-5171) which was used for subsequent experiments is highlighted with a box. (FIG. 7C) Sdf1 silencing in endothelial cells in vitro (doses 1-60 nM). bEnd.3 cells were treated with NicheEC-15 containing the XD-5171 siRNA for 24 hrs prior to qPCR analysis (n=4-5 each). Data are shown as mean±s.e.m.

FIGS. 8A to 8C show the siRNAs screen for Mcp1 gene silencing. (FIG. 8A) Mcp1 gene expression in Hepa1-6 cells after treatment with 500 pM of each of 24 different siRNAs for 24 hrs (n=4 each). Results with target-specific siRNAs were normalized to mean activity of two unspecific control siRNAs. The best four siRNAs are highlighted with arrows. (FIG. 8B) Mcp1 knockdown dose response curves in Hepa1-6 after treatment with XD-5136, XD-5137, XD-5143 and XD-5146 siRNAs for 24 hrs (n=4). The best siRNA (XD-5137), which was used for subsequent experiments, is highlighted with a box. (FIG. 8C) Mcp1 silencing in endothelial cells in vitro (doses 1-60 nM). bEND.3 cells were treated with NicheEC-15 containing the XD-5137 siRNA for 24 hrs prior to qPCR analysis (n=4-5 each). Data are shown as mean±s.e.m.

FIG. 9 shows sequences of siRNA. Capital letters represent unmodified nucleotides; lower case letters represent 2′-OCH₃ modified nucleotides. Top to bottom, and left to right, the sequences in this figure correspond to SEQ ID NOs: 3-26.

FIG. 10 lists the components of the nanoparticles of FIG. 1D.

### DEFINITIONS

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, “alkyl” refers to a radical of a straight-chain or branched saturated hydrocarbon group having from 1 to 50 carbon atoms (“C₁₋₅₀ alkyl”). In some embodiments, an alkyl group has 1 to 40 carbon atoms (“C₁₋₄₀ alkyl”). In some embodiments, an alkyl group has 1 to 30 carbon atoms (“C₁₋₃₀ alkyl”). In some embodiments, an alkyl group has 1 to 20 carbon atoms (“C₁₋₂₀ alkyl”). In some embodiments, an alkyl group has 1 to 20 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted C₁₋₅₀ alkyl. In certain embodiments, the alkyl group is a substituted C₁₋₅₀ alkyl.

The term “heteroalkyl,” as used herein, refers to an alkyl group, as defined herein, which further comprises 1 or more (e.g., 1 to 25) heteroatoms (e.g., oxygen, sulfur, nitrogen, boron, silicon, phosphorus) included in the parent chain. In certain embodiments, the heteroalkyl group is an unsubstituted C₁₋₅₀ heteroalkyl. In certain embodiments, the heteroalkyl group is a substituted C₁₋₅₀ heteroalkyl.

As used herein, “alkenyl” refers to a radical of a straight-chain or branched hydrocarbon group having from 2 to 50 carbon atoms and one or more carbon-carbon double bonds (“C₂₋₅₀ alkenyl”). In some embodiments, an alkenyl group has 2 to 40 carbon atoms (“C₂₋₄₀ alkenyl”). In some embodiments, an alkenyl group has 2 to 30 carbon atoms (“C₂₋₃₀ alkenyl”). In some embodiments, an alkenyl group has 2 to 20 carbon atoms (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₅₀ alkenyl. In certain embodiments, the alkenyl group is a substituted C₂₋₅₀ alkenyl.

The term “heteroalkenyl,” as used herein, refers to an alkenyl group, as defined herein, which further comprises 1 or more (e.g., 1 to 25) heteroatoms (e.g., oxygen, sulfur, nitrogen, boron, silicon, phosphorus) included in the parent chain. In certain embodiments, the heteroalkenyl group is an unsubstituted C₂₋₅₀ heteroalkenyl. In certain embodiments, the heteroalkenyl group is a substituted C₂₋₅₀ heteroalkenyl.

As used herein, “alkynyl” refers to a radical of a straight-chain or branched hydrocarbon group having from 2 to 50 carbon atoms and one or more carbon-carbon triple bonds (“C₂₋₅₀ alkynyl”). In some embodiments, an alkynyl group has 2 to 40 carbon atoms (“C₂₋₄₀ alkynyl”). In some embodiments, an alkynyl group has 2 to 30 carbon atoms (“C₂₋₃₀ alkynyl”). In some embodiments, an alkynyl group has 2 to 20 carbon atoms (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₅₀ alkynyl. In certain embodiments, the alkynyl group is a substituted C₂₋₅₀ alkynyl.

The term “heteroalkynyl,” as used herein, refers to an alkynyl group, as defined herein, which further comprises 1 or more (e.g., 1 to 25) heteroatoms (e.g., oxygen, sulfur, nitrogen, boron, silicon, phosphorus) included in the parent chain. In certain embodiments, the heteroalkynyl group is an unsubstituted C₂₋₅₀ heteroalkynyl. In certain embodiments, the heteroalkynyl group is a substituted C₂₋₅₀ heteroalkynyl.

As used herein, “carbocyclyl” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₀ cycloalkyl. In certain embodiments, the cycloalkyl group is a substituted C₃₋₁₀ cycloalkyl.

As used herein, “heterocyclyl” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, thiorenyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

As used herein, “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

As used herein, “heteroaryl” refers to a radical of a 5-14 membered monocyclic or polycyclic (e.g., bicyclic or tricyclic) 4n+2 aromatic ring system (e.g., having 6, 10, or 14π electrons shared in a cyclic array) having ring carbon atoms and 1-4 ring heteroatoms provided in the aromatic ring system, wherein each heteroatom is independently selected from nitrogen, oxygen and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

As used herein, the term “partially unsaturated” refers to a ring moiety that includes at least one double or triple bond. The term “partially unsaturated” is intended to encompass rings having multiple sites of unsaturation, but is not intended to include aromatic groups (e.g., aryl or heteroaryl moieties) as herein defined.

Alkyl, alkenyl, alkynyl, heteroalkyl, heteroalkenyl, heteroalkynyl, carbocyclyl, heterocyclyl, aryl, and heteroaryl groups, as defined herein, are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, 13 OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, —NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{cc}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C_{1-6 alkyl})₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb} )₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

As used herein, the term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═O)SR^{aa}, —SC(═O)OR^{aa}, and —SC(═O)R^{aa}, wherein R^{aa} and R^{cc} are as defined herein.

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

As used herein, the term, “amino” refers to the group —NH₂. The term “substituted amino,” by extension, refers to a monosubstituted amino, a disubstituted amino, or a trisubstituted amino, as defined herein.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

As used herein, the term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

As used herein, the term “sulfonyl” refers to a group selected from —SO₂N(R^{bb})₂, —SO₂R^{aa}, and —SO₂OR^{aa}, wherein R^{aa} and R^{bb} are as defined herein.

As used herein, the term “silyl” refers to the group —Si(R^{aa})₃, wherein R^{aa} is as defined herein.

As used herein, the term “acyl” refers a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

As used herein, the term “halo” or “halogen” refers to fluorine (fluoro, —F), chlorine (chloro, —Cl), bromine (bromo, —Br), or iodine (iodo, —I).

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

As used herein, a “counterion” is a negatively charged group associated with a positively charged quarternary amine in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2- sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

These and other exemplary substituents are described in more detail in the Detailed Description, the Examples and in the claims. The invention is not intended to be limited in any manner by the above exemplary listing of substituents.

As used herein, a “polymer” refers to a compound comprising at least 3 (e.g., at least 4, 5, 10, 20, 30, 40, 50, 60, 70, 80, 90, 100, etc.) repeating structural units covalently bound together.

As used herein, an “organic molecule” is a molecule comprising carbon, as defined herein. The organic molecule may also comprise a metal. In this instance, the organic molecule is also referred to as an “organometallic compound.”

As used herein, an “inorganic molecule” is a molecule which comprises elements other than carbon, and encompasses large inorganic molecules and small inorganic molecules, as defined herein. If an inorganic molecule comprises a transition metal, it is also referred to herein as a “metal.”

As used herein, the term “salt” or “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, sulfonate and aryl sulfonate. Further pharmaceutically acceptable salts include salts formed from the quarternization of an amine using an appropriate electrophile, e.g., an alkyl halide, to form a quarternized alkylated amino salt.

As used herein, use of the phrase “at least one instance” refers to one instance, but also encompasses more than one instance, e.g., for example, from 1 instance to 50 instances.

As used herein, use of the phrase “molar mass averages” refers to different average values (e.g., Mₙ, M_{w}, Mᵥ and M_{z}) can be defined depending upon the statistical method that is applied. The weighted mean can be taken with the weight fraction, the mole fraction, or the volume fraction (see, e.g., R. J. Young and P. A. Lovell, Introduction to Polymers, 1991, incorporated herein by reference).

As used herein, use of the term “dispersity” refers to dispersity (D), which is a measure of the distribution of molecular mass in a given polymer sample and is calculated by dividing the weight average molar mass (M_{w}) by the number average molar mass (Mₙ). The dispersity of a given sample can have a value equal to or greater than 1. As the polymer chains approach uniform chain length, the dispersity approaches unity (1). The dispersity of a polymer can be modified, for example, using polymer fractionation (e.g., preparative SEC, Baker-Williams fractionation, continuous spin fractionation), or modifying the work-up procedure (e.g., by partially dissolving a polymer, an insoluble high molar mass fraction may be filtered off resulting in a large reduction in M_{w} and a small reduction in Mₙ, thus reducing polydispersity).

As used herein, use of the phrase “number average molar mass” or “Mₙ” is defined as the total weight of a sample, divided by the number of molecules in the sample, and is calculated as follows:

$${M}_{n} = \frac{\sum {M}_{i} {N}_{i}}{\sum {N}_{i}} ,$$

where Mₙ is the sum (Σ) of Mᵢ (mass of each molecule in a sample) multiplied by Nᵢ (the number of molecules in the sample), which is divided by the sum (Σ) of the number of molecules in the sample.

As used herein, use of the term “weight average molar mass” or “M_{w}” is defined as the sum (Σ) of of (the square of Mᵢ multipled by Nᵢ (the number of molecules in the sample)), divided by the sum (Σ) of Mᵢ (mass of each molecule in a sample) multiplied by Nᵢ (the number of molecules in the sample), which is is calculated as follows:

$${M}_{W} = \frac{\sum {M}_{i}^{2} {N}_{i}}{\sum {M}_{i} {N}_{i}} .$$

As used herein, use of the phrase “Z average molar mass” or “M_{Z}” is defined as the sum (Σ) of (the cube of Mᵢ multipled by Nᵢ (the number of molecules in the sample)), divided by the sum (Σ) of (the square of Mᵢ (mass of each molecule in a sample) multiplied by Nᵢ (the number of molecules in the sample)), which is is calculated as follows:

$${M}_{Z} = \frac{\sum {M}_{i}^{3} {N}_{i}}{\sum {M}_{i}^{2} {N}_{i}} .$$

As used herein, use of the phrase “viscosity average molar m” or “M_{V}” is defined as

$${M}_{V} = {[ \frac{\sum {M}_{i}^{1 + a} {N}_{i}}{\sum {M}_{i} {N}_{i}} ]}^{1 / a} ,$$

wherein “a” is the exponent in the Mark-Houwink equation.

As used herein, use of the term “lipid” may refer to fats, fatty acids, waxes, phospholipids, or steroids which are soluble in nonpolar organic solvents (e.g., ether, chloroform, acetone, and benzene) and generally insoluble in water. In certain embodiments, the lipid is a fat. In certain embodiments, the lipid is a phospholipid.

As used herein, use of the term “subject” to which administration is contemplated refers to, but is not limited to, humans (i.e., a male or female of any age group, e.g., a pediatric subject (e.g, infant, child, adolescent) or adult subject (e.g., young adult, middle-aged adult or senior adult)) and/or other non-human animals, for example mammals (e.g., primates (e.g., cynomolgus monkeys, rhesus monkeys); commercially relevant mammals such as cattle, pigs, horses, sheep, goats, cats, and/or dogs), birds (e.g., commercially relevant birds such as chickens, ducks, geese, and/or turkeys), reptiles, amphibians, and fish. In certain embodiments, the non-human animal is a mammal. The non-human animal may be a male or female and at any stage of development. A non-human animal may be a transgenic animal.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

As used herein, use of the phrase the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

### DETAILED DESCRIPTION OF CERTAIN EMBODIMENTS OF THE INVENTION

The present invention provides compositions (e.g., nanoparticles) comprising conjugated lipomers of Formula (I); and a lipid-polyethylene glycol (PEG) conjugate of Formula (II); and methods of use (e.g., delivering biologically active agents (e.g., nucleic acids) to a subject (e.g. bone marrow of a subject in vivo)). In certain embodiments, provided is a particle comprising conjugated lipomers of Formula (I); and a lipid-polyethylene glycol (PEG) conjugate of Formula (II). In certain embodiments, the particle is a nanoparticle.

In one aspect, provided is a composition comprising a particle, wherein the particle comprises a conjugated lipomer of Formula (I); and a lipid-polyethylene glycol (PEG) conjugate of Formula (II). In certain embodiments, the particle is a nanoparticle. In certain embodiments, the composition comprising a particle comprises a conjugated lipomer of Formula (I); and a lipid-polyethylene glycol (PEG) conjugate of Formula (II), wherein: each instance of R^{A1} is independently unsubstituted C₆-C₂₀ alkyl; R^{A2} is substituted or unsubstituted alkyl; and x is an integer between 15 to 135, inclusive.

In certain embodiments, the conjugated lipomer is of Formula (I):

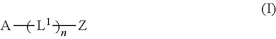

or a pharmaceutically acceptable salt thereof; wherein:

each instance of L¹ is independently selected from the formulae:

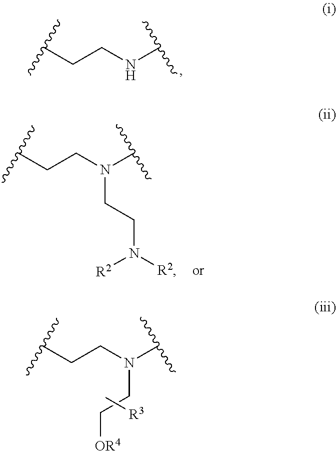

provided that at least one L¹ is selected from formula (iii);

n is an integer between 3 to 45, inclusive;

each instance of R² is independently hydrogen; acyl; silyl; sulfonyl; an amino protecting group; substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; substituted or unsubstituted carbocyclyl; substituted or unsubstituted heterocyclyl; substituted or unsubstituted aryl; substituted or unsubstituted heteroaryl; a substituted or unsubstituted polyethyleneimine; or a group of the formula (iii′):

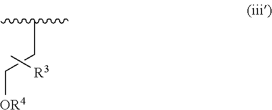

or the two R² groups are joined to form a substituted or unsubstituted heterocyclyl;

each instance of R³ is independently substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; substituted or unsubstituted carbocyclyl; substituted or unsubstituted heterocyclyl; substituted or unsubstituted aryl; substituted or unsubstituted heteroaryl; or a hydrophilic polymer;

each instance of R⁴ is independently hydrogen, acyl; silyl; a hydroxyl protecting group; substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; substituted or unsubstituted carbocyclyl; substituted or unsubstituted heterocyclyl; substituted or unsubstituted aryl; or substituted or unsubstituted heteroaryl;

A is —N(R⁵)₂, wherein each instance of R⁵ is independently hydrogen; acyl; silyl; sulfonyl; an amino protecting group; substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; substituted or unsubstituted carbocyclyl; substituted or unsubstituted heterocyclyl; substituted or unsubstituted aryl; substituted or unsubstituted heteroaryl; or a group of the formula (iii′):

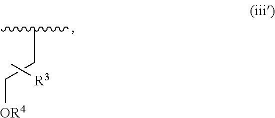

or two R⁵ groups are joined to form a substituted or unsubstituted heterocyclyl; and

Z is hydrogen; acyl; silyl; sulfonyl; an amino protecting group; substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; substituted or unsubstituted carbocyclyl; substituted or unsubstituted heterocyclyl; substituted or unsubstituted aryl; substituted or unsubstituted heteroaryl, or a group of the formula (iii′):

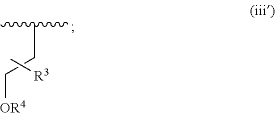

or Z and the nitrogen atom to which it is attached form a substituted or unsubstituted heterocyclyl group.

In certain embodiments, the conjugated lipomer of Formula (I) comprises a polyethyleneimine of molecular weight that is about 650 g/mol or lower. In certain embodiments, the conjugated lipomer of Formula (I) comprises a polyethyleneimine of molecular weight that is about 600 g/mol or lower. In certain embodiments, the conjugated lipomer of Formula (I) comprises a polyethyleneimine of molecular weight that is between about 600 g/mol to about 650 g/mol, about 500 g/mol to about 600 g/mol, between about 400 g/mol to about 500 g/mol, or between about 300 g/mol to about 400 g/mol. In certain embodiments, the conjugated lipomer of Formula (I) comprises a polyethyleneimine of molecular weight that is about 550 g/mol or lower, about 500 g/mol or lower, about 450 g/mol or lower, about 400 g/mol or lower, about 350 g/mol or lower, or about 300 g/mol or lower.

In certain embodiments, each instance of L¹ is independently selected from the formulae:

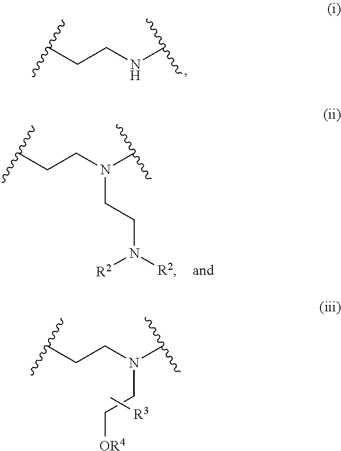

provided that at least one L¹ is selected from formula (iii).

In certain embodiments, the conjugated lipomer of Formula (I) comprises at least one instance of L¹ of the formula (i):

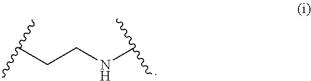 [SMILES: *CCN*]

In certain embodiments, the conjugated lipomer of Formula (I) comprises at least one instance of L¹ of the formula (ii):

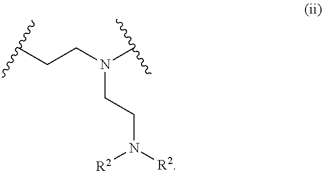

In certain embodiments, the conjugated lipomer of Formula (I) comprises at least one instance of L¹ of the formula:

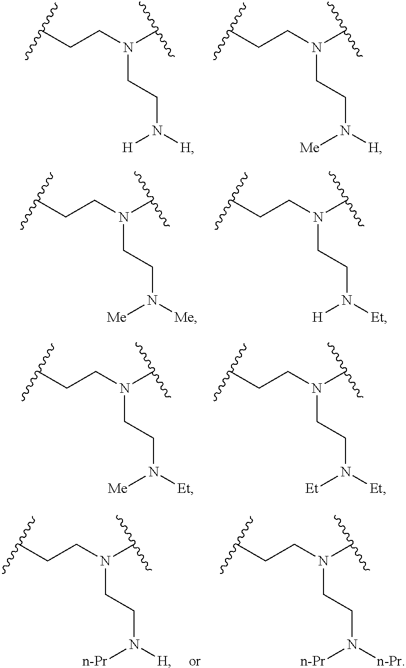

As generally defined above, each instance of R² is independently hydrogen; acyl; silyl; sulfonyl; an amino protecting group; substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; substituted or unsubstituted carbocyclyl; substituted or unsubstituted heterocyclyl; substituted or unsubstituted aryl; substituted or unsubstituted heteroaryl; a substituted or unsubstituted polyethyleneimine; or a group of the formula (iii′); or the two R² groups are joined to form a substituted or unsubstituted heterocyclyl.

In certain embodiments, each instance of R² is independently hydrogen; acyl; silyl; sulfonyl; an amino protecting group; substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; a substituted or unsubstituted polyethyleneimine; or a group of the formula (iii′); or the two R² groups are joined to form a substituted or unsubstituted heterocyclyl. In certain embodiments, at least one instance of R² is hydrogen. In certain embodiments, at least one instance of R² is substituted or unsubstituted C₁₋₆ alkyl. In certain embodiments, at least one instance of R² is methyl. In certain embodiments, at least one instance of R² is ethyl. In certain embodiments, at least one instance of R² is propyl. In certain embodiments, at least one instance of R² is butyl.

In certain embodiments, each instance of R² is independently hydrogen; substituted or unsubstituted alkyl; substituted or unsubstituted heteroalkyl; a substituted or unsubstituted polyethyleneimine; or a group of the formula (iii′); or the two R² groups are joined to form a substituted or unsubstituted heterocyclyl.

In certain embodiments, each instance of R² is independently hydrogen; a substituted or unsubstituted polyethyleneimine; or a group of the formula (iii′):

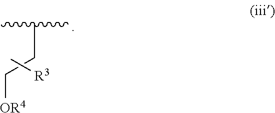

In certain embodiments, at least one R² is a substituted or unsubstituted polyethyleneimine.

In certain embodiments, the conjugated lipomer of Formula (I) comprises at least one instance of L¹ of the formula (iii):

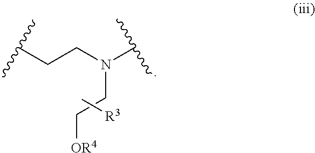

In certain embodiments, at least one instance of L¹ is of the formula (iii-a):

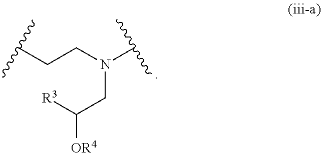

In certain embodiments, at least one instance of L¹ is of the formula:

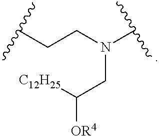

As generally defined within, n is an integer of between 3 to 45, inclusive. In certain embodiments, n is an integer of between 3 to 45, between 5 to 45, between 7 to 45, between 9 to 45, between 10 to 45, between 11 to 45, between 12 to 45, between 13 to 45, between 14 to 45, between 5 to 40, between 5 to 35, between 5 to 30, between 5 to 25, between 5 to 20, between 5 to 15, between 10 to 20, between 10 to 15, or between 40 to 45, inclusive. In certain embodiments, n is an integer between 5 to 10, inclusive.In certain embodiments, n is an integer between 7 to 10, inclusive. In certain embodiments, n is 7, 8, 9, or 10. In certain embodiments, n is 6. In certain embodiments, n is 7. In certain embodiments, n is 8. In certain embodiments, n is 9. In certain embodiments, n is 10. In certain embodiments, n is 14. In certain embodiments, n is 43.

As generally defined above, each instance of R³ is independently substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; substituted or unsubstituted carbocyclyl; substituted or unsubstituted heterocyclyl; substituted or unsubstituted aryl; substituted or unsubstituted heteroaryl; or a hydrophilic polymer.

In certain embodiments, each instance of R³ is independently substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; or a hydrophilic polymer.

In certain embodiments, each instance of R³ is independently substituted or unsubstituted alkyl; substituted or unsubstituted heteroalkyl; or a hydrophilic polymer.

In certain embodiments, at least one instance of R³ is substituted or unsubstituted alkyl. In certain embodiments, at least one instance of R³ is substituted or unsubstituted C₁₋₅₀ alkyl. In certain embodiments, at least one instance of R³ is C₆-C₁₆ substituted or unsubstituted alkyl. In certain embodiments, at least one instance of R³ is C₆-C₁₂ substituted or unsubstituted alkyl. In certain embodiments, at least one instance of R³ is C₁₂ unsubstituted alkyl. In certain embodiments, all instances of R³ are C₁₂ unsubstituted alkyl. In certain embodiments, at least one instance of R³ is substituted or unsubstituted C₈₋₅₀ alkyl. In certain embodiments, at least one instance of R³ is substituted or unsubstituted C₈₋₄₀ alkyl. In certain embodiments, at least one instance of R³ is substituted or unsubstituted C₈₋₃₀ alkyl. In certain embodiments, at least one instance of R³ is substituted or unsubstituted C₈₋₂₀ alkyl.

In certain embodiments, at least one instance of R³ is an unsubstituted alkyl. Exemplary unsubstituted alkyl groups include, but are not limited to, —CH₃, —C₂H₅, —C₃H₇, —C₄H₉, —C₅H₁₁, —C₆H₁₃, —C₇H₁₅, —C₈H₁₇, —C₉H₁₉, —C₁₀H₂₁, —C₁₁H₂₃, —C₁₂H₂₅, —C₁₃H₂₇, —C₁₄H₂₉, —C₁₅H₃₁, —C₁₆H₃₃, —C₁₇H₃₅, —C₁₈H₃₇, —C₁₉H₃₉, and —C₂₀H₄₁.

In certain embodiments, at least one instance of R³ is substituted alkyl. For example, in certain embodiments, at least one instance of R³ is an alkyl substituted with one or more fluorine substituents. Exemplary substituted alkyl groups include, but are not limited to:

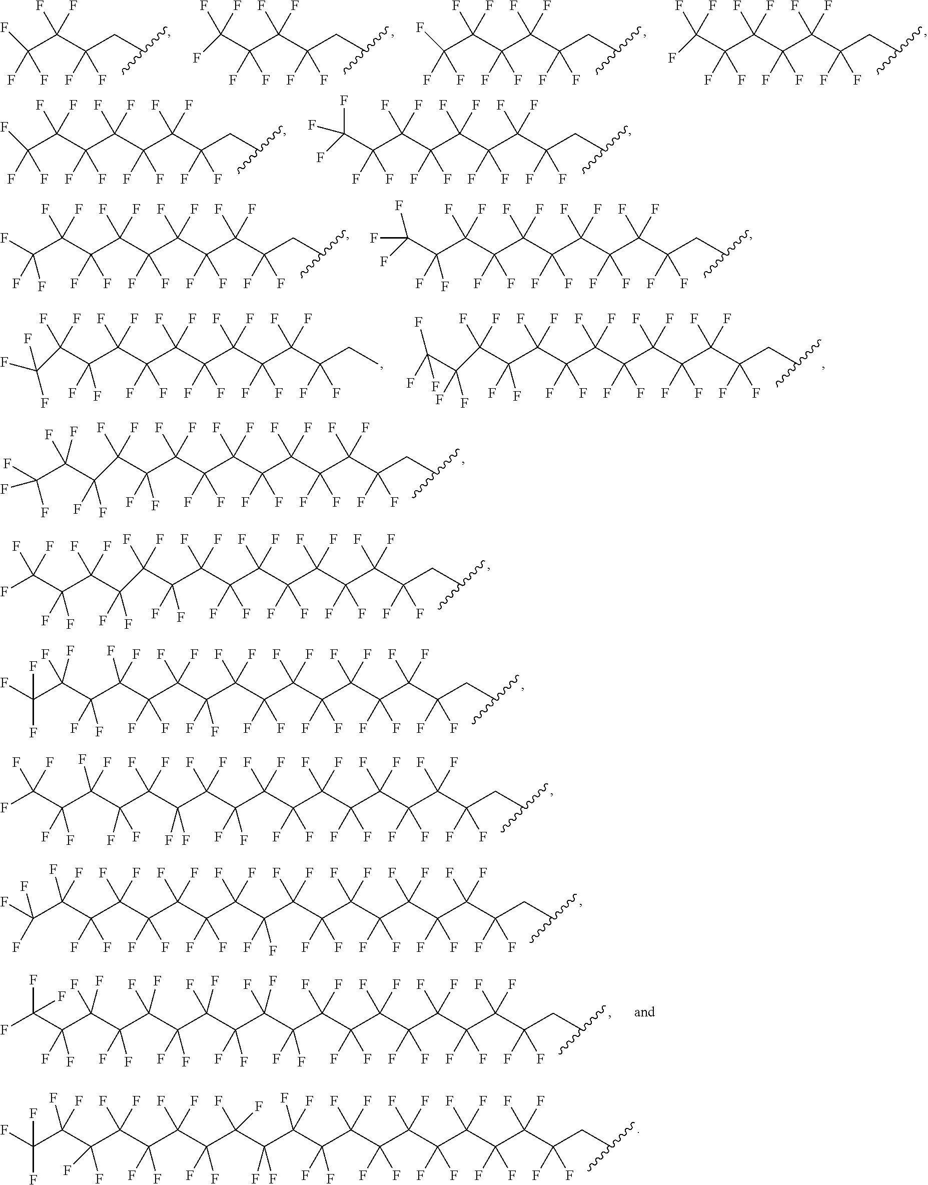

In certain embodiments, at least one instance of R³ is substituted or unsubstituted alkenyl. In certain embodiments, at least one instance of R³ is substituted or unsubstituted C₂₋₅₀alkenyl. In certain embodiments, at least one instance of R³ is substituted or unsubstituted C₈₋₅₀ alkenyl. In certain embodiments, at least one instance of R³ is substituted or unsubstituted C₈₋₄₀ alkenyl. In certain embodiments, at least one instance of R³ is substituted or unsubstituted C₈₋₃₀ alkenyl. In certain embodiments, at least one instance of R³ is substituted or unsubstituted C₈₋₂₀ alkenyl. In certain embodiments, at least one instance of R³ is a substituted C₈₋₂₀ alkenyl.

In certain embodiments, at least one instance of R³ is an unsubstituted alkenyl. Exemplary unsubstituted alkenyl groups as R³ include, but are not limited to:

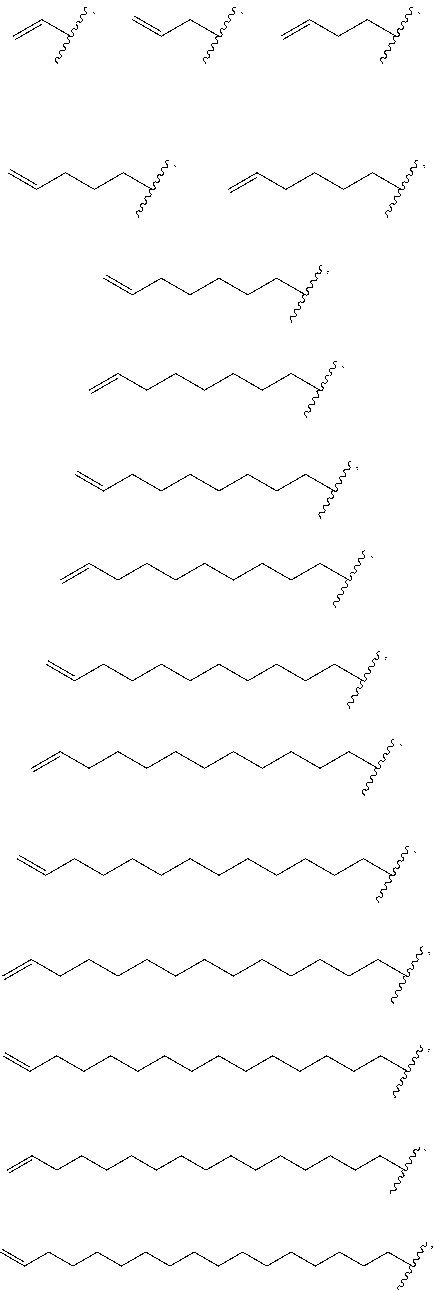

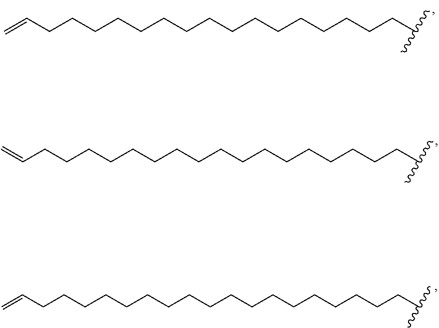 [SMILES: *CCCCCCCCCCCCCCCCC=C ; *CCCCCCCCCCCCCCCCCC=C ; *CCCCCCCCCCCCCCCCCCC=C]

Myristoleic —(CH₂)₇CH═CH(CH₂)₃CH₃,
Palmitoliec —(CH₂)₇CH═CH(CH₂)₅CH₃,
Sapienic —(CH₂)₄CH═CH(CH₂)₈CH₃,
Oleic —(CH₂)₇CH═CH(CH₂)₇CH₃,
Linoleic —(CH₂)₇CH═CHCH₂CH═CH(CH₂)₄CH₃,
α-Linolenic —(CH₂)₇CH═CHCH₂CH═CHCH₂CH═CHCH₂CH₃,
Arachinodonic —(CH₂)₃CH═CHCH₂CH═CHCH₂CH═CHCH₂CH═CH(CH₂)₄CH₃,
Eicosapentaenoic —(CH₂)₃CH═CHCH₂CH═CHCH₂CH═CHCH₂CH═CHCH₂CH═CHCH₂CH₃,
Erucic —(CH₂)₁₁CH═CH(CH₂)₇CH₃, and
Docosahexaenoic —(CH₂)₂CH═CHCH₂CH═CHCH₂CH═CHCH₂CH═CHCH₂CH═CHCH₂CH═CH—CH₂CH₃.

In certain embodiments, wherein R³ is defined as a C₆₋₅₀ alkyl or C₆₋₅₀alkenyl groups, such groups encompass lipophilic groups (also referred to as a “lipid tail”). Lipophilic groups comprise a group of molecules that include fats, waxes, oils, fatty acids, and the like. Lipid tails present in these lipid groups can be saturated and unsaturated, depending on whether or not the lipid tail comprises double bonds. The lipid tail can also comprise different lengths, often categorized as medium (i.e., with tails between 7-12 carbons, e.g., C₇₋₁₂ alkyl or C₇₋₁₂ alkenyl), long (i.e., with tails greater than 12 carbons and up to 22 carbons, e.g., C₁₃₋₂₂ alkyl or C₁₃₋₂₂ alkenyl), or very long (i.e., with tails greater than 22 carbons, e.g., C₂₃₋₃₀ alkyl or C₂₃₋₃₀ alkenyl).

In certain embodiments, at least one instance of R³ is substituted or unsubstituted alkynyl. In certain embodiments, at least one instance of R³ is substituted or unsubstituted C₂₋₅₀alkynyl. In certain embodiments, at least one instance of R³ is substituted or unsubstituted C₈₋₅₀ alkynyl. In certain embodiments, at least one instance of R³ is substituted or unsubstituted C₈₋₄₀ alkynyl. In certain embodiments, at least one instance of R³ is substituted or unsubstituted C₈₋₃₀ alkynyl. In certain embodiments, at least one instance of R³ is substituted or unsubstituted C₈₋₂₀ alkynyl. In certain embodiments, at least one instance of R³ is an unsubstituted alkynyl. In certain embodiments, at least one instance of R³ is a substituted alkynyl.

In certain embodiments, at least one instance of R³ is substituted or unsubstituted heteroalkyl. In certain embodiments, at least one instance of R³ is substituted or unsubstituted C₁₋₅₀ heteroalkyl. In certain embodiments, at least one instance of R³ is substituted or unsubstituted C₈₋₅₀ heteroalkyl. In certain embodiments, at least one instance of R³ is substituted or unsubstituted C₈₋₄₀ heteroalkyl. In certain embodiments, at least one instance of R³ is substituted or unsubstituted C₈₋₃₀ heteroalkyl. In certain embodiments, at least one instance of R³ is substituted or unsubstituted C₈₋₂₀ heteroalkyl. In certain embodiments, at least one instance of R³ is a substituted heteroalkyl.

In certain embodiments, at least one instance of R³ is an unsubstituted heteroalkyl. Exemplary unsubstituted heteroalkyl groups as R³ include, but are not limited to:

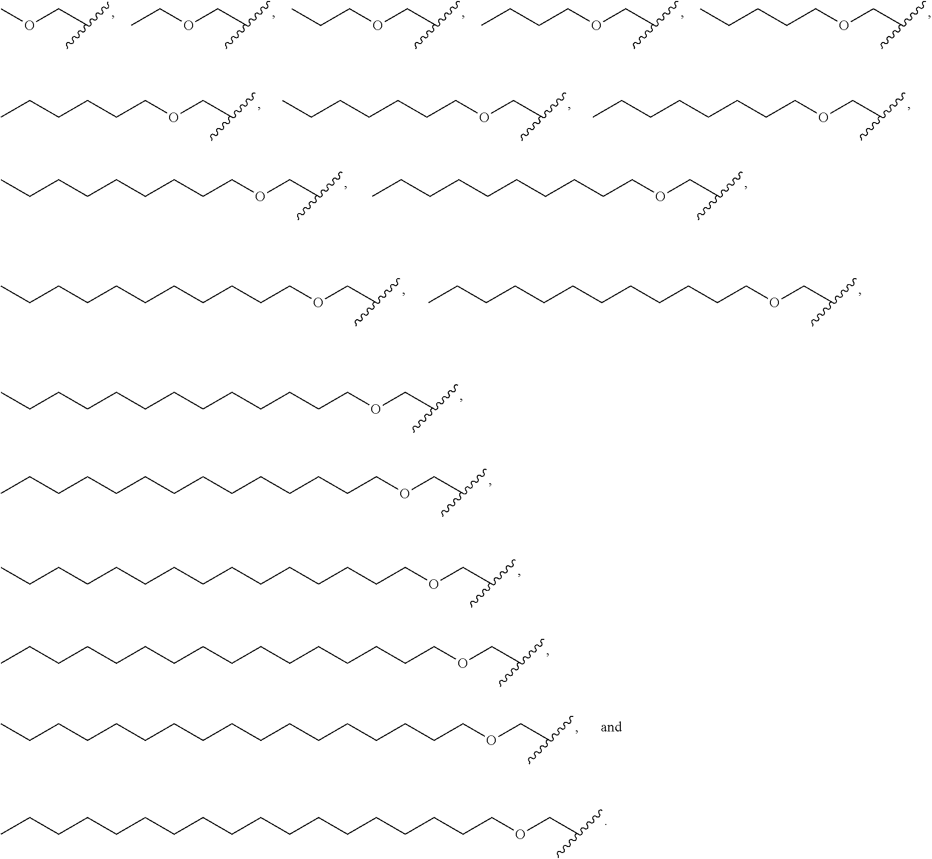

In certain embodiments, at least one instance of R³ is substituted or unsubstituted heteroalkenyl. In certain embodiments, at least one instance of R³ is substituted or unsubstituted C₂₋₅₀ heteroalkenyl. In certain embodiments, at least one instance of R³ is substituted or unsubstituted C₈₋₅₀ heteroalkenyl. In certain embodiments, at least one instance of R³ is substituted or unsubstituted C₈₋₄₀ heteroalkenyl. In certain embodiments, at least one instance of R³ is substituted or unsubstituted C₈₋₃₀ heteroalkenyl. In certain embodiments, at least one instance of R³ is substituted or unsubstituted C₈₋₂₀ heteroalkenyl. In certain embodiments, at least one instance of R³ is a substituted heteroalkenyl. In certain embodiments, at least one instance of R³ is an unsubstituted heteroalkenyl.

In certain embodiments, at least one instance of R³ is substituted or unsubstituted heteroalkynyl. In certain embodiments, at least one instance of R³ is substituted or unsubstituted C₂₋₅₀ heteroalkynyl. In certain embodiments, at least one instance of R³ is substituted or unsubstituted C₈₋₅₀ heteroalkynyl. In certain embodiments, at least one instance of R³ is substituted or unsubstituted C₈₋₄₀ heteroalkynyl. In certain embodiments, at least one instance of R³ is substituted or unsubstituted C₈₋₃₀ heteroalkynyl. In certain embodiments, at least one instance of R³ is substituted or unsubstituted C₈₋₂₀ heteroalkynyl. In certain embodiments, at least one instance of R³ is a substituted heteroalkynyl. In certain embodiments, at least one instance of R³ is an unsubstituted heteroalkynyl.

In certain embodiments, at least one instance of R³ is substituted or unsubstituted carbocyclyl. In certain embodiments, at least one instance of R³ is a substituted carbocyclyl. In certain embodiments, at least one instance of R³ is an unsubstituted carbocyclyl.

In certain embodiments, at least one instance of R³ is substituted or unsubstituted heterocyclyl. In certain embodiments, at least one instance of R³ is a substituted heterocyclyl. In certain embodiments, at least one instance of R³ is an unsubstituted heterocyclyl.

In certain embodiments, at least one instance of R³ is substituted or unsubstituted aryl. In certain embodiments, at least one instance of R³ is an unsubstituted aryl. In certain embodiments, at least one instance of R³ is a substituted aryl.

In certain embodiments, at least one instance of R³ is substituted or unsubstituted heteroaryl. In certain embodiments, at least one instance of R³ is a substituted heteroaryl. In certain embodiments, at least one instance of R³ is an unsubstituted heteroaryl.

In certain embodiments, at least one instance of R³ is hydrophilic polymer. As used herein, a “polymer” refers to a compound comprised of at least 3 (e.g., at least 10, 20, 30, 40, 50, 60, 70, 80, 90, 100, etc.) repeating covalently bound structural units. By extension, a “hydrophilic polymer” is a polymer, as defined herein, further comprising at least one group (e.g., an oxygen, nitrogen, and/or sulfur atom) in the repeating structural unit capable of hydrogen bonding. The hydrophilic polymer is preferably biocompatible (i.e., non-toxic). Exemplary hydrophilic polymers include, but are not limited to, polypeptides (e.g., poly-L-lysine), cellulose polymers (e.g., hydroxyethylcellulose, ethylcellulose, carboxymethylcellulose, methylc cellulose, hydroxypropylmethylcellulose (HPMC)), dextran polymers, polymaleic acid polymers, poly(acrylic acid) polymers, poly(vinylalcohol) polymers, polyvinylpyrrolidone (PVP) polymers, and polyethyleneglycol (PEG) polymers.

In certain embodiments, at least one instance of R³ is hydrophilic polymer. In certain embodiments, the hydrophilic polymer as R³ is a polyethyleneglycol polymer, e.g., of the formula (v):

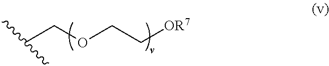

wherein:

R⁷ is hydrogen; acyl; silyl; a hydroxyl protecting group; substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; substituted or unsubstituted carbocyclyl; substituted or unsubstituted heterocyclyl; substituted or unsubstituted aryl; or substituted or unsubstituted heteroaryl; and

v is an integer between 3 to 400, inclusive.

In certain embodiments, R⁷ is hydrogen. In certain embodiments, R⁷ is acyl. In certain embodiments, R⁷ is a hydroxyl protecting group. In certain embodiments, R⁷ is substituted or unsubstituted alkyl. In certain embodiments, R⁷ is a substituted alkyl. In certain embodiments, R⁷ is an unsubstituted alkyl. In certain embodiments, R⁷ is —CH₃ (a “polyethyleneglycol monomethylether” polymer). In certain embodiments, R⁷ is substituted or unsubstituted alkenyl. In certain embodiments, R⁷ is substituted or unsubstituted alkynyl. In certain embodiments, R⁷ is substituted or unsubstituted heteroalkyl. In certain embodiments, R⁷ is substituted or unsubstituted heteroalkenyl. In certain embodiments, R⁷ is substituted or unsubstituted heteroalkynyl. In certain embodiments, R⁷ is substituted or unsubstituted carbocyclyl. In certain embodiments, R⁷ is substituted or unsubstituted heterocyclyl. In certain embodiments, R⁷ is substituted or unsubstituted aryl. In certain embodiments, R⁷ is and substituted or unsubstituted heteroaryl.

In certain embodiments, v is an integer between 3 to 300, 3 to 200, 3 to 100, 3 to 90, 3 to 80, 3 to 70, 3 to 60, 3 to 50, 5 to 50, 10 to 50, 15 to 50, 20 to 50, 20 to 40, 20 to 30, 20 to 25, 30 to 50, and 40 to 50, inclusive. PEG₁₀₀₀ corresponds, on average, to a v of about 22.7, wherein R⁷ is —OCH₃. PEG₂₀₀₀ corresponds, on average, to a v of about 45.4.

In certain embodiments, the number average molar mass (Mn) of the polyethyleneglycol polymer is ≤10,000. In certain embodiments, the number average molar mass (Mn) of the polyethyleneglycol polymer is ≤10,000, ≤9000, ≤8000, ≤7000, ≤6000, ≤5000, ≤4000, ≤3000, or ≤2000. In certain embodiments, the number average molar mass (Mn) of the polyethyleneglycol polymer is between about 100 to about 10,000, inclusive; e.g., between about 100 to about 5000, between about 100 to about 4000, between about 100 to about 3000, between about 100 to about 2500, between about 100 to about 2000, between about 100 to about 1500, between about 100 to about 1000, between about 100 to about 900, between about 100 to about 800, between about 100 to about 700, between about 100 to about 600, between about 100 to about 500, between about 100 to about 400, between about 100 to about 300, between about 100 to about 200, between about 100 to about 1500, between about 2500 to about 10000, between about 2500 to about 9000, between about 2500 to about 8000, between about 2500 to about 7000, between about 2500 to about 6000, between about 2500 to about 5000, between about 2500 to about 4000, or between about 2500 to about 3000, inclusive. In certain embodiments, the number average molar mass (Mn) of the polyethyleneglycol polymer is 1000 (PEG₁₀₀₀). In certain embodiments, the number average molar mass (Mn) of the polyethyleneglycol polymer is 2000 (PEG₂₀₀₀). A 1:1 mixture of PEG₁₀₀₀ and PEG₂₀₀₀ is referred to herein as PEG_{1.5K}.

In certain embodiments, at least one instance of R³ is a hydrophilic polymer, and at least one instance of R³ is a substituted or unsubstituted alkyl.

As used herein, when the group R³ is depicted as bisecting a carbon-carbon bond, e.g., of the group of the formula (iii), it is understood that R³ may be substituted at either carbon.

In certain embodiments, the conjugated lipomer comprises two different R³ groups. For example, in certain embodiments, the conjugated lipomer comprises a mixture of two different R³ groups, wherein the first R³ group is a substituted or unsubstituted alkyl, and the second R³ group is a hydrophilic polymer (e.g., a polyethyleneglycol polymer). In certain embodiments, the conjugated lipomer comprises a mixture of two different R³ groups, wherein the first R³ group is an unsubstituted alkyl, and the second R³ group is a polyethyleneglycol polymer. In certain embodiments, the conjugated lipomer comprises a mixture of two different R³ groups, wherein the first R³ group is selected from the group consisting of —CH₃, —C₂H₅, —C₃H₇, —C₄H₉, —C₅H₁₁, —C₆H₁₃, —C₇H₁₅, —C₈H₁₇, —C₉H₁₉, —C₁₀H₂₁, —C₁₁H₂₃, —C₁₂H₂₅, —C₁₃H₂₇, —C₁₄H₂₉, —C₁₅H₃₁, —C₁₆H₃₃, —C₁₇H₃₅, —C₁₈H₃₇, —C₁₉H₃₉, and —C₂₀H₄₁, and the second R³ group is PEG₁₀₀₀. In certain embodiments, the conjugated lipomer comprises a mixture of two different R³ groups, wherein the first R³ group is selected from the group consisting of —CH₃, —C₂H₅, —C₃H₇, —C₄H₉, —C₅H₁₁, —C₆H₁₃, —C₇H₁₅, —C₈H₁₇, —C₉H₁₉, —C₁₀H₂₁, —C₁₁H₂₃, —C₁₂H₂₅, —C₁₃H₂₇, —C₁₄H₂₉, —C₁₅H₃₁, —C₁₆H₃₃, —C₁₇H₃₅, —C₁₈H₃₇, —C₁₉H₃₉, and —C₂₀H₄₁, and the second R³ group is PEG₂₀₀₀.

In certain embodiments, the conjugated lipomer comprises three different R³ groups. For example, in certain embodiments, the conjugated lipomer comprises a mixture of three different R³ groups, wherein the first R³ group is a substituted or unsubstituted alkyl, the second R³ group is a first hydrophilic polymer (e.g., a polyethyleneglycol polymer, e.g., PEG₁₀₀₀), and the third R³ group is a second hydrophilic polymer (e.g., a different polyethyleneglycol polymer, e.g., PEG₂₀₀₀). In certain embodiments, the conjugated lipomer comprises a mixture of three different R³ groups, wherein the first R³ group is an unsubstituted alkyl, the second R³ group is PEG₁₀₀₀, and the third R³ group is PEG₂₀₀₀. In certain embodiments, the conjugated lipomer comprises a mixture of three different R³ groups, wherein the first R³ group is selected from the group consisting of —CH₃, —C₂H₅, —C₃H₇, —C₄H₉, —C₅H₁₁, —C₆H₁₃, —C₇H₁₅, —C₈H₁₇, —C₉H₁₉, —C₁₀H₂₁, —C₁₁H₂₃, —C₁₂H₂₅, —C₁₃H₂₇, —C₁₄H₂₉, —C₁₅H₃₁, —C₁₆H₃₃, —C₁₇H₃₅, —C₁₈H₃₇, —C₁₉H₃₉, and —C₂₀H₄₁, the second R³ group is PEG₁₀₀₀, and the third R³ group is PEG₂₀₀₀. In certain embodiments a 1:1 mixture of PEG₁₀₀₀ and PEG₂₀₀₀ is used. In this instance, the mixture of the second R³ group and the third R³ group are referred to herein as PEG_{1.5K}.

In certain embodiments, the conjugated polymer comprises more of formula (iii) than of formula (i). For example, in certain embodiments, the ratio of groups of the formulae (i) to (iii) is between about 0:10 to about 9:10, inclusive. In certain embodiments, the ratio of groups of the formulae (i) to (iii) is between about 0:10 to about 9:10; between about 1:10 to about 8:10; between about 1:10 to about 7:10; between about 1:10 to about 6:10; between about 1:10 to about 5:10; or between about 2:10 to about 4:10, inclusive. In certain embodiments, the ratio of groups of the formulae (i) to (iii) is between about 3:10 to about 4:10, inclusive.

Alternatively, in certain embodiments, the conjugated polymer comprises more of formula (i) than of formula (iii). For example, in certain embodiments, the ratio of groups of the formulae (iii) to (i) is between about 0:10 to about 9:10, inclusive. In certain embodiments, the ratio of groups of the formulae (iiii) to (i) is between about 0:10 to about 9:10; between about 1:10 to about 8:10; between about 1:10 to about 7:10; between about 1:10 to about 6:10; between about 1:10 to about 5:10; or between about 2:10 to about 4:10, inclusive. In certain embodiments, the ratio of groups of the formulae (iii) to (i) is between about 3:10 to about 4:10, inclusive.

In certain embodiments, wherein the conjugated lipomer comprises two different R³ groups, the ratio of the second R³ group to the first R³ group is between about 0.01:10 to about 10:10, inclusive. In certain embodiments, the ratio of the second R³ group to the first R³ group is between about 0.02:10 to about 10:10; between about 0.03:10 to about 10:10; between about 0.04:10 to about 10:10; between about 0.05:10 to about 10:10; between about 0.06:10 to about 10:10; between about 0.07:10 to about 10:10; between about 0.08:10 to about 10:10; between about 0.08:10 to about 9:10; between about 0.08:10 to about 8:10; between about 0.08:10 to about 7:10; between about 0.08:10 to about 6:10; between about 0.08:10 to about 5:10; between about 0.08:10 to about 4:10; between about 0.08:10 to about 3:10; between about 0.08:10 to about 2:10; or between about 0.08:10 to about 1:10, inclusive. In certain embodiments, the ratio of the second R³ group to the first R³ group is about 0.1:10.

In certain embodiments, wherein the conjugated lipomer comprises three different R³ groups, the ratio of the sum of the second and third R³ groups to the first R³ group is between about 0.01:10 to about 10:10, inclusive. In certain embodiments, the ratio of the sum of the second and third R³ groups to the first R³ group is 0.02:10 to about 10:10; between about 0.03:10 to about 10:10; between about 0.04:10 to about 10:10; between about 0.05:10 to about 10:10; between about 0.06:10 to about 10:10; between about 0.07:10 to about 10:10; between about 0.08:10 to about 10:10; between about 0.08:10 to about 9:10; between about 0.08:10 to about 8:10; between about 0.08:10 to about 7:10; between about 0.08:10 to about 6:10; between about 0.08:10 to about 5:10; between about 0.08:10 to about 4:10; between about 0.08:10 to about 3:10; between about 0.08:10 to about 2:10; or between about 0.08:10 to about 1:10, inclusive. In certain embodiments, the ratio of the sum of the second and third R³ groups to the first R³ group is about 0.1:10.

Exemplary conjugated lipomers of Formula (I) include, but are not limited to, any of the following LPEI conjugated polymers and BPEI conjugated polymers, or salts thereof, provided in Tables 1 and 2, defining the one or more L₁ groups present along the polymer backbone.

TABLE 1
LPEI conjugated polymers

| | (i) | (iii) | (iii) |
|---|---|---|---|
| 1 | — | 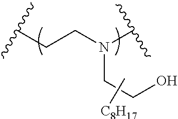 | — |
| 2 | 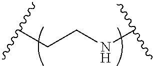 | 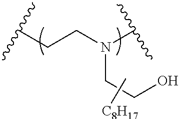 | — |
| 3 | 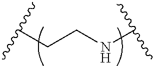 | 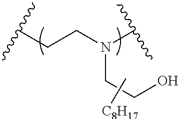 | 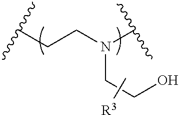 |
| 4 | — | 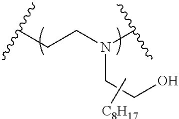 | 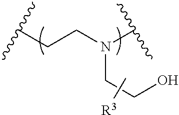 |
| 5 | — | 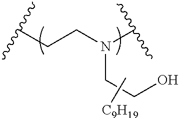 | — |
| 6 | 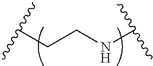 | 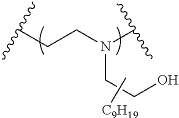 | — |
| 7 | 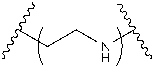 | 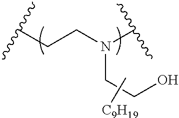 | 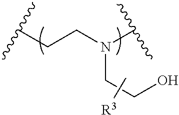 |
| 8 | — | 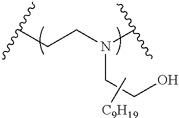 | 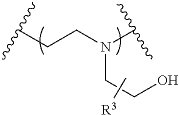 |
| 9 | — | 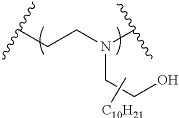 | — |
| 10 | 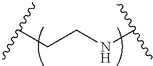 | 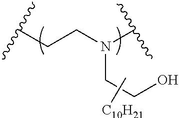 | — |
| 11 | 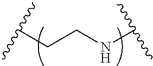 | 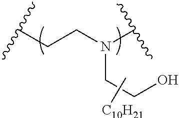 | 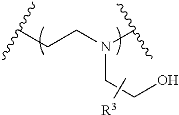 |
| 12 | — | 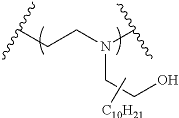 | 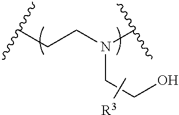 |
| 13 | — | 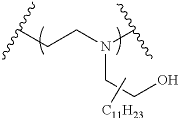 | — |
| 14 | 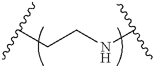 | 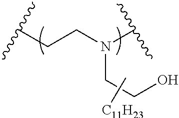 | — |
| 15 | 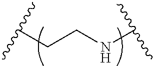 | 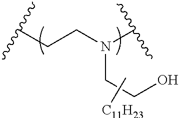 | 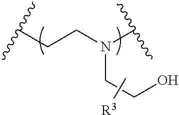 |
| 16 | — | 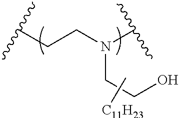 | 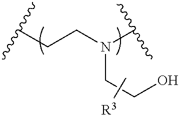 |
| 17 | — | 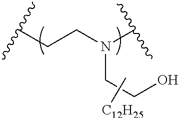 | — |
| 18 | 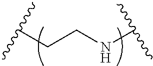 | 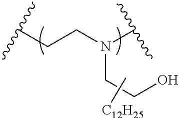 | — |
| 19 | 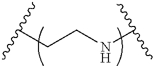 | 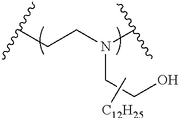 | 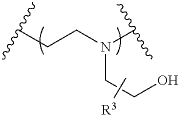 |
| 20 | — | 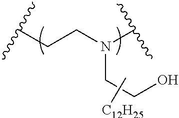 | 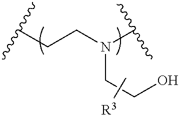 |
| 21 | — | 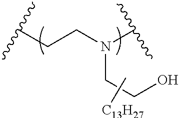 | — |
| 22 | 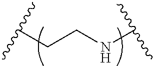 | 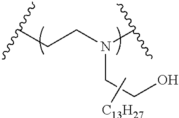 | — |
| 23 | 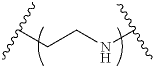 | 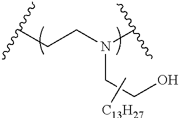 | 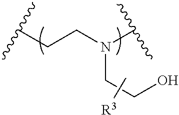 |
| 24 | — | 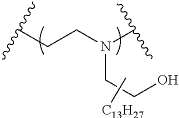 | 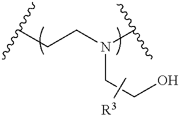 |
| 25 | — | 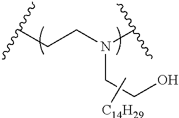 | — |
| 26 | 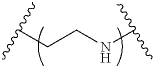 | 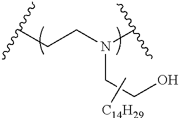 | — |
| 27 | 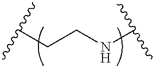 | 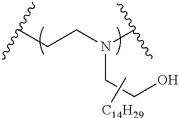 | 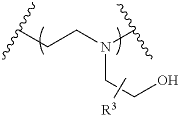 |
| 28 | — | 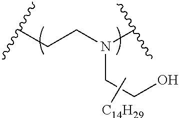 | 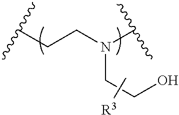 |
| 29 | — | 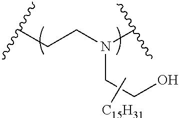 | — |
| 30 | 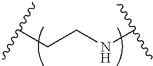 | 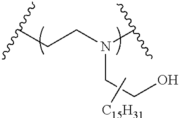 | — |
| 31 | 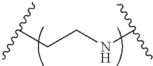 | 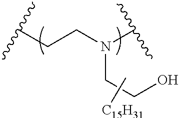 | 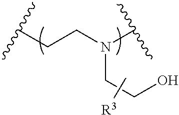 |
| 32 | — | 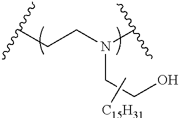 | 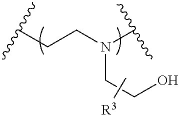 |
| 33 | — | 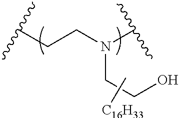 | — |
| 34 | 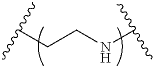 | 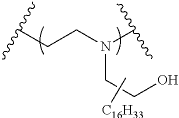 | — |
| 35 | 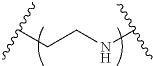 | 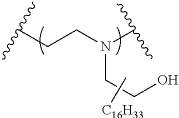 | 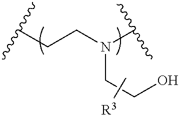 |
| 36 | — | 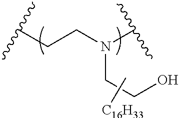 | 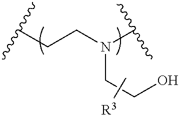 |

TABLE 2
BPEI conjugated polymers

| | (i) | (ii) | (iii) | (iii) |
|---|---|---|---|---|
| 1 | — | 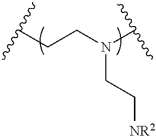 | 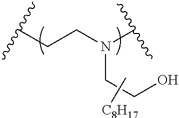 | — |
| 2 | 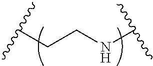 | 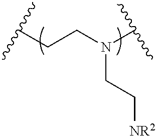 | 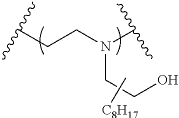 | — |
| 3 | 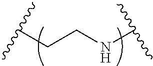 | 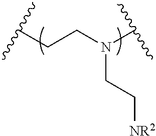 | 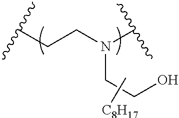 | 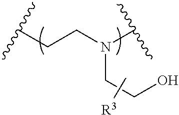 |
| 4 | — | 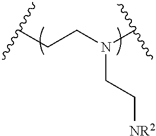 | 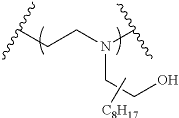 | 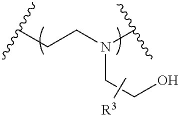 |
| 5 | — | 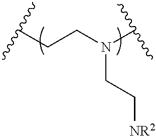 | 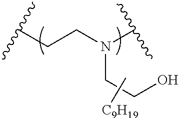 | — |
| 6 | 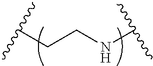 | 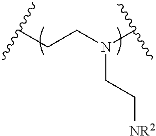 | 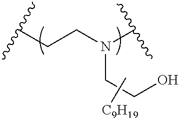 | — |
| 7 | 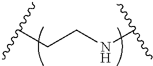 | 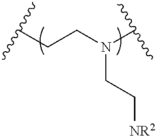 | 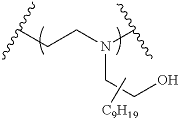 | 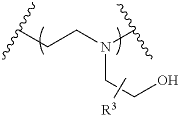 |
| 8 | — | 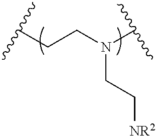 | 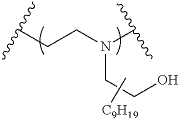 | 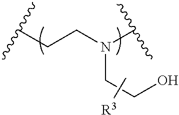 |
| 9 | — | 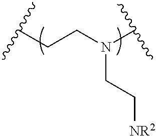 | 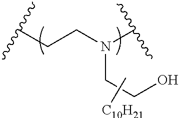 | — |
| 10 | 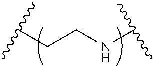 | 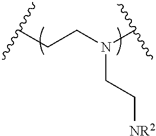 | 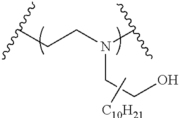 | — |
| 11 | 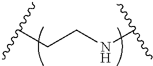 | 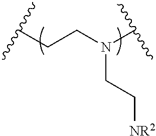 | 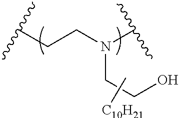 | 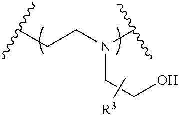 |
| 12 | — | 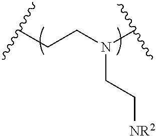 | 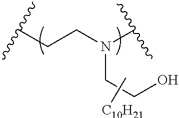 | 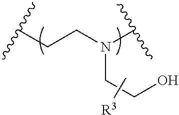 |
| 13 | — | 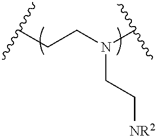 | 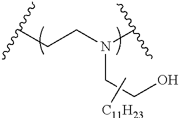 | — |
| 14 | 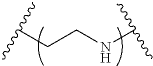 | 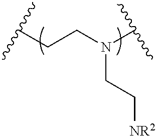 | 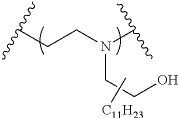 | — |
| 15 | 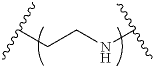 | 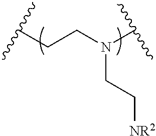 | 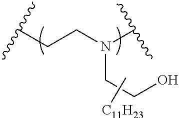 | 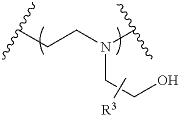 |
| 16 | — | 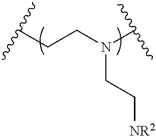 | 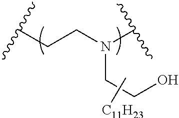 | 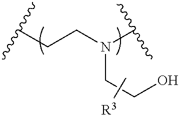 |
| 17 | — | 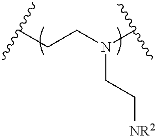 | 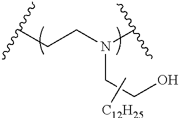 | — |
| 18 | 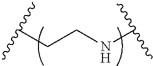 | 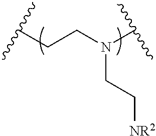 | 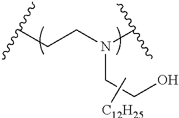 | — |
| 19 | 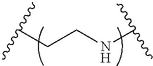 | 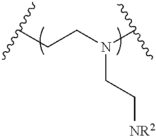 | 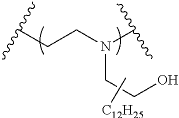 | 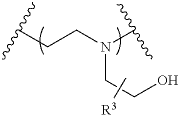 |
| 20 | — | 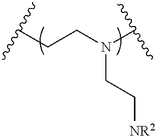 | 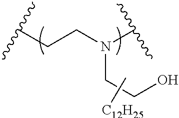 | 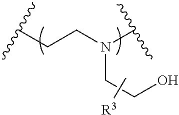 |
| 21 | — | 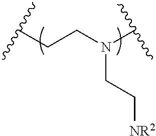 | 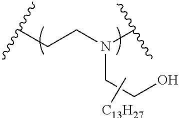 | — |
| 22 | 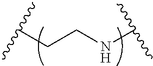 | 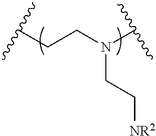 | 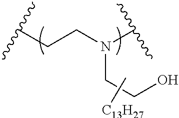 | — |
| 23 | 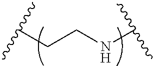 | 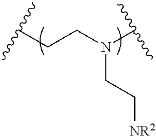 | 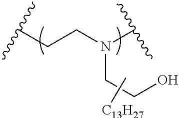 | 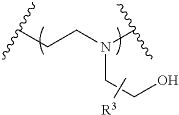 |
| 24 | — | 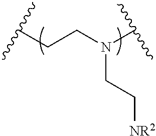 | 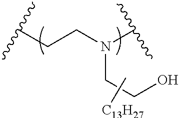 | 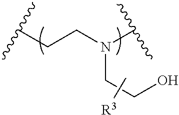 |
| 25 | — | 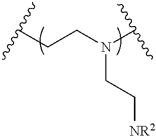 | 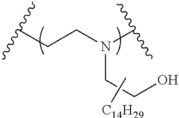 | — |
| 26 | 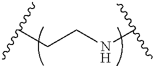 | 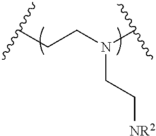 | 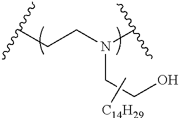 | — |
| 27 | 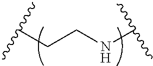 | 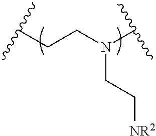 | 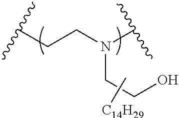 | 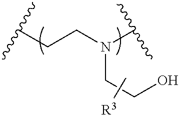 |
| 28 | — | 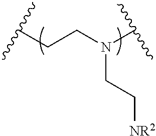 | 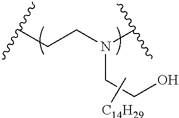 | 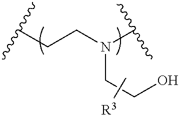 |
| 29 | — | 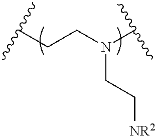 | 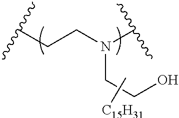 | — |
| 30 | 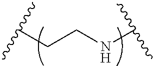 | 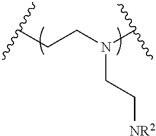 | 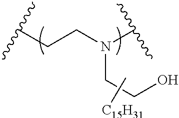 | — |
| 31 | 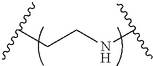 | 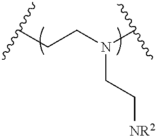 | 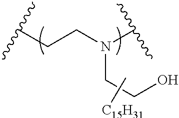 | 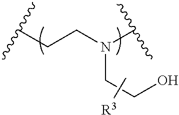 |
| 32 | — | 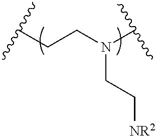 | 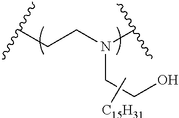 | 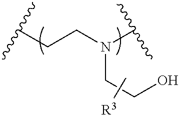 |
| 33 | — | 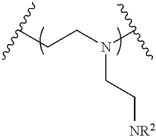 | 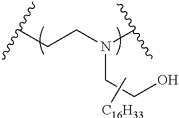 | — |
| 34 | 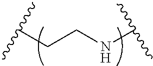 | 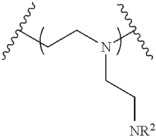 | 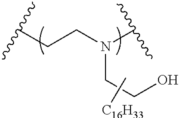 | — |
| 35 | 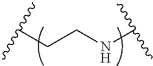 | 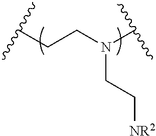 | 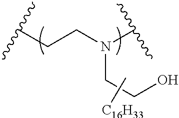 | 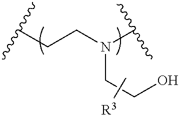 |
| 36 | — | 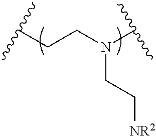 | 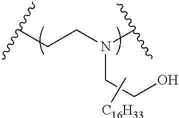 | 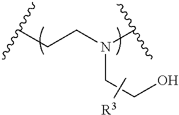 |

In certain embodiments, at least one instance of R⁴ is hydrogen. In certain embodiments, at least one instance of R⁴ is acyl. In certain embodiments, at least one instance of R⁴ is silyl. In certain embodiments, at least one instance of R⁴ is a hydroxyl protecting group. In certain embodiments, at least one instance of R⁴ is substituted or unsubstituted alkyl. In certain embodiments, at least one instance of R⁴ is substituted or unsubstituted alkenyl. In certain embodiments, at least one instance of R⁴ is substituted or unsubstituted alkynyl. In certain embodiments, at least one instance of R⁴ is substituted or unsubstituted heteroalkyl. In certain embodiments, at least one instance of R⁴ is substituted or unsubstituted heteroalkenyl. In certain embodiments, at least one instance of R⁴ is substituted or unsubstituted heteroalkynyl. In certain embodiments, at least one instance of R⁴ is substituted or unsubstituted carbocyclyl. In certain embodiments, at least one instance of R⁴ is substituted or unsubstituted heterocyclyl. In certain embodiments, at least one instance of R⁴ is substituted or unsubstituted aryl. In certain embodiments, at least one instance of R⁴ is substituted or unsubstituted heteroaryl.

In certain embodiments, A is —N(R⁵)₂, wherein at least one instance of R⁵ is hydrogen. In certain embodiments, A is —N(R⁵)₂, wherein each R⁵ is hydrogen. In certain embodiments, A is —N(H)₂. In certain embodiments, A is —N(R⁵)₂, wherein each instance of R⁵ is independently hydrogen; acyl; silyl; sulfonyl; an amino protecting group; substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; substituted or unsubstituted carbocyclyl; substituted or unsubstituted heterocyclyl; substituted or unsubstituted aryl; substituted or unsubstituted heteroaryl; or a group of the formula (iii′); or two R⁵ groups are joined to form a substituted or unsubstituted heterocyclyl. In certain embodiments, at least one instance of R⁵ is hydrogen. In certain embodiments, at least one instance of R⁵ is acyl. In certain embodiments, at least one instance of R⁵ is silyl. In certain embodiments, at least one instance of R⁵ is sulfonyl; an amino protecting group. In certain embodiments, at least one instance of R⁵ is substituted or unsubstituted alkyl. In certain embodiments, at least one instance of R⁵ is substituted or unsubstituted alkenyl. In certain embodiments, at least one instance of R⁵ is substituted or unsubstituted alkynyl. In certain embodiments, at least one instance of R⁵ is substituted or unsubstituted heteroalkyl. In certain embodiments, at least one instance of R⁵ is substituted or unsubstituted heteroalkenyl. In certain embodiments, at least one instance of R⁵ is substituted or unsubstituted heteroalkynyl. In certain embodiments, at least one instance of R⁵ is substituted or unsubstituted carbocyclyl. In certain embodiments, at least one instance of R⁵ is substituted or unsubstituted heterocyclyl. In certain embodiments, at least one instance of R⁵ is substituted or unsubstituted aryl. In certain embodiments, at least one instance of R⁵ is substituted or unsubstituted heteroaryl. In certain embodiments, at least one instance of R⁵ is of the formula (iii′):

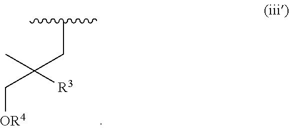

In certain embodiments, two R⁵ groups are joined to form a substituted or unsubstituted heterocyclyl.

In certain embodiments, Z is hydrogen. In certain embodiments, Z is acyl. In certain embodiments, Z is silyl. In certain embodiments, Z is sulfonyl. In certain embodiments, Z is an amino protecting group. In certain embodiments, Z is substituted or unsubstituted alkyl. In certain embodiments, Z is substituted or unsubstituted C₁₋₆ alkyl (e.g., substituted or unsubstituted methyl, ethyl, or propyl). In certain embodiments, Z is substituted or unsubstituted methyl. In certain embodiments, Z is methyl. In certain embodiments, Z is

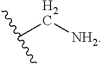 [SMILES: *CN]

In certain embodiments, Z is substituted or unsubstituted alkenyl. In certain embodiments, Z is substituted or unsubstituted alkynyl. In certain embodiments, Z is substituted or unsubstituted heteroalkyl. In certain embodiments, Z is substituted or unsubstituted heteroalkenyl. In certain embodiments, Z is substituted or unsubstituted heteroalkynyl. In certain embodiments, Z is substituted or unsubstituted carbocyclyl. In certain embodiments, Z is substituted or unsubstituted heterocyclyl. In certain embodiments, Z is substituted or unsubstituted aryl. In certain embodiments, Z is substituted or unsubstituted heteroaryl. In certain embodiments, Z is a group of the formula (iii′):

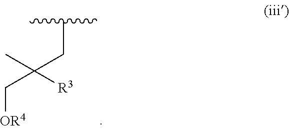

In certain embodiments, Z and the nitrogen atom to which it is attached form a substituted or unsubstituted heterocyclyl group.

In certain embodiments, the conjugated lipomer is of the formula:

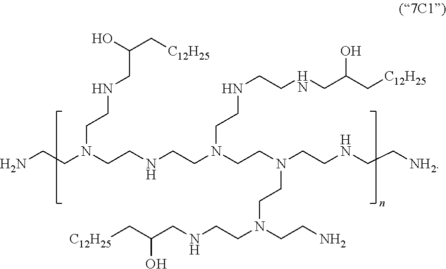

In certain embodiments, the conjugated lipomer is not of formula (“7C1”).

### Lipid-PEG Conjugate

In certain embodiments, the lipid-polyethylene glycol (PEG) conjugate is of Formula (II):

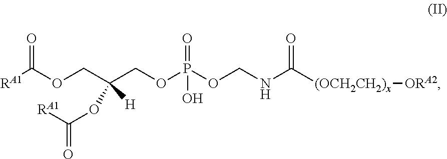

or a pharmaceutically acceptable salt thereof,
wherein:

each instance of R^{A1} is independently unsubstituted C₆-C₂₀ alkyl;

R^{A2} is substituted or unsubstituted alkyl; and

x is an integer between 15 to 135, inclusive.

In certain embodiments, the polyethylene glycol (PEG) of the lipid-PEG conjugate has a molecular weight of approximately 600 g/mol to 750 g/mol. In certain embodiments, the PEG of the lipid-PEG conjugate of Formula (II) has a molecular weight between approximately 750 g/mol and approximately 5000 g/mol. In certain embodiments, the PEG of the lipid-PEG conjugate of Formula (II) has a molecular weight between approximately 750 g/mol and approximately 4500 g/mol. In certain embodiments, the PEG of the lipid-PEG conjugate of Formula (II) has a molecular weight between approximately 750 g/mol and approximately 4000 g/mol. In certain embodiments, the PEG of the lipid-PEG conjugate of Formula (II) has a molecular weight that is not between approximately 2000 g/mol and approximately 5000 g/mol. In certain embodiments, the PEG of the lipid-PEG conjugate of Formula (II) has a molecular weight that is not between approximately 2000 g/mol and approximately 3000 g/mol. In certain embodiments, the PEG of the lipid-PEG conjugate of Formula (II) has a molecular weight that is not between approximately 2000 g/mol and approximately 4000 g/mol. In certain embodiments, the PEG of the lipid-PEG conjugate of Formula (II) has a molecular weight that is not between approximately 3000 g/mol and approximately 5000 g/mol. In certain embodiments, the PEG of the lipid-PEG conjugate of Formula (II) has a molecular weight that is not between approximately 4000 g/mol and approximately 5000 g/mol. In certain embodiments, the PEG of the lipid-PEG conjugate of Formula (II) has a molecular weight between approximately 750 g/mol and approximately 3500 g/mol. In certain embodiments, the PEG of the lipid-PEG conjugate of Formula (II) has a molecular weight between approximately 750 g/mol and approximately 3000 g/mol. In certain embodiments, the PEG of the lipid-PEG conjugate of Formula (II) has a molecular weight between approximately 750 g/mol and approximately 2500 g/mol. In certain embodiments, the PEG of the lipid-PEG conjugate of Formula (II) has a molecular weight between approximately 750 g/mol and approximately 2000 g/mol. In certain embodiments, the PEG of the lipid-PEG conjugate of Formula (II) has a molecular weight between approximately 750 g/mol and approximately 1500 g/mol. In certain embodiments, the PEG of the lipid-PEG conjugate of Formula (II) has a molecular weight between approximately 750 g/mol and approximately 1000 g/mol. In certain embodiments, the PEG of the lipid-PEG conjugate of Formula (II) has a molecular weight between approximately 900 g/mol to 1100 g/mol. In certain embodiments, the PEG of the lipid-PEG conjugate of Formula (II) has a molecular weight between approximately 1000 g/mol to 2500 g/mol. In certain embodiments, the PEG of the lipid-PEG conjugate of Formula (II) has a molecular weight between approximately 1500 g/mol to 2450 g/mol. In certain embodiments, the PEG of the lipid-PEG conjugate of Formula (II) has a molecular weight between approximately 2000 g/mol to 3000 g/mol. In certain embodiments, the PEG of the lipid-PEG conjugate of Formula (II) has a molecular weight between approximately 2500 g/mol to 3000 g/mol. In certain embodiments, the PEG of the lipid-PEG conjugate of Formula (II) has a molecular weight between approximately 4000 g/mol to 5500 g/mol. In certain embodiments, the PEG of the lipid-PEG conjugate of Formula (II) has a molecular weight between approximately 4500 g/mol to 5500 g/mol. In certain embodiments, the PEG of the lipid-PEG conjugate has a molecular weight of approximately 600 g/mol to 750 g/mol, approximately 900 g/mol to 1100 g/mol, approximately 1500 g/mol to 2450 g/mol, approximately 2500 g/mol to 3000 g/mol, or approximately 4500 g/mol to 5500 g/mol.

The lipid-PEG conjugate of Formula (II) includes substituent R^{A1}. In certain embodiments, each instance of R^{A1} is independently unsubstituted C₆-C₂₀ alkyl. In certain embodiments, at least one instance of R^{A1} is unsubstituted C₆-C₁₅ alkyl. In certain embodiments, at least one instance of R^{A1} is unsubstituted C₆-C₁₂ alkyl. In certain embodiments, at least one instance of R^{A1} is unsubstituted C₁₄-C₁₈ alkyl. In certain embodiments, at least one instance of R^{A1} is unsubstituted C₁₃-C₁₈ alkyl. In certain embodiments, at least one instance of R^{A1} is unsubstituted C₁₃ alkyl. In certain embodiments, at least one instance of R^{A1} is unsubstituted C₁₄ alkyl. In certain embodiments, at least one instance of R^{A1} is not unsubstituted C₁₄ alkyl. In certain embodiments, at least one instance of R^{A1} is unsubstituted C₁₅ alkyl. In certain embodiments, at least one instance of R^{A1} is unsubstituted C₁₆ alkyl. In certain embodiments, at least one instance of R^{A1} is not unsubstituted C₁₆ alkyl. In certain embodiments, at least one instance of R^{A1} is unsubstituted C₁₇ alkyl. In certain embodiments, at least one instance of R^{A1} is unsubstituted C₁₈ alkyl. In certain embodiments, at least one instance of R^{A1} is not unsubstituted C₁₈ alkyl. In certain embodiments, at least one instance of R^{A1} is unsubstituted C₁₉ alkyl. In certain embodiments, at least one instance of R^{A1} is unsubstituted C₂₀ alkyl.

The lipid-PEG conjugate of Formula (II) includes substituent R^{A2}. In certain embodiments, R^{A2} is substituted or unsubstituted alkyl. In certain embodiments, R^{A2} is substituted or unsubstituted C₁-C₁₂ alkyl. In certain embodiments, R^{A2} is substituted or unsubstituted C₁-C₆ alkyl. In certain embodiments, R^{A2} is unsubstituted methyl. In certain embodiments, R^{A2} is ethyl. In certain embodiments, R^{A2} is propyl. In certain embodiments, R^{A2} is butyl.

In certain embodiments, the lipid-PEG conjugate of Formula (II) is of the formula:

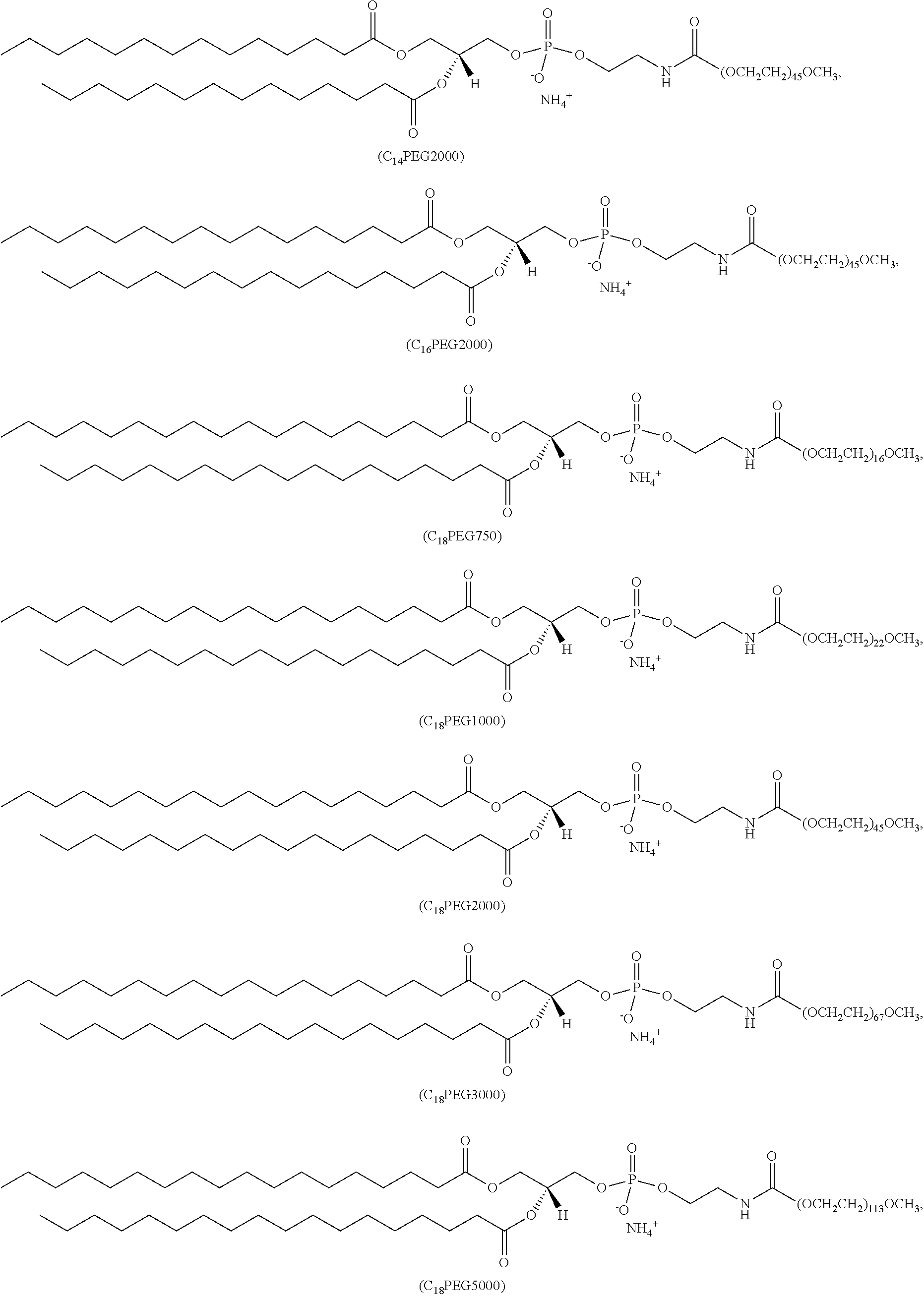

or a pharmaceutically acceptable salt thereof.

As generally defined within, x is an integer between 15 to 135, inclusive. In certain embodiments, x is an integer between 110 to 135, inclusive. In certain embodiments, x is 113. In certain embodiments, x is an integer between 15 to 120, inclusive. In certain embodiments, x is an integer between 20 to 115, inclusive. In certain embodiments, x is an integer between 15 to 115, inclusive. In certain embodiments, x is about 15. In certain embodiments, x is about 16. In certain embodiments, x is an integer between 20 to 100, inclusive. In certain embodiments, x is an integer between 20 to 75, inclusive. In certain embodiments, x is an integer between 20 to 50, inclusive. In certain embodiments, x is about 22. In certain embodiments, x is about 25. In certain embodiments, x is an integer between 20-25. In certain embodiments, x is about 45. In certain embodiments, x is an integer between 25-46. In certain embodiments, x is an integer between 50 to 75, inclusive. In certain embodiments, x is 67. In certain embodiments, x is an integer between 55 to 70, inclusive. In certain embodiments, x is 16, 22, 45, 67, or 113.

In certain embodiments, the molar percentage of the lipid-PEG conjugate in the composition comprising a conjugated lipomer of Formula (I) and the lipid-PEG conjugate of Formula (II) is about 5 molar percent to about 50 molar percent. In certain embodiments, the molar percentage of the lipid-PEG conjugate in the composition is about 4 molar percent to about 50 molar percent. In certain embodiments, the molar percentage of the lipid-PEG conjugate in the composition is about 4 molar percent to about 32 molar percent. In certain embodiments, the molar percentage of the conjugated lipomer in the composition is about 4 molar percent, about 5 molar percent, about 8 molar percent, about 10 molar percent, about 12 molar percent, about 15 molar percent, about 16 molar percent, about 18 molar percent, about 20 molar percent, about 24 molar percent, about 25 molar percent, about 28 molar percent, about 30 molar percent, about 32 molar percent, about 35 molar percent, about 38 molar percent, about 40 molar percent, about 45 molar percent, or about 50 molar percent.

In certain embodiments, the molar percentage of the conjugated lipomer in the composition is about 4 molar percent. In certain embodiments, the molar percentage of the conjugated lipomer in the composition is about 8 molar percent. In certain embodiments, the molar percentage of the conjugated lipomer in the composition is about 10 molar percent. In certain embodiments, the molar percentage of the conjugated lipomer in the composition is about 16 molar percent. In certain embodiments, the molar percentage of the conjugated lipomer in the composition is about 24 molar percent. In certain embodiments, the molar percentage of the conjugated lipomer in the composition is about 32 molar percent.

In certain embodiments, the molar percentage of the conjugated lipomer in the composition comprising a conjugated lipomer of Formula (I) and the lipid-PEG conjugate of Formula (II) is about 60 molar percent to about 96 molar percent. In certain embodiments, the molar percentage of the conjugated lipomer in the composition is about 68 molar percent to about 96 molar percent. In certain embodiments, the molar percentage of the conjugated lipomer in the composition is about 60 molar percent, about 62 molar percent, about 65 molar percent, about 68 molar percent, about 70 molar percent, about 72 molar percent, about 75 molar percent, about 76 molar percent, about 80 molar percent, about 82 molar percent, about 84 molar percent, about 85 molar percent, about 90 molar percent, about 92 molar percent, or about 96 molar percent. In certain embodiments, the molar percentage of the conjugated lipomer in the composition is about 68 molar percent. In certain embodiments, the molar percentage of the conjugated lipomer in the composition is about 76 molar percent. In certain embodiments, the molar percentage of the conjugated lipomer in the composition is about 84 molar percent. In certain embodiments, the molar percentage of the conjugated lipomer in the composition is about 92 molar percent. In certain embodiments, the molar percentage of the conjugated lipomer in the composition is about 96 molar percent.

In certain embodiments, the composition includes a particle, wherein the particle comprises a conjugated lipomer of Formula (I); and a lipid-polyethylene glycol (PEG) conjugate of Formula (II). In certain embodiments, the particle is a nanoparticle. In certain embodiments, the composition includes a particle. In certain embodiments, the composition comprises 84 molar percent of conjugated lipomer 7C1 and 16 molar percent of C₁₄PEG2000, wherein the composition is used to synthesize a particle. In certain embodiments, the composition comprises 84 molar percent of conjugated lipomer 7C1 and 16 molar percent of C₁₆PEG2000, wherein the composition is used to synthesize a particle. In certain embodiments, the composition comprises 84 molar percent of conjugated lipomer 7C1 and 16 molar percent of C₁₈PEG750, wherein the composition is used to synthesize a particle. In certain embodiments, the composition comprises 84 molar percent of conjugated lipomer 7C1 and 16 molar percent of C₁₈PEG1000, wherein the composition is used to synthesize a particle. In certain embodiments, the composition comprises 84 molar percent of conjugated lipomer 7C1 and 16 molar percent of C₁₈PEG2000, wherein the composition is used to synthesize a particle. In certain embodiments, the composition comprises 84 molar percent of conjugated lipomer 7C1 and 16 molar percent of C₁₈PEG3000, wherein the composition is used to synthesize a particle. In certain embodiments, the composition comprises 84 molar percent of conjugated lipomer 7C1 and 16 molar percent of C₁₈PEG5000, wherein the composition is used to synthesize a particle. In certain embodiments, the composition comprises 68 molar percent of conjugated lipomer 7C1 and 32 molar percent of C₁₈PEG5000, wherein the composition is used to synthesize a particle. In certain embodiments, the composition comprises 76 molar percent of conjugated lipomer 7C1 and 24 molar percent of C₁₈PEG5000, wherein the composition is used to synthesize a particle. In certain embodiments, the composition comprises 92 molar percent of conjugated lipomer 7C1 and 8 molar percent of C₁₈PEG5000, wherein the composition is used to synthesize a particle. In certain embodiments, the composition comprises 96 molar percent of conjugated lipomer 7C1 and 4 molar percent of C₁₈PEG5000, wherein the composition is used to synthesize a particle. In certain embodiments, the composition does not comprise 2 molar percent of PEG. In certain embodiments, the composition does not comprise 5 molar percent of PEG. In certain embodiments, the composition does not comprise 10 molar percent of PEG. In certain embodiments, the composition does not comprise 15 molar percent of PEG. In certain embodiments, the composition does not comprise 20 molar percent of PEG.

### Particles

The composition comprising a conjugated lipomer of Formula (I); and a lipid-polyethylene glycol (PEG) conjugate of Formula (II) of the present invention may also be used to form drug delivery devices (e.g., particles). The inventive composition comprising a conjugated lipomer of Formula (I); and a lipid-polyethylene glycol (PEG) conjugate of Formula (II) have several properties that make them particularly suitable in the preparation of drug delivery devices. These include: 1) the ability of the lipomer to complex and “protect” labile agents; 2) the ability to buffer the pH in the endosome; 3) the ability to act as a “proton sponge” and cause endosomolysis; and 4) the ability to neutralize the charge on negatively charged agents. The present invention provides particles comprising both: conjugated lipomer of Formula (I); and a lipid-polyethylene glycol (PEG) conjugate of Formula (II). In certain embodiments, the particle is a nanoparticle.

In certain embodiments, the composition comprising a conjugated lipomer of Formula (I); and a lipid-polyethylene glycol (PEG) conjugate of Formula (II) are used to form particles containing the agent to be delivered. The inventive composition comprising a conjugated lipomer of Formula (I) and a lipid-polyethylene glycol (PEG) conjugate of Formula (II) may be used to encapsulate agents including, but not limited to, organic molecules, inorganic molecules, nucleic acids, proteins, peptides, polynucleotides, targeting agents, isotopically labeled organic or inorganic molecules, vaccines, immunological agents, etc. Other exemplary agents are described in greater detail herein. These particles may include other materials, such as polymers (e.g., synthetic polymers (e.g., PEG, PLGA), natural polymers (e.g., proteins, phospholipids)).

In certain embodiments, the diameter of the particles range from between 1 micrometer to 1,000 micrometers. In certain embodiments, the diameter of the particles range from between from 1 micrometer to 100 micrometers. In certain embodiments, the diameter of the particles range from between from 1 micrometer to 10 micrometers. In certain embodiments, the diameter of the particles range from between from 10 micrometer to 100 micrometers. In certain embodiments, the diameter of the particles range from between from 100 micrometer to 1,000 micrometers. In certain embodiments, the particles range from 1-5 micrometers. In certain embodiments, the diameter of the particles range from between 1 nm to 1,000 nm. In certain embodiments, the diameter of the particles range from between from 1 nm to 100 nm. In certain embodiments, the diameter of the particles range from between from 1 nm to 10 nm. In certain embodiments, the diameter of the particles range from between from 10 nm to 100 nm. In certain embodiments, the diameter of the particles range from between from 100 nm to 1,000 nm. In certain embodiments, the particles range from 1-5 nm. In certain embodiments, the diameter of the particles range from between 1 pm to 1,000 pm. In certain embodiments, the diameter of the particles range from between from 1 pm to 100 pm. In certain embodiments, the diameter of the particles range from between from 1 pm to 10 pm. In certain embodiments, the diameter of the particles range from between from 10 pm to 100 pm. In certain embodiments, the diameter of the particles range from between from 100 pm to 1,000 pm. In certain embodiments, the particles range from 1-5 pm.

The inventive particles may be prepared using any method known in this art. These include, but are not limited to, spray drying, single and double emulsion solvent evaporation, solvent extraction, phase separation, simple and complex coacervation, and other methods well known to those of ordinary skill in the art. In certain embodiments, methods of preparing the particles are the double emulsion process and spray drying. The conditions used in preparing the particles may be altered to yield particles of a desired size or property (e.g., hydrophobicity, hydrophilicity, external morphology, “stickiness”, shape, etc.). The method of preparing the particle and the conditions (e.g., solvent, temperature, concentration, air flow rate, etc.) used may also depend on the agent being encapsulated and/or the composition of the matrix.

Methods developed for making particles for delivery of encapsulated agents are described in the literature (for example, please see Doubrow, M., Ed., “Microcapsules and Nanoparticles in Medicine and Pharmacy,” CRC Press, Boca Raton, 1992; Mathiowitz and Langer, J. Controlled Release 5:13-22, 1987; Mathiowitz et al., Reactive Polymers 6:275-283, 1987; Mathiowitz et al., J. Appl. Polymer Sci. 35:755-774, 1988; each of which is incorporated herein by reference).

If the particles prepared by any of the above methods have a size range outside of the desired range, the particles can be sized, for example, using a sieve. The particle may also be coated. In certain embodiments, the particles are coated with a targeting agent. In other embodiments, the particles are coated to achieve desirable surface properties (e.g., a particular charge).

### Agents

The agents to be delivered by the systems of the present invention may be therapeutic, diagnostic, or prophylactic agents. Any chemical compound to be administered to an individual may be delivered using the inventive complexes or particles. The agent may be an organic molecule (e.g., a drug), inorganic molecule, nucleic acid, protein, peptide, polynucleotide, targeting agent, isotopically labeled organic or inorganic molecule, vaccine, immunological agent, etc.

In certain embodiments, the agents are organic molecules with pharmaceutical activity, e.g., a drug. In certain embodiments, the drug is an antibiotic, anti-viral agent, anesthetic, steroidal agent, anti-inflammatory agent, anti-neoplastic agent, anti-cancer agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, non-steroidal anti-inflammatory agent, nutritional agent, etc. For example, in certain embodiments, the agent is a small molecule, organometallic compound, nucleic acid, protein, peptide, polynucleotide, metal, targeting agent, an isotopically labeled chemical compound, drug, vaccine, or immunological agent.

In certain embodiments, the agent is a polynucleotide. In certain embodiments, the polynucleotide carries out RNA interference In certain embodiments, the polynucleotide or nucleic acid is a double- or single-stranded genomic and cDNA, RNA, any synthetic and genetically manipulated polynucleotide, or both sense and antisense polynucleotides.

In certain embodiments, the nucleic acid is a single- or double-stranded molecule, i.e., DNA-DNA, DNA-RNA or RNA-RNA hybrids, or “protein nucleic acids” (PNAs) formed by conjugating bases to an amino acid backbone. In certain embodiments, the nucleic acid is a nucleic acid containing carbohydrates or lipids. In certain embodiments, the polynucleotide is DNA or RNA. In certain embodiments, the DNA is single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, or viral DNA. In certain embodiments, the RNA is single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, or viral satellite RNA. In certain embodiments, the RNA is RNAi, dsRNA, siRNA, shRNA, miRNA, or antisense RNA.

In certain particular embodiments, the agent is siRNA. An siRNA molecule used in a composition (e.g., nanoparticle) of the invention is a duplex consisting of a sense strand and complementary antisense strand, the antisense strand having sufficient complementary to a a target mRNA to mediate RNAi. Preferably, the siRNA molecule has a length from about 10-50 or more nucleotides, i.e., each strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in each strand, wherein the antisense strand is sufficiently complementary to a target region. Preferably, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. siRNA may comprise chemically modified nucleotides (e.g., the 2′ OH-group may be replaced by a group selected from H, OR, R, F, Cl, Br, I, SH, SR, NH₂, NHR, NR₂, COOR, or OR, wherein R is substituted or unsubstituted C₁-C₆ alkyl) or internucleotide linkages (e.g., phosphorothioate linkages).

Generally, siRNAs can be designed by using any method known in the art. siRNA targeting a particular mRNA transcript will comprise a sequence that is homologous to a section (e.g., 5-50 nucleotides) of DNA. Representative Genbank accession numbers providing DNA and mRNA sequence information are: SDF-1: NC_000072 REGION: 117168535..117181368 (NCBI Reference Sequence: NC_000072.6); MCP-1: NC_000077 REGION: 82035577..82037452 (NCBI Reference Sequence: NC_000077.6); Tie2: NC_000070 REGION: 94739289..94874976 (NCBI Reference Sequence: NC_000070.6), and are incorporated herein by reference.

In certain particular embodiments, the siRNA targets Sdf-1, Mcp-1, or Tie2.

In a particular embodiment, the siRNA targets Tie2 and comprises a sense strand having the sequence: GAAGAuGcAGuGAuuuAcAdTsdT (SEQ ID NO: 1); and an antisense strand having the sequence: UGuAAAUcACUGcAUCUUCdTsdT (SEQ ID NO: 2) (lower caes letters correspond to 2′-OCH₃ modified nucleotides).

In certain embodiments, the siRNA targets a protein of a hematopoietic stem and progenitor cells (HSPC). In certain embodiments, the siRNA targets a protein of a bone marrow endothelial cells (BMEC).

In certain embodiments of the present invention, the agent to be delivered may be a mixture of agents.

Diagnostic agents include gases; metals; commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium. Examples of materials useful for CAT and x-ray imaging include iodine-based materials.

Prophylactic agents include, but are not limited to, antibiotics, nutritional supplements, and vaccines. Vaccines may comprise isolated proteins or peptides, inactivated organisms and viruses, dead organisms and viruses, genetically altered organisms or viruses, and cell extracts. Prophylactic agents may be combined with interleukins, interferon, cytokines, and adjuvants such as cholera toxin, alum, Freund's adjuvant, etc. Prophylactic agents include antigens of such bacterial organisms as Streptococccus pneumoniae, Haemophilus influenzae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, Camphylobacter jejuni, and the like; antigens of such viruses as smallpox, influenza A and B, respiratory syncytial virus, parainfluenza, measles, HIV, varicella-zoster, herpes simplex 1 and 2, cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, and the like; antigens of fungal, protozoan, and parasitic organisms such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

### Targeting Agents

The conjugated lipomer of Formula (I); and a lipid-polyethylene glycol (PEG) conjugate of Formula (II) and particles, may be modified to include targeting agents since it is often desirable to target a particular cell, collection of cells, or tissue. A variety of targeting agents that direct pharmaceutical compositions to particular cells are known in the art (see, for example, Cotten et al., Methods Enzym. 217:618, 1993; incorporated herein by reference). The targeting agents may be included throughout the particle or may be only on the surface. The targeting agent may be a protein, peptide, carbohydrate, glycoprotein, lipid, small molecule, nucleic acids, etc. The targeting agent may be used to target specific cells or tissues or may be used to promote endocytosis or phagocytosis of the particle. Examples of targeting agents include, but are not limited to, antibodies, fragments of antibodies, low-density lipoproteins (LDLs), transferrin, asialycoproteins, gp120 envelope protein of the human immunodeficiency virus (HIV), carbohydrates, receptor ligands, sialic acid, aptamers, etc. If the targeting agent is included throughout the particle, the targeting agent may be included in the mixture that is used to form the particles. If the targeting agent is only on the surface, the targeting agent may be associated with (i.e., by covalent, hydrophobic, hydrogen bonding, van der Waals, or other interactions) the formed particles using standard chemical techniques.

### Pharmaceutical Compositions

The present invention contemplates a composition comprising a particle (e.g., a nanoparticle) comprising a conjugated lipomer and a lipid-PEG conjugate, which may be useful in a variety of medical and non-medical applications. For example, pharmaceutical compositions comprising a conjugated lipomer and a lipid-PEG conjugate as components may be useful in the delivery of an effective amount of an agent to a subject in need thereof. Nutraceutical compositions comprising a conjugated lipomer and a lipid-PEG conjugate as components may be useful in the delivery of an effective amount of a nutraceutical, e.g., a dietary supplement, to a subject in need thereof. Cosmetic compositions comprising a conjugated lipomer and a lipid-PEG conjugate as components may be formulated as a cream, ointment, balm, paste, film, or liquid, etc., and may be useful in the application of make-up, hair products, and materials useful for personal hygiene, etc. Compositions comprising a conjugated lipomer and a lipid-PEG conjugate as components may be useful for non-medical applications, e.g., such as an emulsion or emulsifier, useful, for example, as a food component, for extinguishing fires, for disinfecting surfaces, for oil cleanup, etc.

In certain embodiments, the composition comprises one or more conjugated lipomers. “One or more conjugated lipomers” refers to one or more different types of conjugated lipomers included in the composition, and encompasses 1, 2, 3, 4, 5, 6, 7, 8, 9, 10 or more different types of conjugated lipomers.

In certain embodiments, the composition comprises a lipid-PEG conjugate of Formula (II):

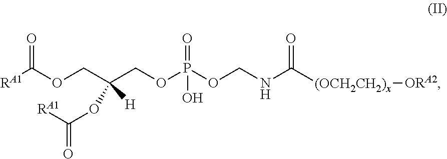

or a pharmaceutically acceptable salt thereof.

In certain embodiments, a conjugated lipomer and a lipid-PEG conjugate as components of a composition are useful either for delivery of an effective amount of an agent to a subject in need thereof (e.g., a pharmaceutical composition, a cosmetic composition) or for use as an excipient. For example, cosmetic compositions may further use the conjugated lipomer and a lipid-PEG conjugate as excipients rather than as a delivery system encapsulating an agent to be delivered. In certain embodiments, the composition is a pharmaceutical composition. In certain embodiments, the composition is a cosmetic composition.

In certain embodiments, the composition further comprises an agent, as described herein. For example, in certain embodiments, the agent is a small molecule, organometallic compound, nucleic acid, protein, peptide, polynucleotide, metal, targeting agent, an isotopically labeled chemical compound, drug, vaccine, or immunological agent. In certain embodiments, the agent is a polynucleotide.

In certain embodiments, the polynucleotide and the one or more conjugated lipomers and/or lipid-PEG conjugate are not covalently attached.

In certain embodiments, the one or more conjugated lipomers and lipid-PEG conjugate are in the form of a particle. In certain embodiments, the particle is a nanoparticle or microparticle. In certain embodiments, the particle encapsulates an agent. The agent to be delivered by the particles may be in the form of a gas, liquid, or solid. The conjugated lipomers may be combined with polymers (synthetic or natural), surfactants, cholesterol, carbohydrates, proteins, lipids etc. to form the particles. These particles may be combined with an excipient to form pharmaceutical and cosmetic compositions.

Once the complexes, liposomes, or particles have been prepared, they may be combined with one or more excipients to form a composition that is suitable to administer to animals including humans.

As would be appreciated by one of skill in this art, the excipients may be chosen based on the route of administration as described below, the agent being delivered, time course of delivery of the agent, etc.

In certain embodiments, provided is a composition comprising a conjugated lipomer, a lipid-PEG conjugate and, optionally, an excipient. As used herein, the term “excipient” means a non-toxic, inert solid, semi-solid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The compositions of this invention can be administered to humans and/or to animals, orally, rectally, parenterally, intracisternally, intravaginally, intranasally, intraperitoneally, topically (as by powders, creams, ointments, or drops), bucally, or as an oral or nasal spray.

Liquid dosage forms for oral administration include emulsions, microemulsions, solutions, suspensions, syrups, and elixirs. In addition to the active ingredients (i.e., microparticles, nanoparticles, liposomes, micelles, polynucleotide/lipid complexes), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid dosage forms for oral administration include capsules, tablets, pills, powders, and granules. In such solid dosage forms, the particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Solid compositions of a similar type may also be employed as fillers in soft and hard-filled gelatin capsules using such excipients as lactose or milk sugar as well as high molecular weight polyethylene glycols and the like.

Dosage forms for topical or transdermal administration of an inventive pharmaceutical composition include ointments, pastes, creams, lotions, gels, powders, solutions, sprays, inhalants, or patches. The particles are admixed under sterile conditions with a pharmaceutically acceptable carrier and any needed preservatives or buffers as may be required. Ophthalmic formulation, ear drops, and eye drops are also contemplated as being within the scope of this invention.

The ointments, pastes, creams, and gels may contain, in addition to the particles of this invention, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the particles of this invention, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the microparticles or nanoparticles in a proper medium. Absorption enhancers can also be used to increase the flux of the compound across the skin. The rate can be controlled by either providing a rate controlling membrane or by dispersing the particles in a polymer matrix or gel.

Also encompassed by the disclosure are kits (e.g., pharmaceutical packs). The kits provided may comprise a composition or particle described herein and a container (e.g., a vial, ampule, bottle, syringe, and/or dispenser package, or other suitable container). In some embodiments, provided kits may optionally further include a second container comprising a pharmaceutical excipient for dilution or suspension of a composition or particle described herein. In some embodiments, the composition or particle described herein provided in the first container and the second container are combined to form one unit dosage form.

Thus, in one aspect, provided are kits including a first container comprising a composition or particle described herein. In certain embodiments, the kits are useful for treating a disease (e.g., proliferative disease, inflammatory disease, autoinflammatory disease, autoimmune disease, genetic disease (e.g., bone marrow disease), hematological disease, immunological disorder, and/or hematological disorder) in a subject in need thereof. In certain embodiments, the kits are useful for preventing a disease (e.g., proliferative disease, inflammatory disease, autoinflammatory disease, autoimmune disease, genetic disease (e.g., bone marrow disease), hematological disease, immunological disorder, and/or hematological disorder) in a subject in need thereof.

In certain embodiments, a kit described herein further includes instructions for using the particle or composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease, inflammatory disease, autoinflammatory disease, autoimmune disease, genetic disease (e.g., bone marrow disease), hematological disease, immunological disorder, and/or hematological disorder) in a subject in need thereof. In certain embodiments, the kits and instructions provide for preventing a disease (e.g., proliferative disease, inflammatory disease, autoinflammatory disease, autoimmune disease, genetic disease (e.g., bone marrow disease), hematological disease, immunological disorder, and/or hematological disorder) in a subject in need thereof. A kit described herein may include one or more additional pharmaceutical agents described herein as a separate composition.

### Methods of Treatment and Uses

In one aspect, provided are methods for delivering an agent to a cell, comprising contacting the cell with a composition as described herein, e.g., a composition comprising a conjugated lipomer, a lipid-PEG conjugate, and an agent. In a particular embodiment, the composition is a nanoparticle. In a particular embodiment, the composition is NicheEC-15. In a particular embodiment, the agent is siRNA. In a particular embodiment, the cell is a hematopoietic stem and progenitor cell (HSPC) or a bone marrow endothelial cells (BMEC).

In another aspect, provided are methods for delivering an agent to a subject, comprising administereing to the subject a composition as described herein, e.g., a composition comprising a conjugated lipomer, a lipid-PEG conjugate, and an agent. In a particular embodiment, the composition is a nanoparticle. In a particular embodiment, the composition is a pharmaceutical composition comprising a nanoparticle. In a particular embodiment, the pharmaceutical composition comprises NicheEC-15. In a particular embodiment, the agent is siRNA.

In another aspect, provided are methods of using a composition comprising a particle (e.g., a nanoparticle), wherein the particle comprises a conjugated lipomer and a lipid-PEG conjugate, e.g., for the treatment of a disease, disorder or condition from which a subject suffers. It is contemplated that the conjugated lipomer and lipid-PEG conjugate composition will be useful in the treatment of a variety of diseases, disorders or conditions, especially as a system for delivering agents useful in the treatment of that particular disease, disorder or condition (e.g., delivery of a biologically active agent (e.g., nucleic acids) to bone marrow for the treatment of cancer (e.g., bone marrow cancer)) or a bone marrow disease (e.g., genetic disease). In certain embodiments, the biologically active agent is delivered to the subject in vivo. In certain embodiments, the biologically active agent is delivered to bone marrow in vivo. In certain embodiments, the biologically active agent is delivered to bone marrow cells of the subject. In certain embodiments, the biologically active agent is targeted to bone marrow cells of the subject.

For example, in one aspect, provided is a method of treating a disease comprising administering to a subject in need thereof an effective amount of a composition comprising a conjugated lipomer and a lipid-PEG conjugate, e.g., a composition comprising a conjugated lipomer of the Formula (I), or a pharmaceutically acceptable salt thereof, and a lipid- PEG conjugate of Formula (II). In certain embodiments, the method further comprises administering a biologically active agent (e.g. an anti-cancer agent or agent for treating bone marrow disease). In certain embodiments, the composition comprising a conjugated lipomer and a lipid-PEG conjugate encapsulates the biologically active agent (e.g. the anti-cancer agent or agent for treating bone marrow disease). In certain embodiments, the composition comprising a conjugated lipomer and a lipid-PEG conjugate and the biologically active agent form a particle (e.g., a nanoparticle, a microparticle, a micelle, a liposome, a lipoplex).

In another aspect, provided are methods of treating a disease in a subject in need thereof, the method comprising administering to the subject a therapeutically effective amount of a composition as described herein, e.g., a composition comprising a conjugated lipomer, a lipid-PEG conjugate, and an agent. In a particular embodiment, the subject is a mammal. In another particular embodiment, the subject is a human. In certain embodiments, the disease is selected from the group consisting of cardiovascular disease, lung disease, proliferative disease, inflammatory disorders, and immunological disorders.

Exemplary cancers include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenström's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

Anti-cancer agents encompass biotherapeutic anti-cancer agents as well as chemotherapeutic agents.

Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)).

Exemplary chemotherapeutic agents include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genentech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin- aminopterin, and hexamethyl melamine.

In another aspect, provided are methods for delivering a biologically active agent to a subject comprising: administering a composition comprising the biologically active agent to the subject, wherein the composition includes a particle, wherein the particle comprises: a biologically active agent; a conjugated lipomer of Formula (I), or a pharmaceutically acceptable salt thereof, described herein and a lipid- PEG conjugate of Formula (II), or a pharmaceutically acceptable salt thereof, described herein. In certain embodiments, the biologically active agent is delivered to the subject in vivo. In certain embodiments, the biologically active agent is delivered to bone marrow in vivo. In certain embodiments, the biologically active agent is delivered to bone marrow cells in vivo. In certain embodiments, the biologically active agent is targeted to bone marrow cells in vivo.

In certain embodiments, the composition comprising a conjugated lipomer of Formula (I); and a lipid-polyethylene glycol (PEG) conjugate of Formula (II) are mixed with one or more agents and/or one or more other materials (e.g., polymers). In certain embodiments, the composition comprising a conjugated lipomer of Formula (I) and a lipid-polyethylene glycol (PEG) conjugate of Formula (II) is mixed with an agent, to provide inventive particles.

In certain embodiments, the biologically active agent is formulated in a composition with the conjugated lipomer of Formula (I) and lipid-polyethylene glycol (PEG) conjugate of Formula (II) by encapsulating the biologically active agent via microfluidic mixing. In certain embodiments, the microfluidic mixing is conducted in a microfluidic device. In certain embodiments, the biologically active agent is a therapeutic agent. In certain embodiments, the biologically active agent is a diagnostic agent. In certain embodiments, the biologically active agent is a small molecule. In certain embodiments, the biologically active agent is an organometallic compound. In certain embodiments, the biologically active agent is a nucleic acid. In certain embodiments, the biologically active agent is a protein. In certain embodiments, the biologically active agent is a peptide. In certain embodiments, the biologically active agent is a polynucleotide, metal, targeting agent, an isotopically labeled chemical compound, drug, vaccine, or immunological agent. In certain embodiments, the biologically active agent is a polynucleotide (e.g., DNA or RNA). In certain embodiments, the biologically active agent is RNA. In certain embodiments, the RNA is RNAi, dsRNA, siRNA, shRNA, miRNA, or antisense RNA. In certain embodiments, the RNA is siRNA. In certain embodiments, the step of administering the biologically active agent to the subject comprises administering the composition intravenously. In certain embodiments, the biologically active agent formulated as the composition is measured. In certain embodiments, the biologically active agent is measured in bone marrow cells. In certain embodiments, the biologically active agent formulated as the composition is measured by fluorescence-activated cell sorting followed by polymerase chain reaction (PCR) analysis of gene silencing. In certain embodiments, the fluorescence-activated cell sorting is fluorescence-activated cell sorting of marrow cell populations. In certain embodiments, the biologically active agent formulated as the composition is measured by a branched DNA assay to measure mRNA expression in target cells of interest.

The present disclosure also provides methods for the treatment of a wide range of diseases, such as proliferative diseases in a subject in need thereof. The present disclosure provides methods for the treatment and/or prevention of a proliferative disease (e.g., cancers (e.g., bone marrow cancer, leukemia, lymphoma, breast cancer metastasis, leukemia, lymphoma, multiple myeloma, prostate cancer metastasis), inflammatory diseases, autoinflammatory diseases, autoimmune diseases, immunological disorders, immunodeficiencies immunotherapy, hematological disorders, hematopoietic stem cell disorders, hematopoietic stem cell transplantation, sickle cell anemia, environmentally-induced diseases (e.g., radiation poisoning), gene therapy, lysosomal storage disorders, metabolic disorders, single gene disorders, or viral diseases. In certain embodiments, the disease is a bone marrow disease. In certain embodiments, the bone marrow disease is anemia (e.g., aplastic anemia). In certain embodiments, the bone marrow disease is a myeloproliferative disorder. In certain embodiments, the disease is a proliferative disease. In certain embodiments, the proliferative disease is cancer. In certain embodiments, the cancer is bone marrow cancer. In certain embodiments, the cancer is leukemia. In certain embodiments, the cancer is lymphoma. In certain embodiments, the disease is an immunological disorder. In certain embodiments, the disease is a hematological disorder.

### EXAMPLES

In order that the invention described herein may be more fully understood, the following examples are set forth. The synthetic and biological examples described in this application are offered to illustrate the compounds, compositions, pharmaceutical compositions, particles, and methods provided herein and are not to be construed in any way as limiting their scope.

### Biological Applications

Patients which have metastasis in bone and bone marrow have poor prognosis. Cancers (e.g., epithelial and blood cancers) that colonize in bone and bone marrow are difficult to treat due to inefficient delivery of drugs to these tissues. The compositions described in this disclosure are used to deliver nucleic acids to the tumor microenvironment. Furthermore, nucleic acids can now be delivered using these compositions silence “undruggable” targets within tumor cells within these tissues, enabling these tumors to now respond to chemo-, radio-, and immunotherapies. Applicable cancers for these compositions include: metastatic breast and prostate cancer, multiple myeloma, leukemia, and lymphoma.

### Example 1

### Nanoparticle Screen for In Vivo siRNA Delivery to the Hematopoietic Niche

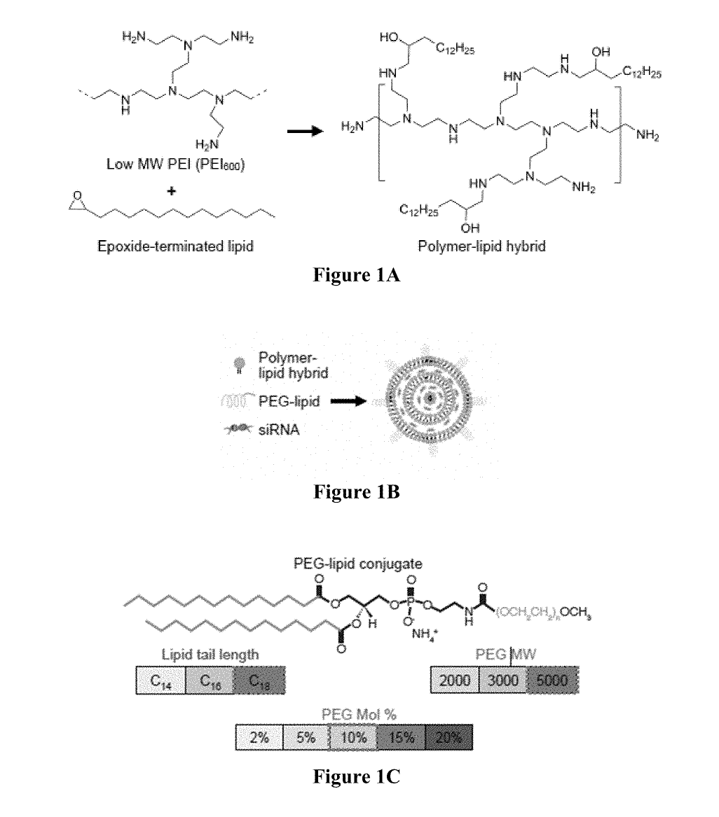

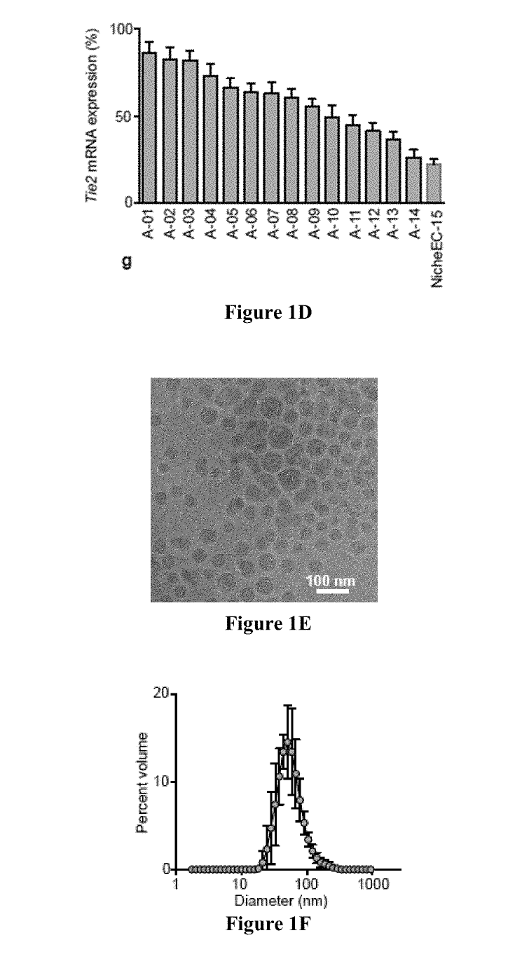

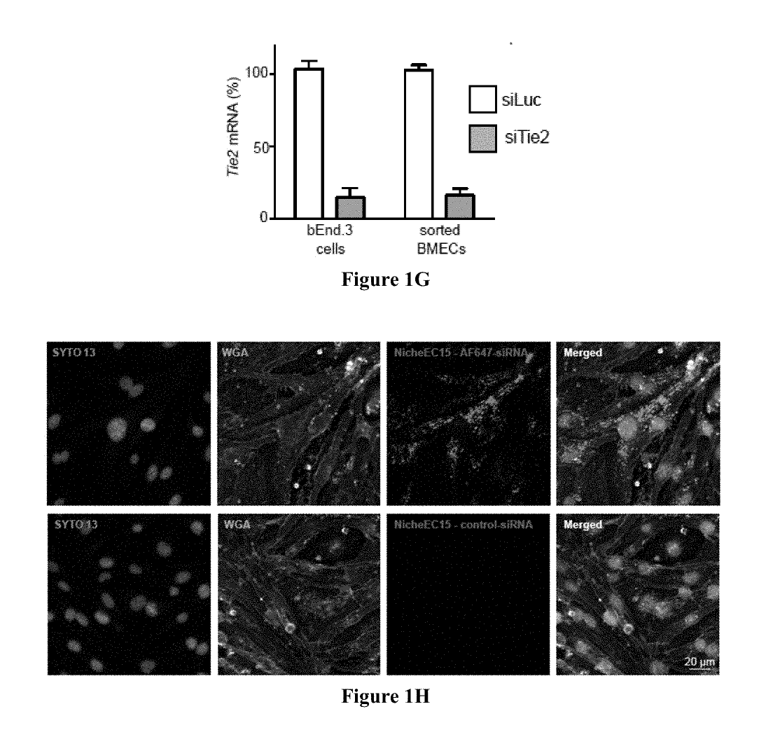

Bone marrow endothelial cells (BMEC) are an integral component of the hematopoietic niche and instruct stem cell and leukocyte behavior. Cell-specific deletion experiments revealed that both progenitor proliferation and migration are governed by BMEC-derived signals (1, 23). A polymer-lipid hybrid material was prepared by reacting C15 epoxide-terminated lipids with low molecular weight polyamines (PEI600) at a 14:1 molar ratio (FIG. 1A). The resulting material was combined with small interfering RNA (siRNA) and a polyethylene glycol (PEG)-lipid conjugate in a high-throughput microfluidic mixing chamber (24) to formulate nanoparticles via electrostatic interactions between the cationic polymeric material and the negatively charged nucleic acid (FIG. 1B). This nanomaterial was used as a starting point for testing different PEG surface coatings that alter biodistribution and pharmacokinetics of various nanoparticle types (25, 26). It is hypothesized that modulating nanoparticle PEG architecture enhances nanoparticle siRNA delivery to the bone marrow. By altering the PEG architecture, a library of 15 nanoparticle formulations was created, with three key parameters of the PEG-lipid conjugate modified in each candidate nanoparticle: i) molecular weight of the PEG surface coating (molecular weight range: 2000-5000), ii) PEG surface density, which was altered through varying the overall molar percentage of PEG within the formulation (molar percentage range: 2%-20%) and iii) the length of the lipid chain that anchors PEG within the nanoparticle membrane (FIG. 1C). This mini-library was injected intravenously into mice (1.0 mg/kg body weight) and then assessed Tie2 silencing in the bone marrow. Using a branched DNA assay, the best nanoparticle formulation (termed NicheEC-15) was indentified, which induced ˜80% Tie2 in vivo gene silencing in bone marrow (FIG. 1D). Cryoscanning electron microscopy and dynamic light scattering showed that NicheEC-15 formed multilamellar nanoparticles (FIG. 1E) with a 60-80 nm diameter (FIG. 1F). NicheEC-15 for Tie2 silencing in endothelial cells in vitro was next assessed. Nanoparticles containing 60 nM Tie2 siRNA induced potent gene silencing in the mouse endothelial cell line bEnd.3 and in primary murine BMEC (FIG. 1G). Confocal microscopic imaging confirmed efficient in vitro uptake of nanoparticles containing fluorescent siRNA (FIG. 1H).

### Example 2

### In Vivo Behavior of the Lead Particle NicheEC-15

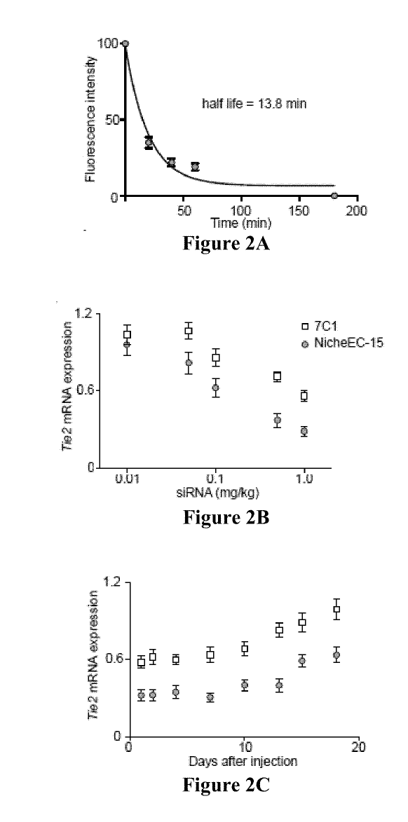

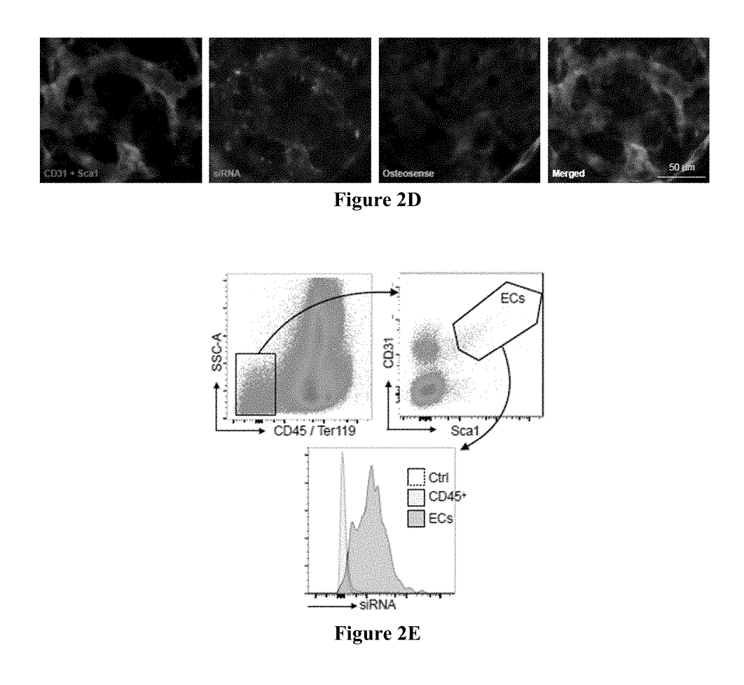

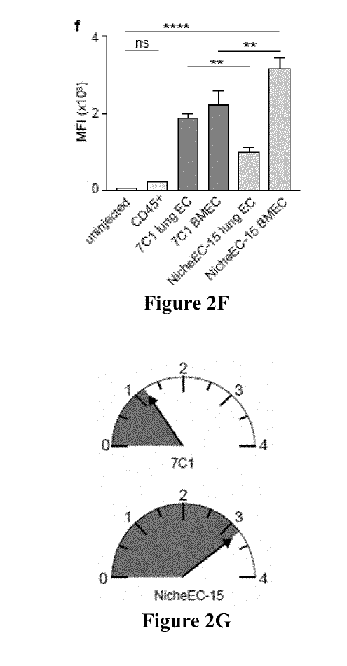

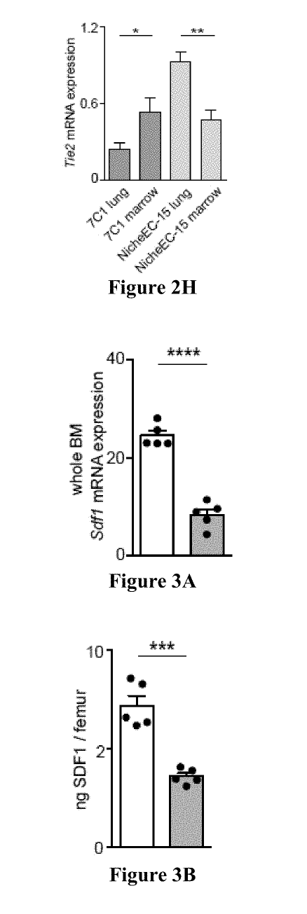

The in vivo blood half life of NicheEC-15, containing a fluorescent siRNA, was measured to be 13.8 minutes by fitting the decrease in blood fluorescence intensity over time after a single i.v. injection (FIG. 2A). Using a range of 0.01 to 1.0 mg/kg siTie2, dose-dependent knock down of Tie2 mRNA expression by qPCR was observed (FIG. 2B). At a dose of 1 mg/kg siTie2, the knock down achieved with NicheEC-15 was about 50% stronger than 7C1 nanoparticles (17), a previously described nanoparticle with excellent silencing in endothelial cells. A single injection of NicheEC-15 containing 1.0 mg/kg siTie2 induced long-lasting knock down for more than two weeks (FIG. 2C). To better understand the uptake patterns of NicheEC-15, the hematopoietic niche using intravital microscopy of the skull bone marrow was directly visualized. The vasculature was stained by intravenously injecting a cocktail of PE labeled CD31 and Sca1 antibodies, while Osteosense, a molecular imaging probe that enriches in osteoblasts, outlined the bone surface surrounding hematopoietic niches (27). Two hours after injecting NicheEC-15- containing fluorescently tagged siRNA, the siRNA associated imaging signal in the bone marrow vasculature was found (FIG. 2D). Cellular distribution of NicheEC-15 was measured by flow cytometric analysis of the bone marrow. While the highest fluorescence intensity in CD45− CD31+ Sca-1+ endothelial cells, nanoparticle uptake into CD45+ leukocytes was minimal was observed (FIGS. 2E and 2F). NicheEC-15 to 7C117 in vivo was benchmarked next. Compared to 7C1, NicheEC-15 uptake into BMEC increased significantly while uptake into lung endothelial cells decreased (FIG. 2F). While 7C1 uptake was similar in both organs, NicheEC-15 exhibited a three-fold higher uptake into bone marrow endothelial cells (FIG. 2G), a result that supports NicheEC-15's high avidity for the marrow over the lung. Furthermore, direct comparing Tie2 in lung and bone marrow showed that NicheEC-15 enhanced gene silencing in bone marrow while reducing such effects in lung, compared to 7C1 (FIG. 2H). Such a predilection may allow for high silencing efficiency in the marrow at lower doses.

### Example 3

### Sdf-1/Mcp-1 Target Screening

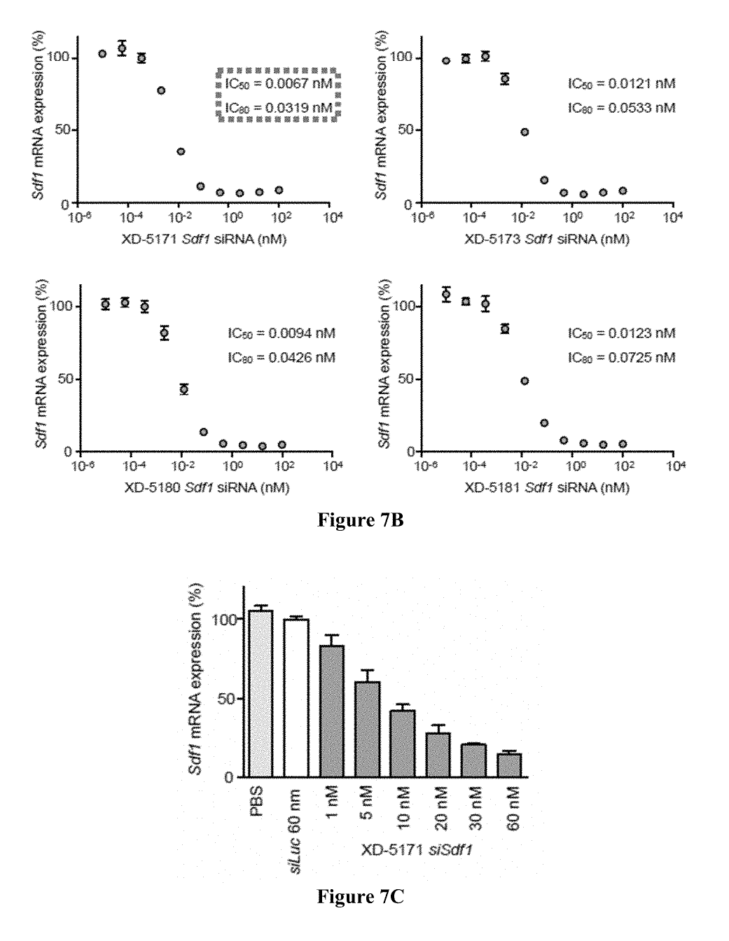

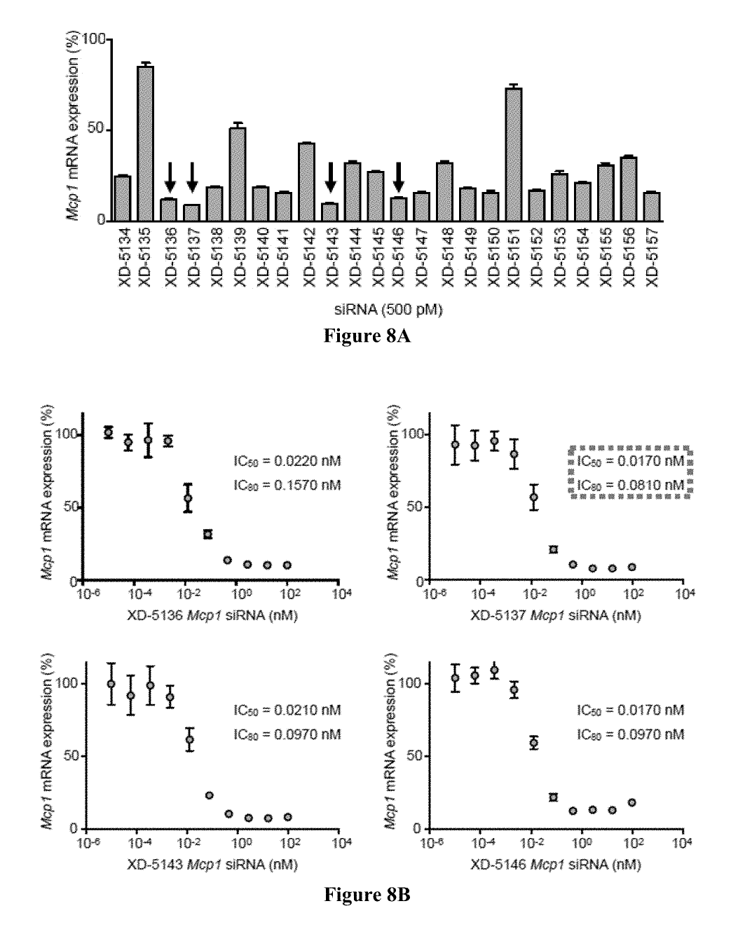

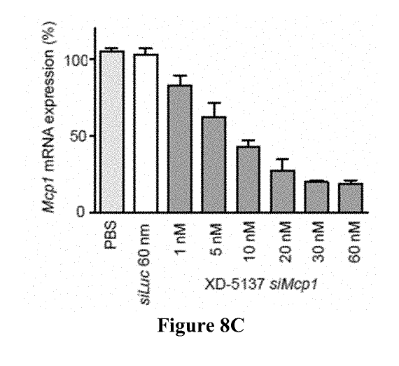

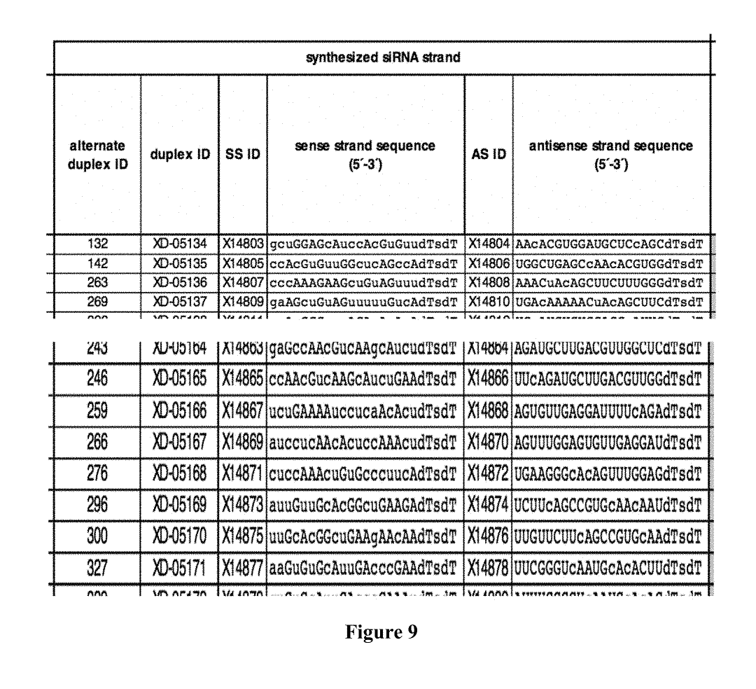

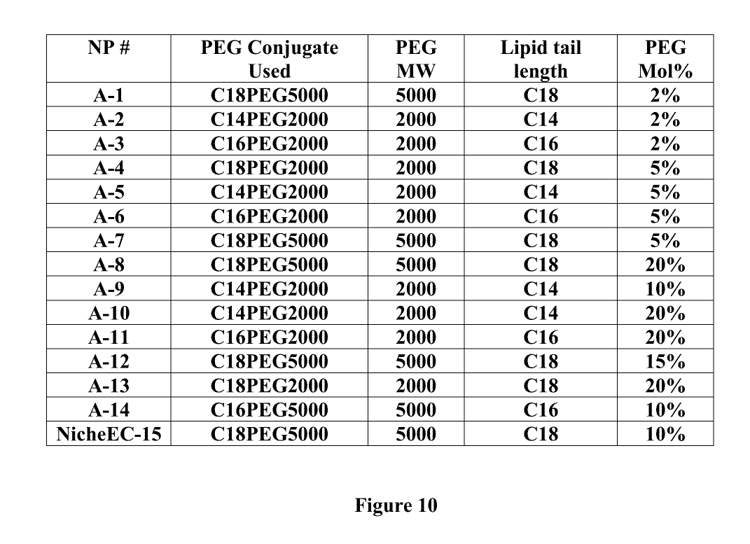

Having identified NicheEC-15 as a promising delivery material, implementation of RNAi for modulating the hematopoietic bone marrow niche was advanced. Two target proteins that influence hematopoietic stem cell and leukocyte behavior were selected, specifically stromal derived factor 1 (Sdf-1, also known as Cxcl12) and monocyte chemotactic protein 1 (Mcp-1, also known as Ccl-2). These well-studied proteins (1, 23, 28, and 29) were chosen because their known functional properties allowed for the use of specific functional in vivo assays; additionally, because these proteins control cellular quiescence and migration, they are interesting drug targets. Viable siRNA sequences targeting Sdf-1 (FIGS. 7A to 7C) and Mcp-1 (FIGS. 8A to 8C) were identified by in vitro screening of in silico-predicted candidates. The top four siRNA sequences silencing Sdf-1 (FIGS. 7A and 7B) and Mcp-1 (FIGS. 8A and 8B) were then screened in vitro with a dose response assay. The siRNAs with the lowest IC50 and IC80 were selected for scale-up, nanoparticle formulation and modification to minimize immunostimulation and off-target gene silencing (30). The lead siRNA candidates encapsulated in NicheEC-15 nanoparticles induced potent, dose-dependent gene silencing of Sdf-1 (FIG. 7C) and Mcp-1 (FIG. 8C) at low dosages in endothelial cells in vitro.

### Example 4

### siSdf1 Triggers Bone Marrow Cell Release

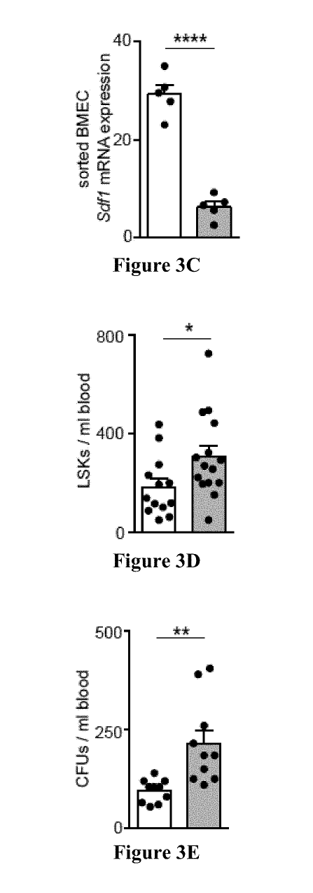

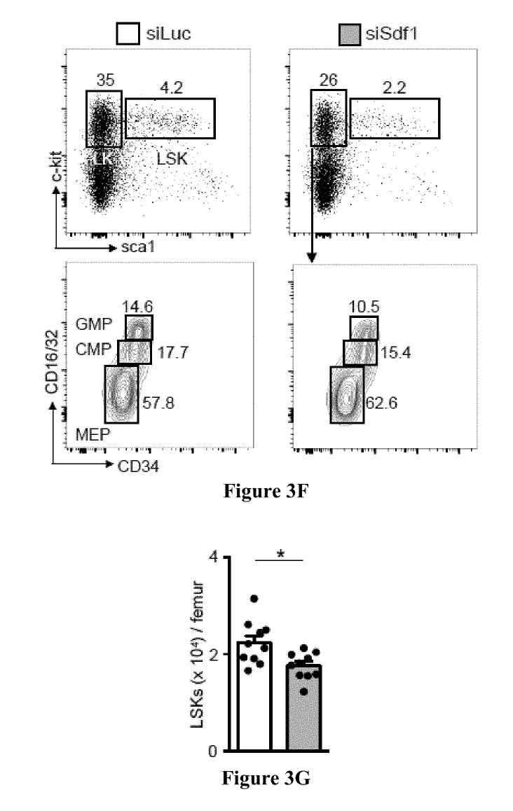

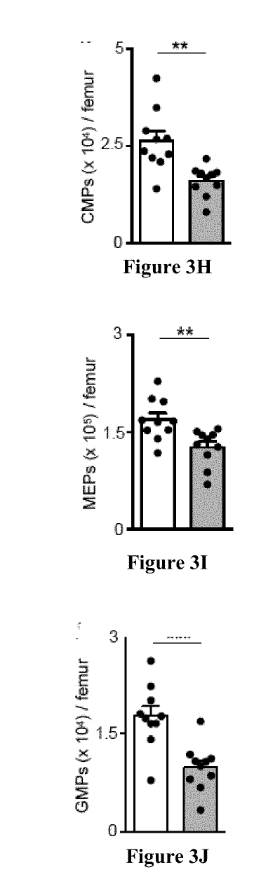

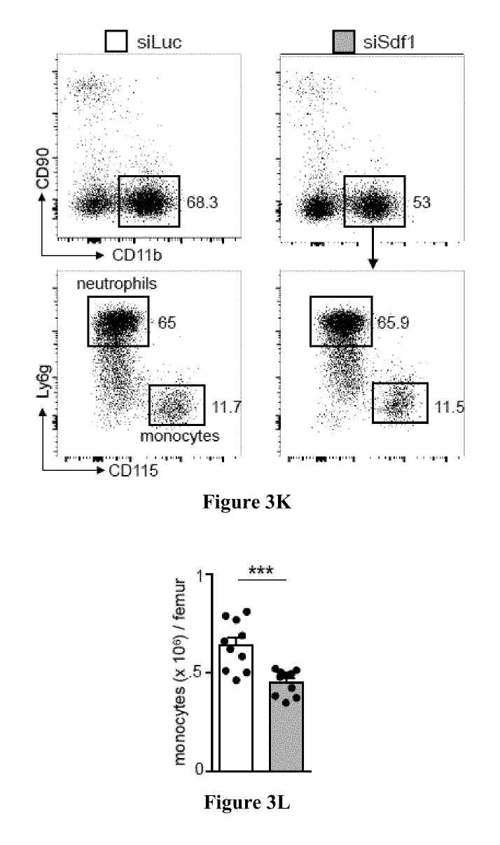

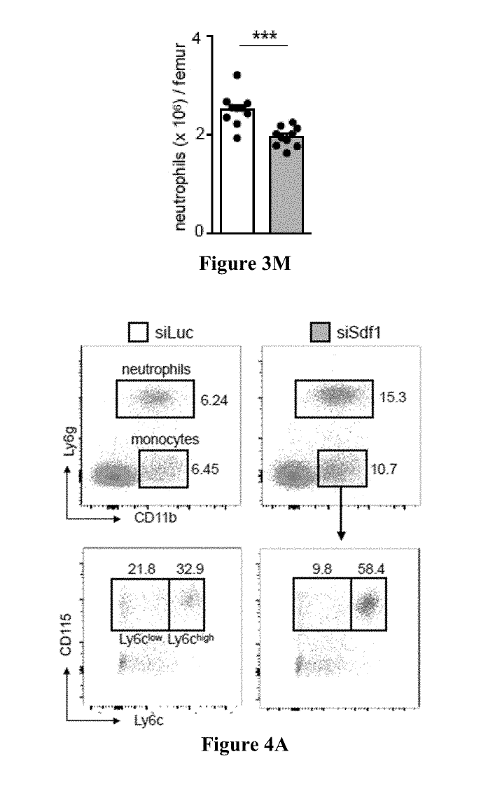

After tail vein injection of NicheEC-15 loaded with siRNA cargo targeting Sdf-1 (siSdf1), a >2-fold decrease in Sdf-1 expression was found (FIG. 3A) and significantly decreased Sdf-1 protein in the femur (FIG. 3B). To explore the cellular target responsible for this effect, bone marrow endothelial cells from mice after treatment with NicheEC-15 containing siSdf1 was FACS-isolated. Indeed, compared to controls, Sdf-1 expression was strongly reduced in these cells (FIG. 3C). Since Sdf-1 retains hematopoietic stem and progenitor cells (28) and leukocytes (29) in the bone marrow, HSPC migration was investigated to monitor functional consequences of RNAi. Indeed was investigated, and it was observed that there were higher lineage-sca1+ckit+ (LSK) cell numbers in blood of mice treated with siSdf1 (FIG. 3D), indicating release of these progenitor cells from the marrow. The increase in circulating HSPC was confirmed by colony forming unit (CFU) assays in blood (FIG. 3E). Accordingly, siSdf1 injection decreased bone marrow LSK cells (FIGS. 3F and 3G). Likewise, downstream myeloid progenitors, including common myeloid progenitors (CMP) and granulocyte macrophage progenitors (GMP), declined in numbers in the femur after siSdf1 treatment (FIGS. 3H to 3J).

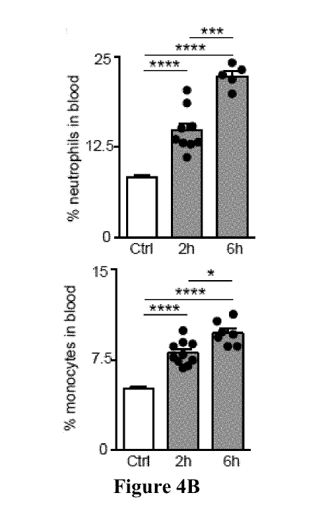

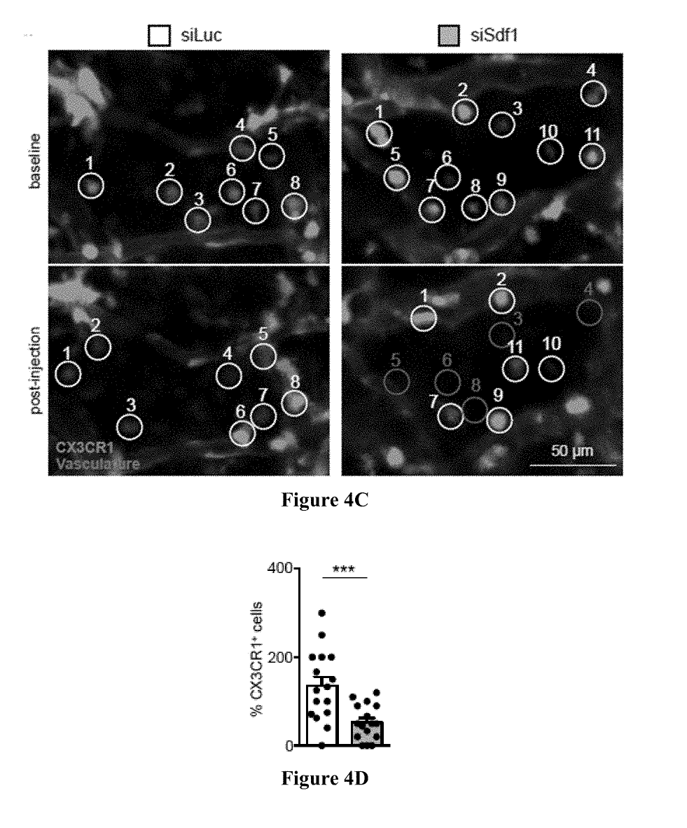

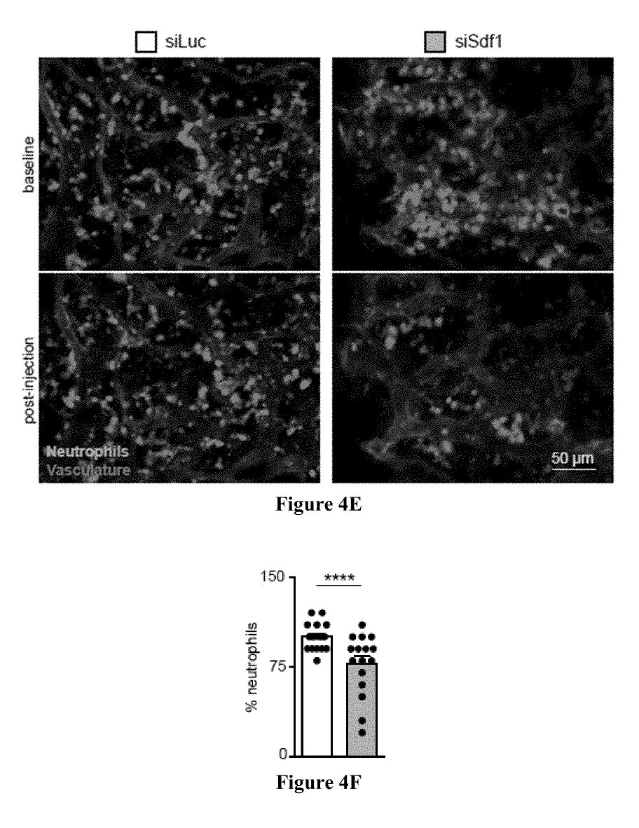

In addition to progenitor release, it was further detected that siSdf1 treatment leads to monocyte and neutrophil departure from the bone marrow (FIG. 3K to 3M). As a consequence, neutrophils and monocytes may become more numerous in circulation. Since the level of circulating myeloid cells regulates their presence at sites of inflammation and closely associates with patient mortality, the early dynamics of this leukocyte release were studied. Two hours after siSdf1 injection, myeloid cells became significantly more abundant compared to baseline, and increased even further at six hours (FIGS. 4A and 4B). Such a boost in circulating leukocytes may also be caused by redistribution from other cell pools, e.g. in the lung or spleen. Serial pre- and post-RNAi treatment intravital microscopy was used to survey the skull bone marrow. Bone marrow macrophages and monocytes in Cx3cr1^{GFP/+} mice before and up to 2.5 hours after siSdf1 injection was visualized. By monitoring specific bone marrow niches, individual cell behavior was tracked over time (FIG. 4C). Treatment with siSdf1 triggered departure of Cx3cr1+ cells, while this was not observed after treatment with control siRNA (FIG. 4D). A similar dynamic was observed for neutrophils that were labeled in vivo with an Ly6g-APC antibody (FIGS. 4E and 4F). In sum, these data demonstrate the ability to trigger stem cell and leukocyte release by silencing retention factors in the bone marrow vasculature.

### Example 5

### siMcp1 Inhibits Bone Marrow Cell Release

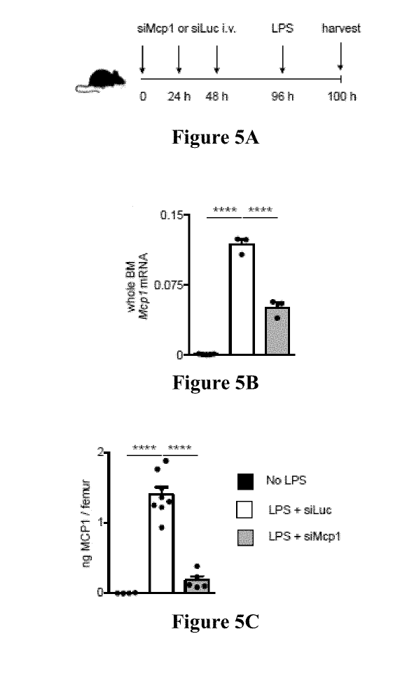

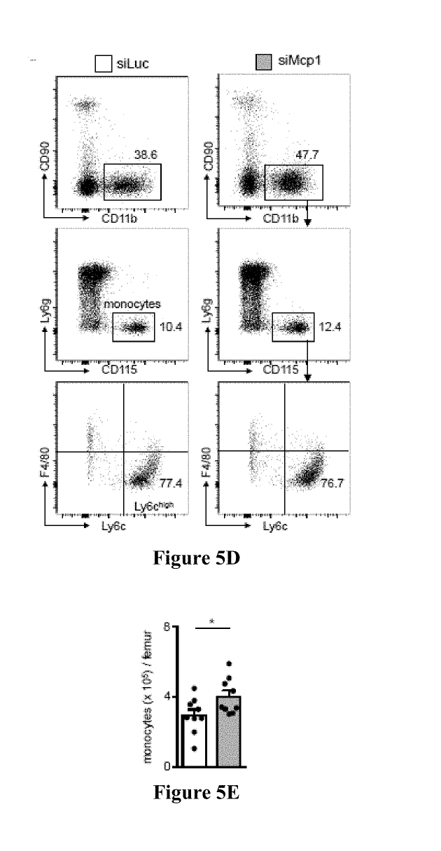

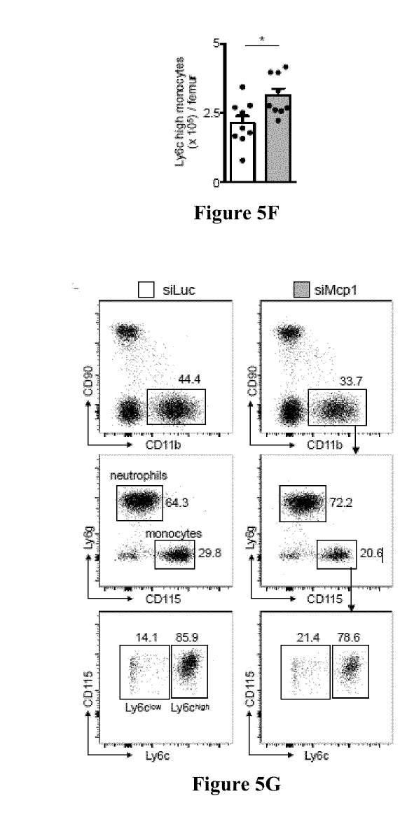

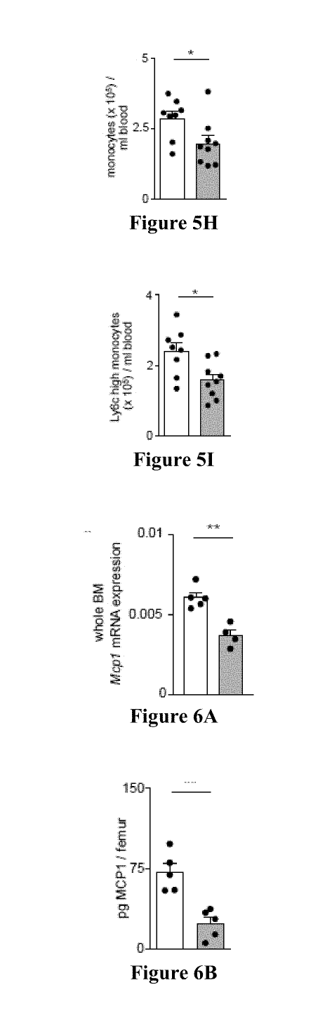

While Sdf-1 silencing increased cell migration from the marrow, a gene target was next chosen to explore the opposite functionality, i.e. to use RNAi to dampen leukocyte traffic. Such an intervention could be a therapeutic avenue to reducing systemic leukocyte supply in the setting of inflammation. Monocyte bone marrow departure is triggered by secretion of the chemokine Mcp-1 into the sinusoidal space, for instance after infection, injection of lipopolysaccharide, a building block of the bacterial wall that binds toll like receptors, or myocardial infarction (16, 31). Because monocytes express the cognate chemokine receptor Ccr2, they follow the Mcp-1 chemokine gradient and extravasate from the niche into the bone marrow sinusoids. The effect of Mcp-1 silencing in the setting of LPS-induced inflammation was tested (FIG. 5A). As expected, LPS injection dramatically increased Mcp1 expression in the marrow, and such expression was significantly reduced by siMcp1 treatment (FIG. 5B). Mcp1 protein levels were similarly elevated by LPS, which was abrogated by siMcp1 treatment (FIG. 5C). As a consequence, monocyte numbers in the bone marrow were higher in siMcp1-treated mice (FIG. 5D to 5F), whereas blood monocyte numbers decreased (FIG. 5G to 5I).

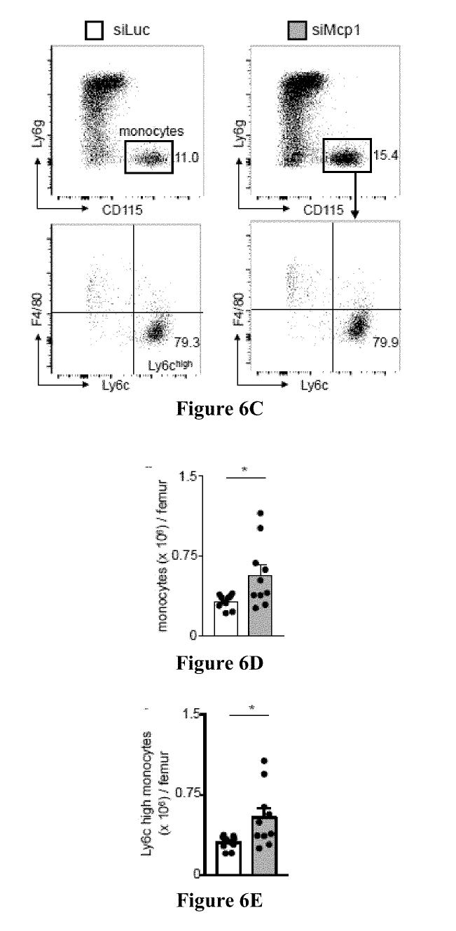

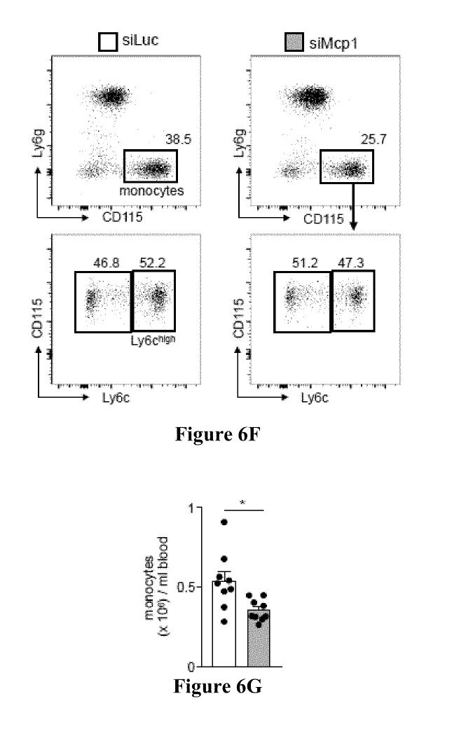

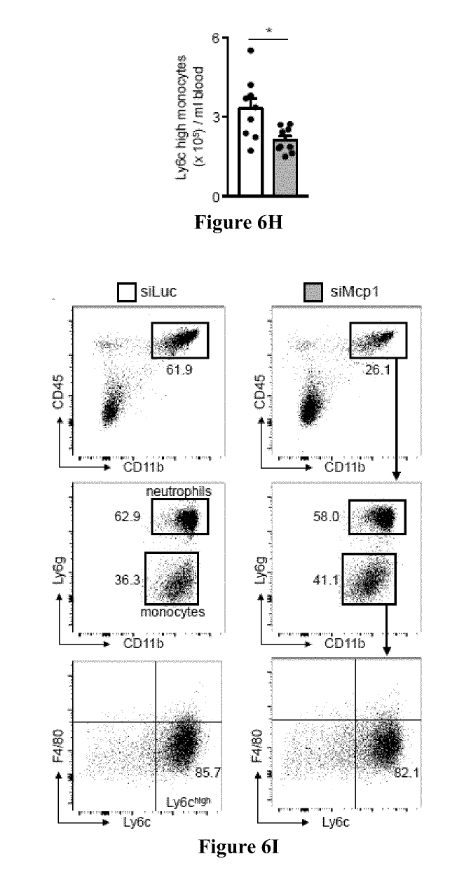

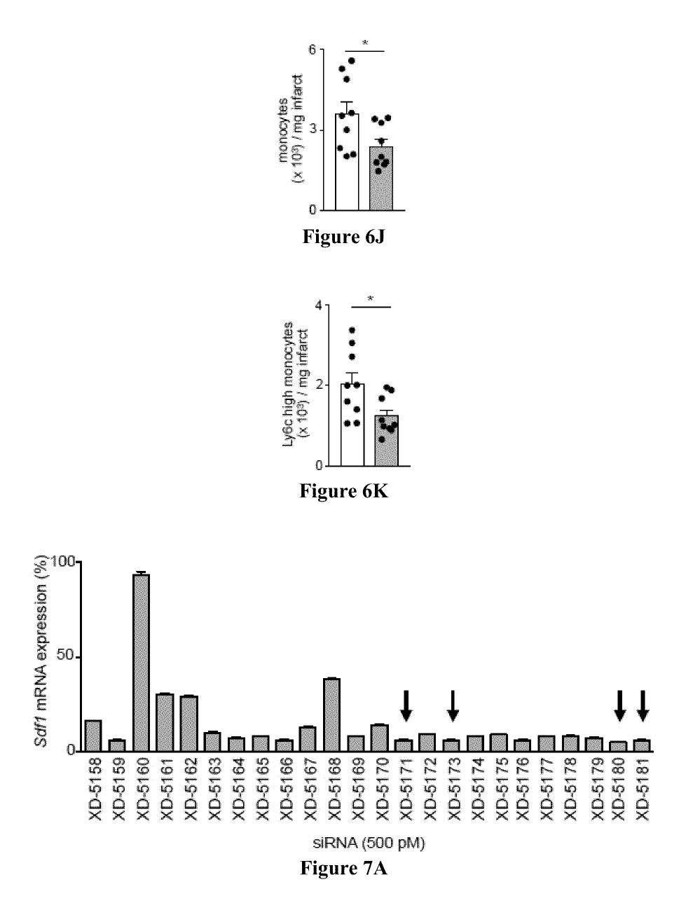

Mcp-1/Ccr2 signaling also regulates monocyte migration in sterile inflammation, including during cardiovascular disease. In particular, acute myocardial infarction (MI) triggers blood monocytosis, followed by cell recruitment into the ischemic myocardium (32). After MI, systemic oversupply of inflammatory monocytes may lead to heart failure and subsequent ischemic events (8). Therefore it was tested if silencing Mcp1 in mice with acute MI may reduce inflammatory Ly6c^{high} monocyte numbers in circulation and the heart. Treating mice with siMcp1 led to a lower bone marrow Mcp1 expression (FIG. 6A) and protein level (FIG. 6B). Consequently, monocyte bone marrow numbers rose (FIGS. 6C and 6D) while circulating monocyte numbers remained lower (FIG. 6F to 6H), results that indicated attenuated cell release from the marrow. Ultimately, fewer monocytes, including the inflammatory Ly6c^{high} subset, were recruited to the infarcted heart in mice treated with siMcp1 (FIGS. 6I to 6K).

### Methods

For compositions with the nanoparticle comprising a conjugated lipomer of Formula (I) and a lipid-polyethylene glycol (PEG) conjugate of Formula (II), these compositions of both components were made via microfluidics-based synthesis, where these two materials complex with nucleic acids via electrostatic interactions to form nanoparticles. For a particle comprising a conjugated lipomer of Formula (I) and a lipid-polyethylene glycol (PEG) conjugate of Formula (II), this particle is synthesized using microfluidic devices to utilize chaotic mixing to form nanoparticles via electrostatic interactions.

### Example 6

### Polymer-Lipid Hybrid Synthesis

Polymer-lipid hybrids were synthesized by reacting C15 alkyl epoxides (Tokyo Chemical Industry and Sigma) with PEI600 at 90° C. in 100% ethanol for 48-72 hours at a 14:1 molar ratio. Due to the polydispersity of PEI600 (Mn=600, PDI=0.33), polymer-lipids were purified via high performance liquid chromatography (HPLC) on a silica column with DCM. MeOH and NH₄OH were added to the solvent over a 45 minute period to decrease polarity, which enabled HPLC separation of the polymer-lipid into fractions related to the hydrophobic C15: hydrophilic PEI ratio. Mixtures were split into five fractions and individually assessed for their ability to induce gene silencing in mice.

### Example 7

### siRNA Nanoparticle Synthesis

To formulate nanoparticles, polymer-lipid materials were combined with polyethylene (PEG)-lipid conjugates (Avanti Polar Lipids) and mixed with siRNA in a microfluidic device. Polymer-lipids and PEG-lipid conjugates were dissolved in 200 proof ethanol and loaded into a gas-tight Hamilton glass syringe, while siRNA were in an 10 mM citrate buffer (pH 3.0; Teknova). Syringes were connected to inlets of a microfluidic device containing static mixers, and contents were perfused through the device to formulate nanoparticles. Nanoparticle formulations were sterile filtered against PBS using a MW 20,000 cutoff dialysis cassette (Thermo Scientific) to remove citrate and ethanol. Nanoparticle size and structure were determined by both dynamic light scattering (DLS) using a Zetasizer Nano ZS machine (Malvern Instruments) and cryo-transmission electron microscopy (TEM) using a Tecnai 12 G2 TEM (FEI). Nanoparticle samples were prepared in a vitrification system (25° C., ˜100% humidity), and images of samples were recorded on an UltraScan 1000 CCD camera (Gatan) in low dose conditions. For zeta potential measurements, nanoparticles (25 μL) at a polymer-lipid concentration of 1 mg/mL were added to PBS (1 mL) and measured using a Malvern Zetasizer Nano ZS machine. The concentration of siRNA used for in vitro and in vivo treatment was quantified using both a QuaRibogreen assay (Invitrogen) and NanoDrop measurement (Thermo Scientific).

### Example 8

### Cell Culture Experiments

The endothelial cell line bEnd.3 (CRL-2299, ATCC) was cultured in Dulbecco's modified Eagle's medium, supplemented with 10% (vol/vol) fetal bovine serum and 1% (vol/vol) penicillin/streptomycin under humidified conditions at 37° C. and 5% CO₂ until cell confluence. To investigate knock down efficiency, bEnd.3 cells were plated in 24 well plates (150,000 cells per well) and incubated for 24 hours prior to treatment with nanoparticles formulated with siRNA targeting one of the following genes: luciferase (LUC), monocyte chemoattractant protein-1 (MCP-1) or stromal-derived factor-1 (SDF-1). A serial dilution of each siRNA nanoparticle formulation in PBS was prepared at concentrations of 1-60 nM of siRNA. Samples were then incubated for 24 hours prior to gene expression analysis. Cells were washed with PBS and harvested using 0.25% trypsin. RNA was extracted from cells using a Arcturus PicoPure RNA Isolation Kit (Thermo Scientific). To study the in vitro uptake of NicheEC-15 by microscopy, bEnd.3 cells were plated in 6 well plates (500,000 cells per well) and grown until confluence. NicheEC-15 containing Alexa Fluor 647 coupled luciferase siRNA was then added at a concentration of 200 nM RNA 2 hours before imaging. 15 minutes before imaging 5 μM Syto 13 Green (Life Technologies) and 5 μg/ml wheat germ agglutinin Alexa Fluor 555 (WGA, Life Technologies) were added. During the last 5 minutes the cell cultures were washed repeatedly with PBS. The controls were treated identically, except that NicheEC-15 containing unlabelled luciferase siRNA was used. All steps except washing were done under humidified conditions at 37° C. and 5% CO₂.

### Example 9

### Mice

All animal procedures conducted at MGH and MIT were approved by the Institutional Animal Care and Use Committees (IACUC) and were in accordance with local, state and federal regulations. C57BL/6J and hemizygous Cx3cr1GFP/+ mice were used at 11 to 13 weeks of age. C57BL/6J (stock #000664) mice were purchased and for hemizygous Cx3cr1^{GFP/+} mice, homozygous B6.129P-Cx3cr1^{tm1Litt/J} (stock #005582) mice were crossed with C57BL/6J mice, both from Jackson Laboratory. Nanoparticles were administered by tail vein injection. Myocardial infarction was induced by permanent coronary ligation as previously described (27). Briefly, mice were anaesthetised, given buprenorphin subcutaneously for analgesia, intubated and ventilated with 2% isoflurane supplemented with oxygen. After thoracotomy, the heart was exposed, and the left coronary artery was identified, and permanently ligated with a monofilament nylon 8-0 suture. For the LPS model, after nanoparticle treatment, 10 μl normal saline containing 2 ng LPS (Sigma) was injected i.v. and mice harvested 4 hours after LPS injection.

### Example 10

### Tie2 Gene Silencing

Gene silencing in femoral bone marrow was assessed up to 18 days post injection of NicheEC-15 containing a dose of 1.0 mg/kg Tie2 siRNA. For dose response studies, ice were treated with a single dosage of Tie2 siRNA nanoparticles ranging from 0.01-1.0 mg/kg, and femurs were harvested 48 h post-injection. Mice were sacrificed by CO₂ asphyxiation, and femurs were harvested and immediately snap-frozen in liquid nitrogen. Frozen tissues were pulverized to form a powder using a SPEX 2010 Geno/Grinder (SPEX SamplePrep). Tissue lysates were prepared in Tissue and Cell Lysis Buffer (Epicentre) supplemented with 0.5 mg/mL Proteinase K (Epicentre). Tissue samples were mixed at 1400 RPM for 2 h at 65° C. and centrifuged at 16,000 RCF to remove bone debris. mRNA levels in the supernatant were quantified using the QuantiGene 2.0 luminescent-based branched DNA (bDNA) assay kit and the QuantiGene 2.0 probes against Tie2 and GAPDH (Thermo Fisher Scientific) according to the manufacturer's protocol. Luminescent signals were measured using a Tecan Infinite 200 PRO plate reader (Tecan). Standard curves for femur tissues and each target gene were constructed using samples from untreated mice to ensure optimal dilutions for assay samples that avoid luminescent signal saturation. Tie2 silencing in treated mice was quantified by calculating the ratio of Tie2 gene luminescence to Gapdh gene luminescence, with all values normalized to Tie2:Gapdh gene ratios from untreated mice.

### Example 11

### mRNA Extraction and qPCR

Messenger RNA (mRNA) was extracted from cells using a Arcturus PicoPure RNA Isolation Kit (Thermo Scientific) for cultured and sorted endothelial cells, and the RNeasy Mini Kit (Qiagen) was used for whole bone marrow samples, both according to the manufacturers' protocols. mRNA concentration was measured using a NanoDrop 2000c Spectrophotometer (Thermo Scientific) or using a 2100 Bioanalyzer (Agilent) for low mRNA concentration. After adjusting for yield mRNA was transcribed to complementary DNA (cDNA) with the high capacity RNA to cDNA kit (Applied Biosystems). Taqman primers (Applied Biosystems) were used. Results were expressed by Ct values normalized to the housekeeping gene Gapdh.

### Example 12

### Flow Cytometry and Cell Sorting

Single-cell suspensions were obtained from peripheral blood, bone marrow, aorta and lung. Briefly, blood was collected by eye bleeding using heparinised capillaries or, if larger volumes were needed, by cardiac puncture and addition of 50 mmol/L EDTA. Red blood cells lysis was achieved by adding 1× red blood cell lysis buffer (Biolegend) for 2 minutes. After blood collection, mice were perfused through the left ventricle with 30 mL ice-cold PBS. Bone marrow was harvested by flushing femurs in PBS with 0.5% bovine serum albumin (BSA) for leukocyte and HSPC staining or with HBSS containing 2% FBS, 2 mg/ml disease 2 and 1 mg/ml collagenase IV and incubated with gentle agitation for 30 minutes at 37° C. for endothelial cells. Lungs were excised and minced with a fine scissor before digestion in DMEM w/o phenol red containing 2% FBS, 2 mg/ml dispase 2, 5 mg/ml collagenase I (Worthington) and 1 mg/ml DNase I (Sigma) at 37° C. and 125 revolutions for 20 minutes. Tissues were then plunged thorough 40-μm nylon mesh (BD Falcon), washed and centrifuged (8 minutes; 300 g; 4° C.). The obtained single cell suspensions were stained at 4° C. for 30 minutes and afterwards washed, centrifuged and resuspended. Fluorochrome- and biotin-conjugated antibodies specific to mouse B220 (clone RA3-6B2), CD3e (clone 145-2C11), CD4 (clone GK1.5), CD8a (clone 53-6.7), CD11b (clone M1/70), CD11c (clone HL3), CD16/32 (clone 93 and 2.4G2), CD19 (clone 1D3), CD31 (clone MEC13.3), CD34 (clone RAM34), CD41 (clone MWReg30), CD45.2 (clone 104), CD48 (HM48-1), CD90.2 (clone 53-2.1), CD115 (clone AFS98), CD150 (clone TC15-12F12.2), c-kit (clone 2B8), F4/80 (clone BM8), GR1 (clone RB6-8C5), IL7Rα (clone A7R34), Ly6C (clone AL-21), Ly-6G (clone 1A8), NK1.1 (clone PK136), Sca-1 (clone D7) and Ter-119 (clone TER-119) were used. Antibodies were purchased from Biolegend, BD Biosciences or eBioscience. Monocytes were identified as CD90− CD19− NK1.1− Ly-6G− D45.2+ CD11bhighCD115+ F4/80low and separated into Ly-6C^{low} and a Ly-6C^{high} populations. Neutrophils were identified as CD90− CD19− NK1.1− D45.2+ CD11bhigh Ly-6G+. Blood and bone marrow LSK were identified as (B220 CD3e CD4 CD8a CD11b CD11c CD19 CD90.2 GR1 IL7Ra NK1.1 Ter119)− c-kit+ Sca-1+, LKs as (B220 CD3e CD4 CD8a CD11b CD11c CD19 CD90.2 GR1 NK1.1 Ter119)− c-kit+ Sca-1− and subdivided into CMP, MEP and GMP by their CD16/32 and CD34 expression. Bone marrow and lung endothelial cells were gated as CD45.2− CD41− Ter119− CD31+ Sca-1+ and Ter119− CD45+ CD31+, respectively. To define positivity for AF647-siRNA uptake, cells from uninjected mice were used as a negative control. Data were acquired on an LSRII (BD Biosciences) and analysed with FlowJo software (Tree Star). For cell sorting the populations were defined as described and sorted using a FACSAria II cell sorter. The cells were directly sorted into lysis buffer, and the samples were vortexed, quick frozen on dry ice and stored at −80° C. until RNA extraction.

### Example 13

### Colony-Forming Unit Assay

Colony-forming unit (CFU) assays were performed using a semisolid cell culture medium (Methocult M3434, Stem Cell Technology) following the manufacturer's protocol. 100 μl whole blood were processed following the protocol, plated on 35-mm plates in duplicates and incubated for 10 days. Colonies were counted using a low-magnification inverted microscope.

### Example 14

### Intravital Microscopy

To visualize in vivo uptake of NicheEC-15 in bone marrow endothelial cells, the skull bone marrow was imaged as previously described (27). Osteosense was injected i.v. 24 hours, NicheEC-15 AF647-siRNA 2 hours and PE labeled CD31 and Sca1 antibodies 1 hour before imaging.

### Example 15

### ELISA

ELISAs for SDF1 and MCP1 (both R&D) in the bone marrow were performed by spinning (11.000 g, 2 min, 4° C.) the bone marrow out of one femur and taking the supernatant for ELISAs, which were performed according to the manufacturers' instructions.

### Example 16

### Statistics

Data are expressed as mean±SEM. Analyses were performed using Prism 7 (GraphPad Software Inc). For a 2-group comparison, a Student t test was applied if the pretest for normality (D'Agostino-Pearson normality test) was not rejected at 0.05 significance level; otherwise, a Mann-Whitney test for nonparametric data was used. ANOVA tests followed by Bonferroni post-tests were applied for comparison of >2 groups. P values <0.05 indicate statistical significance.

### Discussion

Bone marrow niche cells regulate hematopoietic and leukocyte activity in numerous ways, including cell transit into the circulation (1, 23, and 33). A nanoformulation capable of delivering siRNA to bone marrow niche cells was developed. These new nanoparticles are derivatives of prior materials with avidity for lung endothelial cells (17). Nanoparticle siRNA delivery to the bone marrow by modulating nanoparticle PEG architecture was enhanced, including i) the molecular weight of the PEG surface coating, ii) the length of the lipid chain that anchors PEG within the nanoparticle membrane and iii) PEG surface density. By screening polymer-lipid hybrid materials, combined with modulating PEG nanoparticle surface coatings, a nanoparticle, termed NicheEC-15, was developed with superior avidity for and efficient silencing in bone marrow endothelial cells. To test NicheEC-15, two prototypical hematopoietic niche factors were silenced. One of them, Sdf-1, promotes stem cell quiescence and bone marrow retention of leukocytes via interaction with its cognate receptor Cxcr4. This pathway is currently targeted clinically using G-CSF and AMD3100, which trigger stem cell liberation into the blood (34). Silencing HSPC retention factors using nanoparticle enabled RNAi may improve the cell yield in stem cell transplantation, especially in patients that are poor stem cell mobilizers due to comorbidities such as diabetes (35). The second target, Mcp-1, has the opposite effect, as this chemokine triggers bone marrow monocyte release. It was found that silencing those proteins, using NicheEC-15 enabled RNAi, altered migration of HSPC and leukocytes from bone marrow niches into the systemic blood pool. The data provides first proof-of-principle that bone marrow HSPC and leukocyte behavior may be addressed using RNAi therapeutics with avidity for the hematopoietic niche.

Due to RNAi's modular character, this approach can be adapted for silencing any other endothelial cell derived bone marrow niche factor, including growth hormones, cytokines and adhesion molecules that influence HSPC or leukocyte biology. Furthermore, silencing receptors may modulate endothelial cells' ability to sense and relay information they receive from the circulation. The function of bone marrow niche cells in steady state and disease is the subject of vibrant research, and large unbiased data-sets, for instance obtained by RNA sequencing of bone marrow endothelial cells, are increasingly becoming available (36). The technology described herein is ideally suited to rapidly testing the function of highly expressed genes and newly arising drug candidates in vivo.

Endothelial cells separate bone marrow niches from the blood pool. Their barrier function, which ultimately regulates cell release, make them a particularly attractive target for modulating cell migration. However, there are other niche cells that regulate hematopoiesis. Future materials may deliver siRNA to bone marrow macrophages, mesenchymal stromal cells or osteoblasts, thereby targeting other niche components. Such additions to the target cell portfolio may also facilitate RNAi for niche-driven malignancies.

Stem cell quiescence and lineage bias are of particular interest as they decisively influence the size and function of any systemic blood cell pool. As demonstrated by silencing Mcp-1, reducing the release of short-lived leukocytes from their site of production may dampen inflammation at any inflammatory site, including the ischemic heart. Thus, reducing the oversupply of innate immune cells may curtail exuberant inflammatory actiuvity in cardiovascular disease (8), the leading cause of death worldwide.

### REFERENCES

1. Morrison, S. J. & Scadden, D. T. The bone marrow niche for haematopoietic stem cells. Nature 505, 327-334 (2014).
2. Crane, G. M., Jeffery, E. & Morrison, S. J. Adult haematopoietic stem cell niches. Nature Reviews Immunology 17, 573-590 (2017).
3. Kaplan, R. N., Psaila, B. & Lyden, D. Niche-to-niche migration of bone-marrow-derived cells. Trends in Molecular Medicine 13, 72-81 (2007).
4. Sipkins, D. A. et al. In vivo imaging of specialized bone marrow endothelial microdomains for tumour engraftment. Nature 435, 969-973 (2005).
5. Katayama, Y. et al. Signals from the Sympathetic Nervous System Regulate Hematopoietic Stem Cell Egress from Bone Marrow. Cell 124, 407-421 (2006).
6. Gratwohl, A. et al. Hematopoietic stem cell transplantation: a global perspective. JAMA 303, 1617-1624 (2010).
7. Giralt, S. et al. Optimizing autologous stem cell mobilization strategies to improve patient outcomes: consensus guidelines and recommendations. Biology of blood and marrow transplantation: journal of the American Society for Blood and Marrow Transplantation 20, 295-308 (2014).
8. Swirski, F. K. & Nahrendorf, M. Leukocyte behavior in atherosclerosis, myocardial infarction, and heart failure. Science 339, 161-166 (2013).
9. Calado, R. T. & Young, N. S. Telomere maintenance and human bone marrow failure. Blood 111, 4446-4455 (2008).
10. Seo, A. et al. Bone marrow failure unresponsive to bone marrow transplant is caused by mutations in THPO. Blood (2017).
11. Whitehead, K. A., Langer, R. & Anderson, D. G. Knocking down barriers: advances in siRNA delivery. Nature Reviews Drug Discovery 8, 129-138 (2009).
12. Titze-de-Almeida, R., David, C. & Titze-de-Almeida, S. S. The Race of 10 Synthetic RNAi-Based Drugs to the Pharmaceutical Market. Pharmaceutical research 34, 1339-1363 (2017).
13. Anselmo, A. C. & Mitragotri, S. Nanoparticles in the Clinic-Anselmo-Bioengineering &amp; Translational Medicine—Wiley Online Library. Bioengineering &amp; Translational . . . (2016).
14. Yin, H. et al. Non-viral vectors for gene-based therapy. Nature reviews. Genetics 15, 541-555 (2014).
15. Kanasty, R., Dorkin, J. R., Vegas, A. & Anderson, D. Delivery materials for siRNA therapeutics. Nature Materials 12, 967-977 (2013).
16. Leuschner, F. et al. Therapeutic siRNA silencing in inflammatory monocytes in mice. Nature Biotechnology 29, 1005-1010 (2011).
17. Dahlman, J. E. et al. In vivo endothelial siRNA delivery using polymeric nanoparticles with low molecular weight. Nature Nanotechnology 9, 648-655 (2014).
18. Dong, Y. et al. Lipopeptide nanoparticles for potent and selective siRNA delivery in rodents and nonhuman primates. Proc Natl Acad Sci USA 111, 3955-3960 (2014).
19. Love, K. T. et al. Lipid-like materials for low-dose, in vivo gene silencing. Proc Natl Acad Sci USA 107, 1864-1869 (2010).
20. Khan, O. F. et al. Ionizable Amphiphilic Dendrimer-Based Nanomaterials with Alkyl-Chain-Substituted Amines for Tunable siRNA Delivery to the Liver Endothelium In Vivo. Angewandte Chemie (International ed. in English) (2014).
21. Khan, O. F. et al. Dendrimer-Inspired Nanomaterials for the in Vivo Delivery of siRNA to Lung Vasculature. Nano Letters (2015).
22. Whitehead, K. A. et al. Degradable lipid nanoparticles with predictable in vivo siRNA delivery activity. Nature communications 5, 4277 (2014).
23. Mendelson, A. & Frenette, P. S. Hematopoietic stem cell niche maintenance during homeostasis and regeneration. Nat Med 20, 833-846 (2014).
24. Chen, D. et al. Rapid discovery of potent siRNA-containing lipid nanoparticles enabled by controlled microfluidic formulation. Journal of the American Chemical Society 134, 6948-6951 (2012).
25. Gref, R. et al. &apos;Stealth&apos; corona-core nanoparticles surface modified by polyethylene glycol (PEG): influences of the corona (PEG chain length and surface density) and of the core composition on phagocytic uptake and plasma protein adsorption. Colloids and surfaces. B, Biointerfaces 18, 301-313 (2000).
26. Owens III, D. E. & Peppas, N. A. Opsonization, biodistribution, and pharmacokinetics of polymeric nanoparticles. International journal of pharmaceutics 307, 93-102 (2006).
27. Heidt, T. et al. Chronic variable stress activates hematopoietic stem cells. Nat Med 20, 754-758 (2014)
28. Cheng, M. & Qin, G. Progenitor cell mobilization and recruitment: SDF-1, CXCR4, α4-integrin, and c-kit. Progress in molecular biology and translational science 111, 243-264 (2012).
29. Furze, R. C. & Rankin, S. M. The role of the bone marrow in neutrophil clearance under homeostatic conditions in the mouse. The FASEB Journal 22, 3111-3119 (2008).
30. Whitehead, K. A., Dahlman, J. E., Langer, R. S. & Anderson, D. G. Silencing or Stimulation? siRNA Delivery and the Immune System. Annual Review of Chemical and Biomolecular Engineering 2, 77-96 (2011).
31. Serbina, N. V. & Pamer, E. G. Monocyte emigration from bone marrow during bacterial infection requires signals mediated by chemokine receptor CCR2. Nat Immunol 7, 311-317 (2006).
32. Nahrendorf, M., Pittet, M. J. & Swirski, F. K. Monocytes: protagonists of infarct inflammation and repair after myocardial infarction. Circulation 121, 2437-2445 (2010).
33. Itkin, T. et al. Distinct bone marrow blood vessels differentially regulate haematopoiesis. Nature 532, 323-328 (2016).
34. Broxmeyer, H. E. et al. Rapid mobilization of murine and human hematopoietic stem and progenitor cells with AMD3100, a CXCR4 antagonist. The Journal of Experimental Medicine 201, 1307-1318 (2005).
35. Ferraro, F. et al. Diabetes impairs hematopoietic stem cell mobilization by altering niche function. Sci Transl Med 3, 104ra101 (2011).
36. Langen, U. H. et al. Cell-matrix signals specify bone endothelial cells during developmental osteogenesis. Nat Cell Biol 19, 189-201 (2017).

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A composition comprising:
a conjugated lipomer of Formula (I): 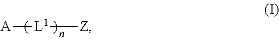
or a pharmaceutically acceptable salt thereof,
wherein:
each instance of L¹ is independently selected from the formulae: 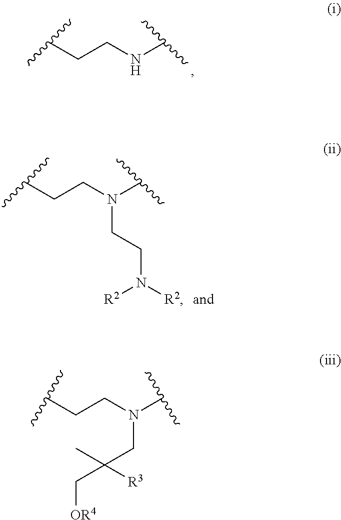
provided that at least one L¹ is selected from formula (iii);
n is an integer between 3 to 45, inclusive;
each instance of R² is independently hydrogen; acyl; silyl; sulfonyl; an amino protecting group; substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; substituted or unsubstituted carbocyclyl; substituted or unsubstituted heterocyclyl; substituted or unsubstituted aryl; substituted or unsubstituted heteroaryl; a substituted or unsubstituted polyethyleneimine; or a group of the formula (iii′): 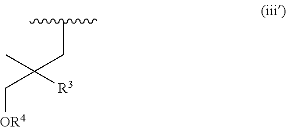
or the two R² groups are joined to form a substituted or unsubstituted heterocyclyl;
each instance of R³ is independently substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; substituted or unsubstituted carbocyclyl; substituted or unsubstituted heterocyclyl; substituted or unsubstituted aryl; substituted or unsubstituted heteroaryl; or a hydrophilic polymer;
each instance of R⁴ is independently hydrogen, acyl; silyl; a hydroxyl protecting group; substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; substituted or unsubstituted carbocyclyl; substituted or unsubstituted heterocyclyl; substituted or unsubstituted aryl; or substituted or unsubstituted heteroaryl;
A is —N(R⁵)₂, wherein each instance of R⁵ is independently hydrogen; acyl; silyl; sulfonyl; an amino protecting group; substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; substituted or unsubstituted carbocyclyl; substituted or unsubstituted heterocyclyl; substituted or unsubstituted aryl; substituted or unsubstituted heteroaryl; or a group of the formula (iii′): 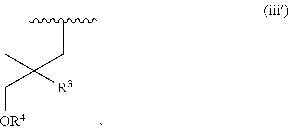
or two R⁵ groups are joined to form a substituted or unsubstituted heterocyclyl; and
Z is hydrogen; acyl; silyl; sulfonyl; an amino protecting group; substituted or unsubstituted alkyl; substituted or unsubstituted alkenyl; substituted or unsubstituted alkynyl; substituted or unsubstituted heteroalkyl; substituted or unsubstituted heteroalkenyl; substituted or unsubstituted heteroalkynyl; substituted or unsubstituted carbocyclyl; substituted or unsubstituted heterocyclyl; substituted or unsubstituted aryl; substituted or unsubstituted heteroaryl, or a group of the formula (iii′): 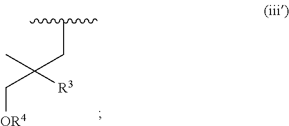
or Z and the nitrogen atom to which it is attached form a substituted or unsubstituted heterocyclyl group; and
a lipid-polyethylene glycol (PEG) conjugate of Formula (II): 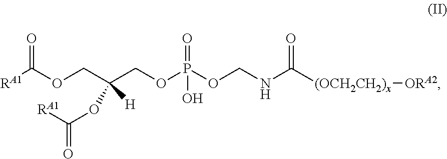
or a pharmaceutically acceptable salt thereof,
wherein:
each instance of R^{A1} is independently unsubstituted C₆-C₂₀ alkyl;
R^{A2} is substituted or unsubstituted alkyl; and
x is an integer between 15 to 135, inclusive.

2. (canceled)

3. The composition of claim 1, wherein at least one instance of L¹ is of the formula (iii-a)

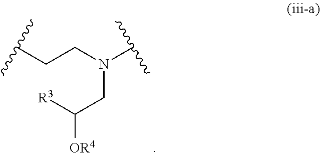

4. The composition of claim 1, wherein at least one instance of R³ is C₆-C₁₆ substituted or unsubstituted alkyl.

5-7. (canceled)

8. The composition of claim 1, wherein the conjugated lipomer of Formula (I) is of the formula: 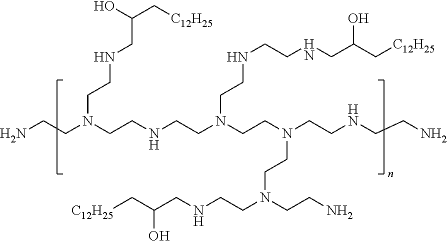
or a pharmaceutically acceptable salt thereof.

9. (canceled)

10. The composition claim 1, wherein the PEG of the lipid-PEG conjugate of Formula (II) has a molecular weight of approximately 600 g/mol to 750 g/mol, approximately 900 g/mol to 1100 g/mol, approximately 1500 g/mol to 2450 g/mol, approximately 2500 g/mol to 3000 g/mol, or approximately 4500 g/mol to 5500 g/mol.

11-13. (canceled)

14. The composition of claim 1, wherein R^{A1} is unsubstituted C₁₃ alkyl, unsubstituted C₁₅ alkyl, or unsubstituted C₁₇ alkyl.

15. The composition of claim 1, wherein R^{A2} is substituted or unsubstituted C₁-C₆ alkyl.

16. (canceled)

17. The composition of claim 1, wherein the lipid-PEG conjugate of Formula (II) is of the formula: 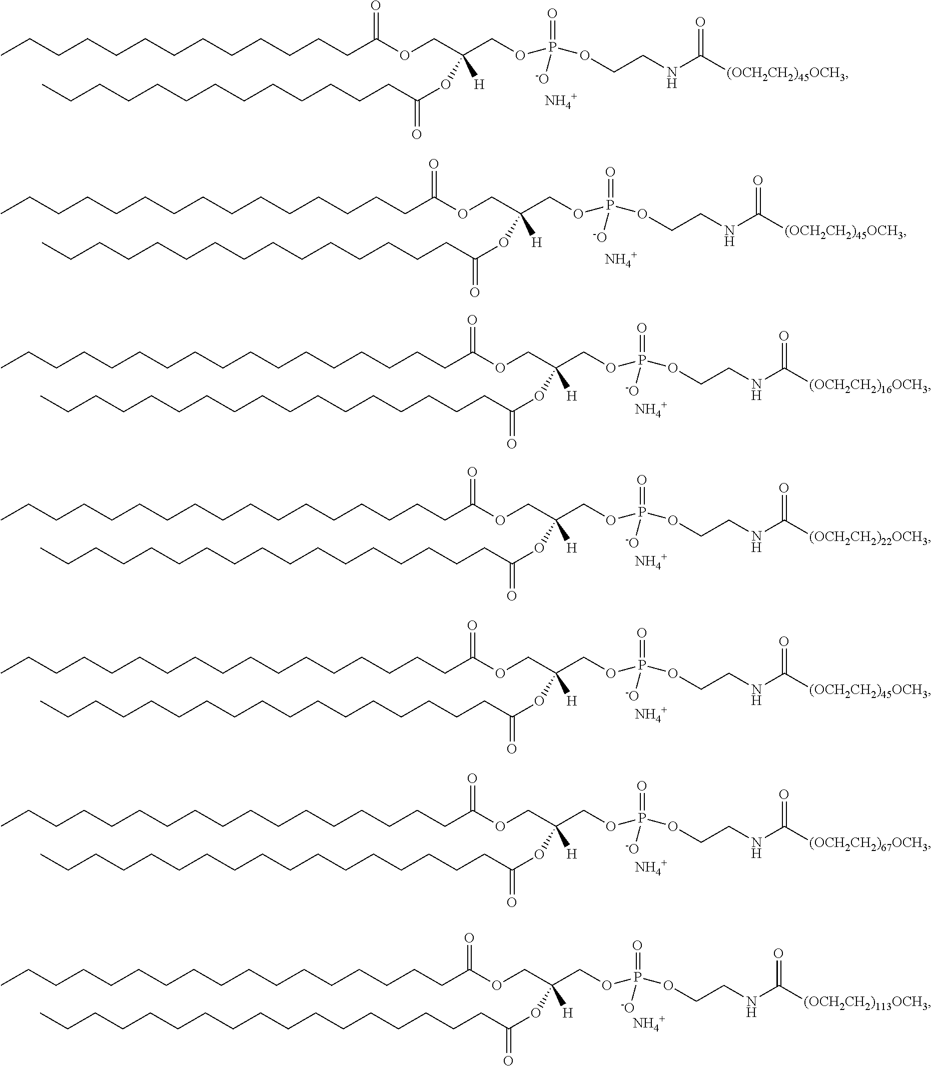
or a pharmaceutically acceptable salt thereof.

18-21. (canceled)

22. The composition of claim 1, further comprising an agent.

23. The composition of claim 22, wherein the agent is an organic molecule, inorganic molecule, nucleic acid, protein, peptide, polynucleotide, targeting agent, isotopically labeled chemical compound, vaccine, or an immunological agent.

24-25. (canceled)

26. The composition of claim 23, wherein the agent is a polynucleotide and the polynucleotide is RNA.

27. (canceled)

28. The composition of claim 26, wherein the RNA is siRNA.

29. The composition of claim 28, wherein the siRNA targets a protein of a hematopoietic stem and progenitor cells (HSPC).

30. The composition of claim 28, wherein the siRNA targets a protein of a bone marrow endothelial cells (BMEC).

31. The composition of claim 28, wherein the siRNA targets Sdf-1, Mcp-1, or Tie2.

32-35. (canceled)

36. A method for delivering an agent to a cell, comprising contacting the cell with a composition according to claim 23.

37-38. (canceled)

39. A method for delivering an agent to a subject, comprising administering to the subject a composition according to claim 23.

40-46. (canceled)

47. A method of treating a disease in a subject in need thereof, the method comprising administering to the subject a therapeutically effective amount of a composition according to claim 23.

48. (canceled)

49. The method of claim 47, wherein the disease is selected from the group consisting of cardiovascular disease, lung disease, proliferative disease, inflammatory disorders, and immunological disorders.

50-51. (canceled)

52. The method of claim 47, wherein the disease is a bone marrow disease or a hematological disorder.

53. (canceled)


SEQUENCE LISTING (26 sequences)

SEQ ID NO: 1 - DNA, 24 bp, Artificial Sequence
  Synthetic polynucleotide
  misc_feature (6)..(6): modified by 2′-OCH3
  misc_feature (8)..(8): modified by 2′-OCH3
  misc_feature (11)..(11): modified by 2′-OCH3
  misc_feature (14)..(16): modified by 2′-OCH3
  misc_feature (18)..(18): modified by 2′-OCH3
  misc_feature (20)..(20): modified by 2′-OCH3
  misc_feature (22)..(23): modified by 2′-OCH3
gaagaugcagugauuuacadtsdt

SEQ ID NO: 2 - DNA, 24 bp, Artificial Sequence
  Synthetic polynucleotide
  misc_feature (3)..(3): modified by 2′-OCH3
  misc_feature (8)..(8): modified by 2′-OCH3
  misc_feature (13)..(13): modified by 2′-OCH3
  misc_feature (20)..(20): modified by 2′-OCH3
  misc_feature (22)..(23): modified by 2′-OCH3
uguaaaucacugcaucuucdtsdt

SEQ ID NO: 3 - DNA, 24 bp, Artificial Sequence
  Synthetic polynucleotide
  misc_feature (1)..(3): modified by 2′-OCH3
  misc_feature (8)..(8): modified by 2′-OCH3
  misc_feature (10)..(12): modified by 2′-OCH3
  misc_feature (14)..(14): modified by 2′-OCH3
  misc_feature (16)..(16): modified by 2′-OCH3
  misc_feature (18)..(20): modified by 2′-OCH3
  misc_feature (22)..(23): modified by 2′-OCH3
gcuggagcauccacguguudtsdt

SEQ ID NO: 4 - DNA, 24 bp, Artificial Sequence
  Synthetic polynucleotide
  misc_feature (3)..(3): modified by 2′-OCH3
  misc_feature (16)..(16): modified by 2′-OCH3
  misc_feature (20)..(20): modified by 2′-OCH3
  misc_feature (22)..(23): modified by 2′-OCH3
aacacguggaugcuccagcdtsdt

SEQ ID NO: 5 - DNA, 24 bp, Artificial Sequence
  Synthetic polynucleotide
  misc_feature (1)..(2): modified by 2′-OCH3
  misc_feature (4)..(4): modified by 2′-OCH3
  misc_feature (6)..(6): modified by 2′-OCH3
  misc_feature (8)..(9): modified by 2′-OCH3
  misc_feature (12)..(14): modified by 2′-OCH3
  misc_feature (17)..(18): modified by 2′-OCH3
  misc_feature (20)..(20): modified by 2′-OCH3
  misc_feature (22)..(23): modified by 2′-OCH3
ccacguguuggcucagccadtsdt

SEQ ID NO: 6 - DNA, 24 bp, Artificial Sequence
  Synthetic polynucleotide
  misc_feature (10)..(10): modified by 2′-OCH3
  misc_feature (13)..(13): modified by 2′-OCH3
  misc_feature (20)..(20): modified by 2′-OCH3
  misc_feature (22)..(23): modified by 2′-OCH3
uggcugagccaacacguggdtsdt

SEQ ID NO: 7 - DNA, 24 bp, Artificial Sequence
  Synthetic polynucleotide
  misc_feature (1)..(3): modified by 2′-OCH3
  misc_feature (12)..(12): modified by 2′-OCH3
  misc_feature (14)..(14): modified by 2′-OCH3
  misc_feature (17)..(20): modified by 2′-OCH3
  misc_feature (22)..(23): modified by 2′-OCH3
cccaaagaagcuguaguuudtsdt

SEQ ID NO: 8 - DNA, 24 bp, Artificial Sequence
  Synthetic polynucleotide
  misc_feature (5)..(5): modified by 2′-OCH3
  misc_feature (7)..(7): modified by 2′-OCH3
  misc_feature (20)..(20): modified by 2′-OCH3
  misc_feature (22)..(23): modified by 2′-OCH3
aaacuacagcuucuuugggdtsdt

SEQ ID NO: 9 - DNA, 24 bp, Artificial Sequence
  Synthetic polynucleotide
  misc_feature (1)..(2): modified by 2′-OCH3
  misc_feature (5)..(6): modified by 2′-OCH3
  misc_feature (8)..(8): modified by 2′-OCH3
  misc_feature (11)..(15): modified by 2′-OCH3
  misc_feature (17)..(18): modified by 2′-OCH3
  misc_feature (20)..(20): modified by 2′-OCH3
  misc_feature (22)..(23): modified by 2′-OCH3
gaagcuguaguuuuugucadtsdt

SEQ ID NO: 10 - DNA, 24 bp, Artificial Sequence
  Synthetic polynucleotide
  misc_feature (4)..(4): modified by 2′-OCH3
  misc_feature (11)..(11): modified by 2′-OCH3
  misc_feature (13)..(13): modified by 2′-OCH3
  misc_feature (20)..(20): modified by 2′-OCH3
  misc_feature (22)..(23): modified by 2′-OCH3
ugacaaaaacuacagcuucdtsdt

SEQ ID NO: 11 - DNA, 24 bp, Artificial Sequence
  Synthetic polynucleotide
  misc_feature (1)..(2): modified by 2′-OCH3
  misc_feature (4)..(5): modified by 2′-OCH3
  misc_feature (8)..(8): modified by 2′-OCH3
  misc_feature (10)..(11): modified by 2′-OCH3
  misc_feature (14)..(15): modified by 2′-OCH3
  misc_feature (17)..(20): modified by 2′-OCH3
  misc_feature (22)..(23): modified by 2′-OCH3
gagccaacgucaagcaucudtsdt

SEQ ID NO: 12 - DNA, 24 bp, Artificial Sequence
  Synthetic polynucleotide
  misc_feature (20)..(20): modified by 2′-OCH3
  misc_feature (22)..(23): modified by 2′-OCH3
agaugcuugacguuggcucdtsdt

SEQ ID NO: 13 - DNA, 24 bp, Artificial Sequence
  Synthetic polynucleotide
  misc_feature (1)..(2): modified by 2′-OCH3
  misc_feature (5)..(5): modified by 2′-OCH3
  misc_feature (7)..(8): modified by 2′-OCH3
  misc_feature (12)..(12): modified by 2′-OCH3
  misc_feature (14)..(16): modified by 2′-OCH3
  misc_feature (20)..(20): modified by 2′-OCH3
  misc_feature (22)..(23): modified by 2′-OCH3
ccaacgucaagcaucugaadtsdt

SEQ ID NO: 14 - DNA, 24 bp, Artificial Sequence
  Synthetic polynucleotide
  misc_feature (3)..(3): modified by 2′-OCH3
  misc_feature (20)..(20): modified by 2′-OCH3
  misc_feature (22)..(23): modified by 2′-OCH3
uucagaugcuugacguuggdtsdt

SEQ ID NO: 15 - DNA, 24 bp, Artificial Sequence
  Synthetic polynucleotide
  misc_feature (1)..(3): modified by 2′-OCH3
  misc_feature (9)..(14): modified by 2′-OCH3
  misc_feature (16)..(16): modified by 2′-OCH3
  misc_feature (18)..(20): modified by 2′-OCH3
  misc_feature (22)..(23): modified by 2′-OCH3
ucugaaaauccucaacacudtsdt

SEQ ID NO: 16 - DNA, 24 bp, Artificial Sequence
  Synthetic polynucleotide
  misc_feature (16)..(16): modified by 2′-OCH3
  misc_feature (20)..(20): modified by 2′-OCH3
  misc_feature (22)..(23): modified by 2′-OCH3
aguguugaggauuuucagadtsdt

SEQ ID NO: 17 - DNA, 24 bp, Artificial Sequence
  Synthetic polynucleotide
  misc_feature (1)..(6): modified by 2′-OCH3
  misc_feature (9)..(9): modified by 2′-OCH3
  misc_feature (11)..(14): modified by 2′-OCH3
  misc_feature (18)..(20): modified by 2′-OCH3
  misc_feature (22)..(23): modified by 2′-OCH3
auccucaacacuccaaacudtsdt

SEQ ID NO: 18 - DNA, 24 bp, Artificial Sequence
  Synthetic polynucleotide
  misc_feature (20)..(20): modified by 2′-OCH3
  misc_feature (22)..(23): modified by 2′-OCH3
aguuuggaguguugaggaudtsdt

SEQ ID NO: 19 - DNA, 24 bp, Artificial Sequence
  Synthetic polynucleotide
  misc_feature (1)..(4): modified by 2′-OCH3
  misc_feature (8)..(9): modified by 2′-OCH3
  misc_feature (11)..(11): modified by 2′-OCH3
  misc_feature (13)..(18): modified by 2′-OCH3
  misc_feature (20)..(20): modified by 2′-OCH3
  misc_feature (22)..(23): modified by 2′-OCH3
cuccaaacugugcccuucadtsdt

SEQ ID NO: 20 - DNA, 24 bp, Artificial Sequence
  Synthetic polynucleotide
  misc_feature (8)..(8): modified by 2′-OCH3
  misc_feature (10)..(10): modified by 2′-OCH3
  misc_feature (20)..(20): modified by 2′-OCH3
  misc_feature (22)..(23): modified by 2′-OCH3
ugaagggcacaguuuggagdtsdt

SEQ ID NO: 21 - DNA, 24 bp, Artificial Sequence
  Synthetic polynucleotide
  misc_feature (1)..(3): modified by 2′-OCH3
  misc_feature (5)..(6): modified by 2′-OCH3
  misc_feature (8)..(8): modified by 2′-OCH3
  misc_feature (10)..(10): modified by 2′-OCH3
  misc_feature (13)..(14): modified by 2′-OCH3
  misc_feature (20)..(20): modified by 2′-OCH3
  misc_feature (22)..(23): modified by 2′-OCH3
auuguugcacggcugaagadtsdt

SEQ ID NO: 22 - DNA, 24 bp, Artificial Sequence
  Synthetic polynucleotide
  misc_feature (5)..(5): modified by 2′-OCH3
  misc_feature (13)..(13): modified by 2′-OCH3
  misc_feature (16)..(16): modified by 2′-OCH3
  misc_feature (20)..(20): modified by 2′-OCH3
  misc_feature (22)..(23): modified by 2′-OCH3
ucuucagccgugcaacaaudtsdt

SEQ ID NO: 23 - DNA, 24 bp, Artificial Sequence
  Synthetic polynucleotide
  misc_feature (1)..(2): modified by 2′-OCH3
  misc_feature (4)..(4): modified by 2′-OCH3
  misc_feature (6)..(6): modified by 2′-OCH3
  misc_feature (9)..(10): modified by 2′-OCH3
  misc_feature (14)..(14): modified by 2′-OCH3
  misc_feature (17)..(17): modified by 2′-OCH3
  misc_feature (20)..(20): modified by 2′-OCH3
  misc_feature (22)..(23): modified by 2′-OCH3
uugcacggcugaagaacaadtsdt

SEQ ID NO: 24 - DNA, 24 bp, Artificial Sequence
  Synthetic polynucleotide
  misc_feature (9)..(9): modified by 2′-OCH3
  misc_feature (17)..(17): modified by 2′-OCH3
  misc_feature (20)..(20): modified by 2′-OCH3
  misc_feature (22)..(23): modified by 2′-OCH3
uuguucuucagccgugcaadtsdt

SEQ ID NO: 25 - DNA, 24 bp, Artificial Sequence
  Synthetic polynucleotide
  misc_feature (1)..(2): modified by 2′-OCH3
  misc_feature (4)..(4): modified by 2′-OCH3
  misc_feature (6)..(6): modified by 2′-OCH3
  misc_feature (8)..(8): modified by 2′-OCH3
  misc_feature (10)..(11): modified by 2′-OCH3
  misc_feature (14)..(16): modified by 2′-OCH3
  misc_feature (20)..(20): modified by 2′-OCH3
  misc_feature (22)..(23): modified by 2′-OCH3
aagugugcauugacccgaadtsdt

SEQ ID NO: 26 - DNA, 24 bp, Artificial Sequence
  Synthetic polynucleotide
  misc_feature (8)..(8): modified by 2′-OCH3
  misc_feature (13)..(13): modified by 2′-OCH3
  misc_feature (15)..(15): modified by 2′-OCH3
  misc_feature (20)..(20): modified by 2′-OCH3
  misc_feature (22)..(23): modified by 2′-OCH3
uucgggucaaugcacacuudtsdt